(19) United States
(12) Patent Application Publication
(10) Pub. No.: US 2026/0146289 A1
(43) Pub. Date: May 28, 2026

(54) ASSESSING AND TREATING MELANOMA

(71) Applicant: Mayo Foundation for Medical Education and Research, Rochester, MN (US)

(72) Inventor: Alexander Meves, Rochester, MN (US)

(21) Appl. No.: 18/717,302

(22) PCT Filed: Dec. 1, 2022
(86) PCT No.: PCT/US2022/051518

§ 371 Date: Jun. 6, 2024

Related U.S. Application Data
(60) Provisional application No. 63/287,218, filed on Dec. 8, 2021.

(51) Int. Cl. C12Q 1/6886

(52) U.S. Cl. CPC C12Q 1/6886; C12Q 2600/118; C12Q 2600/158

(57) ABSTRACT

Methods and materials for assessing and/or treating mammals, e.g., humans, having melanoma, e.g., primary cutaneous melanoma. For example, methods and materials that can be used to determine whether or not a melanoma, e.g., a PCM, is likely to metastasize, e.g., likely to metastasize to one or more lymph nodes, are provided. For example, methods and materials for treating a mammal, e.g., a human, having melanoma, e.g., PCM, where the treatment is selected based, at least in part, on whether or not the melanoma is likely to metastasize are provided.

### CROSS-REFERENCE TO RELATED APPLICATIONS

This application is a national phase entry under 35 U.S.C. § 371 of International Patent Application PCT/US2022/051518, filed Dec. 1, 2022, designating the United States of America and published as International Patent Publication WO 2023/107329 A1 on Jun. 15, 2023, which claims the benefit under Article 8 of the Patent Cooperation Treaty of U.S. Provisional Application Ser. No. 63/287,218, filed Dec. 8, 2021. The disclosure of the prior application is considered part of (and is incorporated by reference in) the disclosure of this application.

### TECHNICAL FIELD

This document relates to methods and materials for assessing and/or treating mammals (e.g., humans) having melanoma (e.g., primary cutaneous melanoma (PCM)). For example, methods and materials provided herein can be used to determine whether or not a melanoma (e.g., a PCM) is likely to metastasize (e.g., likely to metastasize to one or more lymph nodes). This document also provides methods and materials for treating a mammal (e.g., a human) having melanoma (e.g., PCM) where the treatment is selected based, at least in part, on whether or not the melanoma is likely to metastasize.

### BACKGROUND INFORMATION

PCM accounts for only about 1-2% of all skin cancer diagnosed, but because of its extraordinary ability to metastasize PCM accounts for approximately 75% of skin cancer deaths. At present, nodal metastasis is determined by pathologic examination of sentinel lymph nodes (SLNs) obtained by SLN biopsy (SLNb). However, according to current national and international guidelines (Swetter et al., J. Natl. Compr. Canc. Netw. 19(4):364-376 (2021)), SLNb is not recommended if the risk of nodal metastasis is <5%, for example, in melanoma with a Breslow thickness of <0.8 mm and no adverse features. Nodal metastasis is found in <20% of patients who undergo a SLNb; however, all patients who undergo SLNb face a >10% risk of complications, as well as an up to 5% risk of hospital readmission within 30 days because of postsurgical complications (Gershenwald et al., CA Cancer J. Clin., 67:472-92 (2017)).

### SUMMARY

This document provides methods and materials for assessing and/or treating melanoma (e.g., PCM). In some cases, this document provides methods and materials for determining whether or not a melanoma (e.g., a PCM) is likely to metastasize (e.g., likely to metastasize to one or more lymph nodes). For example, a sample (e.g., a tissue sample containing one or more melanoma cells) obtained from a mammal having melanoma (e.g., PCM) can be assessed to determine if the melanoma is likely to metastasize based, at least in part, on the molecular signature of the melanoma. For example, a sample (e.g., a tissue sample containing one or more melanoma cells) obtained from a mammal having melanoma (e.g., PCM) can be assessed for (a) the presence or absence of altered levels (e.g., increased levels or decreased levels) of 11 or more (e.g., eleven, twelve, thirteen, or fourteen) polypeptides (or mRNA encoding such polypeptides), and/or (b) the presence or absence of one or more (e.g., one, two, or three) mutations in one or more polypeptides (e.g., a BRAF polypeptide and a NRAS polypeptide) to determine whether or not that melanoma is likely to metastasize. This document also provides methods and materials for treating a mammal (e.g., a human) having melanoma (e.g., PCM) where the treatment is selected based, at least in part, on whether or not the melanoma is likely to metastasize. For example, a mammal (e.g., a human) having melanoma (e.g., PCM) can be administered one or more cancer treatments that are selected based, at least in part, on the molecular signature of the melanoma.

As demonstrated herein, the presence of an increased level of a transmembrane Protein 163 (TMEM163) polypeptide (or mRNA encoding a TMEM163 polypeptide), an increased level of a hairy/enhancer-of-split related with YRPW motif protein 1 (HEY1) polypeptide (or mRNA encoding a HEY1 polypeptide), an increased level of a EYA transcriptional coactivator and phosphatase 4 (EYA4) polypeptide (or mRNA encoding an EYA polypeptide), an increased level of a transmembrane protein 98 (TMEM98) polypeptide (or mRNA encoding a TMEM98 polypeptide), an increased level of a retinaldehyde binding protein 1 (RLBP1) polypeptide (or mRNA encoding a RLBP1 polypeptide), an increased level of an elongation of very-long-chain fatty acids-like 2 (ELOVL2) polypeptide (or mRNA encoding an ELOVL2 polypeptide), an increased level of a serpin family E member 2 (SERPINE2) polypeptide (or mRNA encoding a SERPINE2 polypeptide), an increased level of an integrin subunit beta 3 (ITGB3) polypeptide (or mRNA encoding an ITGB3 polypeptide), an increased level of an insulin like growth factor 1 receptor (IGF1R) polypeptide (or mRNA encoding an IGF1R polypeptide), a decreased level of a cell surface glycoprotein CD200 receptor 1 (CD200R1) polypeptide (or mRNA encoding a CD200R1 polypeptide), a decreased level of a signal regulatory protein gamma (SIRPG) polypeptide (or mRNA encoding a SIRPG polypeptide), a decreased level of a betacellulin (BTC) polypeptide (or mRNA encoding a BTC polypeptide), a decreased level of an olfactory receptor family 52 subfamily N member 4 (OR52N4) polypeptide (or mRNA encoding an OR52N4 polypeptide), a decreased level of a ETS variant transcription factor 7 (ETV7) polypeptide (or mRNA encoding an ETV7 polypeptide), a decreased level of a thymocyte selection-associated high mobility group box (TOX) polypeptide (or mRNA encoding a TOX polypeptide), a decreased level of an arylsulfatase G (ARSG) polypeptide (or mRNA encoding an ARSG polypeptide), a decreased level of an integrin subunit beta 7 (ITGB7) polypeptide (or mRNA encoding an ITGB7 polypeptide), a decreased level of a cyclin dependent kinase inhibitor 2A (CDKN2A) polypeptide (or mRNA encoding a CDKN2A polypeptide), and a decreased level of a dermcidin (DCD) polypeptide (or mRNA encoding a DCD polypeptide) in melanoma cells of a mammal having melanoma (e.g., PCM) can be used to identify that melanoma as being likely to metastasize to one or more lymph nodes. Also as demonstrated herein, the presence of one or more mutations in a BRAF polypeptide and the presence of one or more mutations in a NRAS polypeptide in melanoma cells of a mammal having melanoma (e.g., PCM) can be used to identify that melanoma as being likely to metastasize to one or more lymph nodes.

Having the ability to identify whether a melanoma (e.g., PCM) is likely to metastasize (e.g., likely to metastasize to one or more lymph nodes) as described herein (e.g., based, at least in part, on the molecular signature of the melanoma) provides a unique and unrealized opportunity to provide an individualized approach for selecting cancer therapies based on the risk of the patient, thus providing cost-effective care with better outcomes. For example, a mammal having a high-risk melanoma (e.g., a melanoma that is identified as being likely to metastasize as described herein) can be selected for adjuvant therapies, increased screening, and/or participation in clinical trials. For example, a mammal having a low-risk melanoma (e.g., a melanoma that is identified as not being likely to metastasize as described herein) can forgo unnecessary SLNb and be spared from the risks and complications associated with SLNb.

In general, one aspect of this document features methods for assessing a mammal having a PCM. The methods can include, or consist essentially of, (a) determining if a sample from a mammal having a PCM contains the presence or absence of (1) an increased level of a TMEM163 polypeptide or mRNA encoding the TMEM163 polypeptide, (2) an increased level of a HEY1 polypeptide or mRNA encoding the HEY1 polypeptide, (3) an increased level of an EYA4 polypeptide or mRNA encoding the EYA4 polypeptide, (4) an increased level of a TMEM98 polypeptide or mRNA encoding the TMEM98 polypeptide, (5) an increased level of a RLBP1 polypeptide or mRNA encoding the RLBP1 polypeptide, (6) an increased level of an ELOVL2 polypeptide or mRNA encoding the ELOVL2 polypeptide, (7) an increased level of a SERPINE2 polypeptide or mRNA encoding the SERPINE2 polypeptide, (8) an increased level of an ITGB3 polypeptide or mRNA encoding the ITGB3 polypeptide, (9) an increased level of an IGF1R polypeptide or mRNA encoding the IGF1R polypeptide, (10) a decreased level of a CD200R1 polypeptide or mRNA encoding the CD200R1 polypeptide, (11) a decreased level of a SIRPG polypeptide or mRNA encoding the SIRPG polypeptide, (12) a decreased level of a BTC polypeptide or mRNA encoding the BTC polypeptide, (13) a decreased level of an OR52N4 polypeptide or mRNA encoding the OR52N4 polypeptide, (14) a decreased level of an ETV7 polypeptide or mRNA encoding the ETV7 polypeptide, (15) a decreased level of a TOX polypeptide or mRNA encoding the TOX polypeptide, (16) a decreased level of an ARSG polypeptide or mRNA encoding the ARSG polypeptide, (17) a decreased level of an ITGB7 polypeptide or mRNA encoding the ITGB7 polypeptide, (18) a decreased level of a CDKN2A polypeptide or mRNA encoding the CDKN2A polypeptide, or (19) a decreased level of a DCD polypeptide or mRNA encoding the DCD polypeptide; (b) classifying the PCM as being likely to metastasize if the presence of at least 11 of (1)-(19) are determined; and (c) classifying the PCM as not being likely to metastasize if the absence of each of (1)-(19) is determined. The mammal can be a human. The presence or absence of the increased level of the TMEM163 polypeptide or mRNA encoding the TMEM163 polypeptide can be determined. The presence or absence of the increased level of the HEY1 polypeptide or mRNA encoding the HEY1 polypeptide can be determined. The presence or absence of the increased level of the EYA4 polypeptide or mRNA encoding the EYA4 polypeptide can be determined. The presence or absence of the increased level of the TMEM98 polypeptide or mRNA encoding the TMEM98 polypeptide can be determined. The presence or absence of the increased level of the RLBP1 polypeptide or mRNA encoding the RLBP1 polypeptide can be determined. The presence or absence of the increased level of the ELOVL2 polypeptide or mRNA encoding the ELOVL2 polypeptide can be determined. The presence or absence of the increased level of the SERPINE2 polypeptide or mRNA encoding the SERPINE2 polypeptide can be determined. The presence or absence of the increased level of the ITGB3 polypeptide or mRNA encoding the ITGB3 polypeptide can be determined. The presence or absence of the increased level of the IGF1R polypeptide or mRNA encoding the IGF1R polypeptide can be determined. The presence or absence of the decreased level of the CD200R1 polypeptide or mRNA encoding the CD200R1 polypeptide can be determined. The presence or absence of the decreased level of the SIRPG polypeptide or mRNA encoding the SIRPG polypeptide can be determined. The presence or absence of the decreased level of the BTC polypeptide or mRNA encoding the BTC polypeptide can be determined. The presence or absence of the decreased level of the OR52N4 polypeptide or mRNA encoding the OR52N4 polypeptide can be determined. The presence or absence of the decreased level of the ETV7 polypeptide or mRNA encoding the ETV7 polypeptide can be determined. The presence or absence of the decreased level of the TOX polypeptide or mRNA encoding the TOX polypeptide can be determined. The presence or absence of the decreased level of the ARSG polypeptide or mRNA encoding the ARSG polypeptide can be determined. The presence or absence of the decreased level of the ITGB7 polypeptide or mRNA encoding the ITGB7 polypeptide can be determined. The presence or absence of the decreased level of the CDKN2A polypeptide or mRNA encoding the CDKN2A polypeptide can be determined. The presence or absence of the decreased level of the DCD polypeptide or mRNA encoding the DCD polypeptide can be determined. The method also can include determining the presence or absence of a mutation in a BRAF polypeptide. The mutation in the BRAF polypeptide can be an amino acid substitution at residue 600 of the BRAF polypeptide. The mutation in the BRAF polypeptide can be a BRAFV600E substitution, a BRAFV600K substitution, a BRAFV600R substitution, or a BRAFV600D substitution. The method also can include determining the presence or absence of a mutation in a NRAS polypeptide. The mutation in the NRAS polypeptide can be an amino acid substitution at residue 61 of the NRAS polypeptide. The mutation in the NRAS polypeptide can be a NRASQ61K substitution, a NRASQ61R substitution, a NRASQ61L substitution, a NRASQ61V substitution, or a NRASQ61H substitution. The method also can include determining a Breslow depth of the PCM. In some cases, the method can include determining the presence of the increased level of the TMEM163 polypeptide or mRNA encoding the TMEM163 polypeptide, the increased level of the HEY1 polypeptide or mRNA encoding the HEY1 polypeptide, the increased level of the EYA4 polypeptide or mRNA encoding the EYA4 polypeptide, the increased level of the TMEM98 polypeptide or mRNA encoding the TMEM98 polypeptide, the increased level of the RLBP1 polypeptide or mRNA encoding the RLBP1 polypeptide, the increased level of the ELOVL2 polypeptide or mRNA encoding the ELOVL2 polypeptide, the increased level of the SERPINE2 polypeptide or mRNA encoding the SERPINE2 polypeptide, the increased level of the ITGB3 polypeptide or mRNA encoding the ITGB3 polypeptide, the increased level of the IGF1R polypeptide or mRNA encoding the IGF1R polypeptide, the decreased level of the CD200R1 polypeptide or mRNA encoding the CD200R1 polypeptide, the decreased level of the SIRPG polypeptide or mRNA encoding the SIRPG polypeptide, the decreased level of the BTC polypeptide or mRNA encoding the BTC polypeptide, the decreased level of the OR52N4 polypeptide or mRNA encoding the OR52N4 polypeptide, the decreased level of the ETV7 polypeptide or mRNA encoding the ETV7 polypeptide, the decreased level of the TOX polypeptide or mRNA encoding the TOX polypeptide, the decreased level of the ARSG polypeptide or mRNA encoding the ARSG polypeptide, the decreased level of the ITGB7 polypeptide or mRNA encoding the ITGB7 polypeptide, the decreased level of the CDKN2A polypeptide or mRNA encoding the CDKN2A polypeptide, and the decreased level of a DCD polypeptide or mRNA encoding the DCD polypeptide; determining that the Breslow depth is from about 0.8 mm to about 10 mm; and can include classifying the PCM as being likely to metastasize. In some cases, the method can include determining the absence of the increased level of the TMEM163 polypeptide or mRNA encoding the TMEM163 polypeptide, the increased level of the HEY1 polypeptide or mRNA encoding the HEY1 polypeptide, the increased level of the EYA4 polypeptide or mRNA encoding the EYA4 polypeptide, the increased level of the TMEM98 polypeptide or mRNA encoding the TMEM98 polypeptide, the increased level of the RLBP1 polypeptide or mRNA encoding the RLBP1 polypeptide, the increased level of the ELOVL2 polypeptide or mRNA encoding the ELOVL2 polypeptide, the increased level of the SERPINE2 polypeptide or mRNA encoding the SERPINE2 polypeptide, the increased level of the ITGB3 polypeptide or mRNA encoding the ITGB3 polypeptide, the increased level of the IGF1R polypeptide or mRNA encoding the IGF1R polypeptide, the decreased level of the CD200R1 polypeptide or mRNA encoding the CD200R1 polypeptide, the decreased level of the SIRPG polypeptide or mRNA encoding the SIRPG polypeptide, the decreased level of the BTC polypeptide or mRNA encoding the BTC polypeptide, the decreased level of the OR52N4 polypeptide or mRNA encoding the OR52N4 polypeptide, the decreased level of the ETV7 polypeptide or mRNA encoding the ETV7 polypeptide, the decreased level of the TOX polypeptide or mRNA encoding the TOX polypeptide, the decreased level of the ARSG polypeptide or mRNA encoding the ARSG polypeptide, the decreased level of the ITGB7 polypeptide or mRNA encoding the ITGB7 polypeptide, the decreased level of the CDKN2A polypeptide or mRNA encoding the CDKN2A polypeptide, and the decreased level of the DCD polypeptide or mRNA encoding the DCD polypeptide; and determining that the Breslow depth is from about 0.1 mm to about 0.8 mm; and can include classifying the PCM as not being likely to metastasize. The sample can be a tissue sample comprising a melanoma cell.

In another aspect, this document features methods for selecting a treatment for a mammal having a PCM. The methods can include, or consist essentially of, (a) determining that a sample from a mammal having a PCM contains the presence of at least 11 of: (1) an increased level of a TMEM163 polypeptide or mRNA encoding the TMEM163 polypeptide, (2) an increased level of a HEY1 polypeptide or mRNA encoding the HEY1 polypeptide, (3) an increased level of an EYA4 polypeptide or mRNA encoding the EYA4 polypeptide, (4) an increased level of a TMEM98 polypeptide or mRNA encoding the TMEM98 polypeptide, (5) an increased level of a RLBP1 polypeptide or mRNA encoding the RLBP1 polypeptide, (6) an increased level of an ELOVL2 polypeptide or mRNA encoding the ELOVL2 polypeptide, (7) an increased level of a SERPINE2 polypeptide or mRNA encoding the SERPINE2 polypeptide, (8) an increased level of an ITGB3 polypeptide or mRNA encoding the ITGB3 polypeptide, (9) an increased level of an IGF1R polypeptide or mRNA encoding the IGF1R polypeptide, (10) a decreased level of a CD200R1 polypeptide or mRNA encoding the CD200R1 polypeptide, (11) a decreased level of a SIRPG polypeptide or mRNA encoding the SIRPG polypeptide, (12) a decreased level of a BTC polypeptide or mRNA encoding the BTC polypeptide, (13) a decreased level of an OR52N4 polypeptide or mRNA encoding the OR52N4 polypeptide, (14) a decreased level of an ETV7 polypeptide or mRNA encoding the ETV7 polypeptide, (15) a decreased level of a TOX polypeptide or mRNA encoding the TOX polypeptide, (16) a decreased level of an ARSG polypeptide or mRNA encoding the ARSG polypeptide, (17) a decreased level of an ITGB7 polypeptide or mRNA encoding the ITGB7 polypeptide, (18) a decreased level of a CDKN2A polypeptide or mRNA encoding the CDKN2A polypeptide, and (19) a decreased level of a DCD polypeptide or mRNA encoding the DCD polypeptide, where the sample is a tissue sample comprising a melanoma cell; and (b) selecting the mammal for surgery to remove the PCM and to receive an adjuvant therapy. The method also can include determining that the PCM has a Breslow depth of from about 0.8 mm to about 10 mm. The method further also can include determining that the sample contains the presence of a mutation in a BRAF polypeptide or a mutation in a NRAS polypeptide. The mutation in the BRAF polypeptide can be an amino acid substitution at residue 600 of the BRAF polypeptide. The mutation in the BRAF polypeptide can be a BRAFV600E substitution, a BRAFV600K substitution, a BRAFV600R substitution, or a BRAFV600D substitution. The mutation in the NRAS polypeptide can be an amino acid substitution at residue 61 of the NRAS polypeptide. The mutation in the NRAS polypeptide can be a NRASQ61K substitution, a NRASQ61R substitution, a NRASQ61L substitution, a NRASQ61V substitution, or a NRASQ61H substitution. The mammal can be a human. The adjuvant therapy can include radiation therapy. The adjuvant therapy can include administering an anti-cancer drug to the mammal. The anti-cancer can be nivolumab, ipilimumab, pembrolizumab, talimogene laherparepvec, vemurafenib, dabrafenib, encorafenib, trametinib, cobimetinib, binimetinib, atezolizumab, avelumab, or durvalumab.

In another aspect, this document features methods for selecting a treatment for a mammal having a PCM. The methods can include, or consist essentially of, (a) determining that a sample from a mammal having a PCM contains the absence of: (1) an increased level of a TMEM163 polypeptide or mRNA encoding the TMEM163 polypeptide, (2) an increased level of a HEY1 polypeptide or mRNA encoding the HEY1 polypeptide, (3) an increased level of an EYA4 polypeptide or mRNA encoding the EYA4 polypeptide, (4) an increased level of a TMEM98 polypeptide or mRNA encoding the TMEM98 polypeptide, (5) an increased level of a RLBP1 polypeptide or mRNA encoding the RLBP1 polypeptide, (6) an increased level of an ELOVL2 polypeptide or mRNA encoding the ELOVL2 polypeptide, (7) an increased level of a SERPINE2 polypeptide or mRNA encoding the SERPINE2 polypeptide, (8) an increased level of an ITGB3 polypeptide or mRNA encoding the ITGB3 polypeptide, (9) an increased level of an IGF1R polypeptide or mRNA encoding the IGF1R polypeptide, (10) a decreased level of a CD200R1 polypeptide or mRNA encoding the CD200R1 polypeptide, (11) a decreased level of a SIRPG polypeptide or mRNA encoding the SIRPG polypeptide, (12) a decreased level of a BTC polypeptide or mRNA encoding the BTC polypeptide, (13) a decreased level of an OR52N4 polypeptide or mRNA encoding the OR52N4 polypeptide, (14) a decreased level of an ETV7 polypeptide or mRNA encoding the ETV7 polypeptide, (15) a decreased level of a TOX polypeptide or mRNA encoding the TOX polypeptide, (16) a decreased level of an ARSG polypeptide or mRNA encoding the ARSG polypeptide, (17) a decreased level of an ITGB7 polypeptide or mRNA encoding the ITGB7 polypeptide, (18) a decreased level of a CDKN2A polypeptide or mRNA encoding the CDKN2A polypeptide, and (19) a decreased level of a DCD polypeptide or mRNA encoding the DCD polypeptide, where the sample is a tissue sample comprising a melanoma cell; and (b) selecting the mammal for surgery to remove the PCM in the absence of an adjuvant therapy. The method also can include determining that the PCM has a Breslow depth of from about 0.1 mm to about 0.8 mm. The method also can include determining that the sample contains the absence of a mutation in a BRAF polypeptide and a mutation in a NRAS polypeptide. The mutation in the BRAF polypeptide can be an amino acid substitution at residue 600 of the BRAF polypeptide. The mutation in the BRAF polypeptide can be a BRAFV600E substitution, a BRAFV600K substitution, a BRAFV600R substitution, or a BRAFV600D substitution. The mutation in the NRAS polypeptide can be an amino acid substitution at residue 61 of the NRAS polypeptide. The mutation in the NRAS polypeptide can be a NRASQ61K substitution, a NRASQ61R substitution, a NRASQ61L substitution, a NRASQ61V substitution, or a NRASQ61H substitution. The mammal can be a human. The adjuvant therapy can include radiation therapy. The adjuvant therapy can include administering an anti-cancer drug to the mammal. The anti-cancer can be nivolumab, ipilimumab, pembrolizumab, talimogene laherparepvec, vemurafenib, dabrafenib, encorafenib, trametinib, cobimetinib, binimetinib, atezolizumab, avelumab, or durvalumab.

In another aspect, this document features methods for treating a mammal having a PCM. The methods can include, or consist essentially of, (a) determining that a sample from a mammal having a PCM contains the presence of at least 11 of: (1) an increased level of a TMEM163 polypeptide or mRNA encoding the TMEM163 polypeptide, (2) an increased level of a HEY1 polypeptide or mRNA encoding the HEY1 polypeptide, (3) an increased level of an EYA4 polypeptide or mRNA encoding the EYA4 polypeptide, (4) an increased level of a TMEM98 polypeptide or mRNA encoding the TMEM98 polypeptide, (5) an increased level of a RLBP1 polypeptide or mRNA encoding the RLBP1 polypeptide, (6) an increased level of an ELOVL2 polypeptide or mRNA encoding the ELOVL2 polypeptide, (7) an increased level of a SERPINE2 polypeptide or mRNA encoding the SERPINE2 polypeptide, (8) an increased level of an ITGB3 polypeptide or mRNA encoding the ITGB3 polypeptide, (9) an increased level of an IGF1R polypeptide or mRNA encoding the IGF1R polypeptide, (10) a decreased level of a CD200R1 polypeptide or mRNA encoding the CD200R1 polypeptide, (11) a decreased level of a SIRPG polypeptide or mRNA encoding the SIRPG polypeptide, (12) a decreased level of a BTC polypeptide or mRNA encoding the BTC polypeptide, (13) a decreased level of an OR52N4 polypeptide or mRNA encoding the OR52N4 polypeptide, (14) a decreased level of an ETV7 polypeptide or mRNA encoding the ETV7 polypeptide, (15) a decreased level of a TOX polypeptide or mRNA encoding the TOX polypeptide, (16) a decreased level of an ARSG polypeptide or mRNA encoding the ARSG polypeptide, (17) a decreased level of an ITGB7 polypeptide or mRNA encoding the ITGB7 polypeptide, (18) a decreased level of a CDKN2A polypeptide or mRNA encoding the CDKN2A polypeptide, and (19) a decreased level of a DCD polypeptide or mRNA encoding the DCD polypeptide, where the sample is a tissue sample comprising a melanoma cell; (b) subjecting the mammal to a surgery to remove the PCM; and (c) administering an adjuvant therapy to the mammal. The method also can include determining that the PCM has a Breslow depth of from about 0.8 mm to about 10 mm. The method also can include determining that the sample contains the presence of a mutation in a BRAF polypeptide or a mutation in a NRAS polypeptide. The mutation in the BRAF polypeptide can be an amino acid substitution at residue 600 of the BRAF polypeptide. The mutation in the BRAF polypeptide can be a BRAFV600E substitution, a BRAFV600K substitution, a BRAFV600R substitution, or a BRAFV600D substitution. The mutation in the NRAS polypeptide can be an amino acid substitution at residue 61 of the NRAS polypeptide. The mutation in the NRAS polypeptide can be a NRASQ61K substitution, a NRASQ61R substitution, a NRASQ61L substitution, a NRASQ61V substitution, or a NRASQ61H substitution. The mammal can be a human. The adjuvant therapy can include radiation therapy. The adjuvant therapy can include administering an anti-cancer drug to the mammal. The anti-cancer can be nivolumab, ipilimumab, pembrolizumab, talimogene laherparepvec, vemurafenib, dabrafenib, encorafenib, trametinib, cobimetinib, binimetinib, atezolizumab, avelumab, or durvalumab.

In another aspect, this document features methods for treating a mammal having a PCM. The methods can include, or consist essentially of, subjecting a mammal having a PCM to a surgery to remove the PCM and administering an adjuvant therapy to the mammal, where the mammal is identified as having at least 11 of: (1) an increased level of a TMEM163 polypeptide or mRNA encoding the TMEM163 polypeptide, (2) an increased level of a HEY1 polypeptide or mRNA encoding the HEY1 polypeptide, (3) an increased level of an EYA4 polypeptide or mRNA encoding the EYA4 polypeptide, (4) an increased level of a TMEM98 polypeptide or mRNA encoding the TMEM98 polypeptide, (5) an increased level of a RLBP1 polypeptide or mRNA encoding the RLBP1 polypeptide, (6) an increased level of an ELOVL2 polypeptide or mRNA encoding the ELOVL2 polypeptide, (7) an increased level of a SERPINE2 polypeptide or mRNA encoding the SERPINE2 polypeptide, (8) an increased level of an ITGB3 polypeptide or mRNA encoding the ITGB3 polypeptide, (9) an increased level of an IGF1R polypeptide or mRNA encoding the IGF1R polypeptide, (10) a decreased level of a CD200R1 polypeptide or mRNA encoding the CD200R1 polypeptide, (11) a decreased level of a SIRPG polypeptide or mRNA encoding the CD200R1 polypeptide, (12) a decreased level of a BTC polypeptide or mRNA encoding the BTC polypeptide, (13) a decreased level of an OR52N4 polypeptide or mRNA encoding the OR52N4 polypeptide, (14) a decreased level of an ETV7 polypeptide or mRNA encoding the ETV7 polypeptide, (15) a decreased level of a TOX polypeptide or mRNA encoding the TOX polypeptide, (16) a decreased level of an ARSG polypeptide or mRNA encoding the ARSG polypeptide, (17) a decreased level of an ITGB7 polypeptide or mRNA encoding the ITGB7 polypeptide, (18) a decreased level of a CDKN2A polypeptide or mRNA encoding the CDKN2A polypeptide, and (19) a decreased level of a DCD polypeptide or mRNA encoding the DCD polypeptide, where the sample is a tissue sample comprising a melanoma cell. The method also can include determining that the PCM has a Breslow depth of from about 0.8 mm to about 10 mm. The method also can include determining that the sample contains the presence of a mutation in a BRAF polypeptide or a mutation in a NRAS polypeptide. The mutation in the BRAF polypeptide can be an amino acid substitution at residue 600 of the BRAF polypeptide. The mutation in the BRAF polypeptide can be a BRAFV600E substitution, a BRAFV600K substitution, a BRAFV600R substitution, or a BRAFV600D substitution. The mutation in the NRAS polypeptide can be an amino acid substitution at residue 61 of the NRAS polypeptide. The mutation in the NRAS polypeptide can be a NRASQ61K substitution, a NRASQ61R substitution, a NRASQ61L substitution, a NRASQ61V substitution, or a NRASQ61H substitution. The mammal can be a human. The adjuvant therapy can include radiation therapy. The adjuvant therapy can include administering an anti-cancer drug to the mammal. The anti-cancer can be nivolumab, ipilimumab, pembrolizumab, talimogene laherparepvec, vemurafenib, dabrafenib, encorafenib, trametinib, cobimetinib, binimetinib, atezolizumab, avelumab, or durvalumab.

In another aspect, this document features methods for treating a mammal having a PCM. The methods can include, or consist essentially of, (a) determining if a sample from a mammal having a PCM contains the absence of: (1) an increased level of a TMEM163 polypeptide or mRNA encoding the TMEM163 polypeptide, (2) an increased level of a HEY1 polypeptide or mRNA encoding the HEY1 polypeptide, (3) an increased level of an EYA4 polypeptide or mRNA encoding the HEY1 polypeptide, (4) an increased level of a TMEM98 polypeptide or mRNA encoding the TMEM98 polypeptide, (5) an increased level of a RLBP1 polypeptide or mRNA encoding the RLBP1 polypeptide, (6) an increased level of an ELOVL2 polypeptide or mRNA encoding the ELOVL2 polypeptide, (7) an increased level of a SERPINE2 polypeptide or mRNA encoding the SERPINE2 polypeptide, (8) an increased level of an ITGB3 polypeptide or mRNA encoding the ITGB3 polypeptide, (9) an increased level of an IGF1R polypeptide or mRNA encoding the IGF1R polypeptide, (10) a decreased level of a CD200R1 polypeptide or mRNA encoding the CD200R1 polypeptide, (11) a decreased level of a SIRPG polypeptide or mRNA encoding the SIRPG polypeptide, (12) a decreased level of a BTC polypeptide or mRNA encoding the BTC polypeptide, (13) a decreased level of an OR52N4 polypeptide or mRNA encoding the OR52N4 polypeptide, (14) a decreased level of an ETV7 polypeptide or mRNA encoding the ETV7 polypeptide, (15) a decreased level of a TOX polypeptide or mRNA encoding the TOX polypeptide, (16) a decreased level of an ARSG polypeptide or mRNA encoding the ARSG polypeptide, (17) a decreased level of an ITGB7 polypeptide or mRNA encoding the ITGB7 polypeptide, (18) a decreased level of a CDKN2A polypeptide or mRNA encoding the CDKN2A polypeptide, and (19) a decreased level of a DCD polypeptide or mRNA encoding the DCD polypeptide, where the sample is a tissue sample comprising a melanoma cell; and (b) subjecting the mammal to a surgery to remove the PCM in the absence of an adjuvant therapy. The method also can include determining that the PCM has a Breslow depth of from about 0.1 mm to about 0.8 mm. The method also can include determining that the sample lacks a mutation in a BRAF polypeptide and a mutation in a NRAS polypeptide. The mutation in the BRAF polypeptide can be an amino acid substitution at residue 600 of the BRAF polypeptide. The mutation in the BRAF polypeptide can be a BRAFV600E substitution, a BRAFV600K substitution, a BRAFV600R substitution, or a BRAFV600D substitution. The mutation in the NRAS polypeptide can be an amino acid substitution at residue 61 of the NRAS polypeptide. The mutation in the NRAS polypeptide can be a NRASQ61K substitution, a NRASQ61R substitution, a NRASQ61L substitution, a NRASQ61V substitution, or a NRASQ61H substitution. The mammal can be a human. The adjuvant therapy can include radiation therapy. The adjuvant therapy can include administering an anti-cancer drug to the mammal. The anti-cancer drug can be nivolumab, ipilimumab, pembrolizumab, talimogene laherparepvec, vemurafenib, dabrafenib, encorafenib, trametinib, cobimetinib, binimetinib, atezolizumab, avelumab, or durvalumab.

In another aspect, this document features methods for treating a mammal having a PCM. The methods can include, or consist essentially of, subjecting a mammal having a PCM to a surgery to remove the PCM in the absence of an adjuvant therapy, where the mammal is identified as lacking: (1) an increased level of a TMEM163 polypeptide or mRNA encoding the TMEM163 polypeptide, (2) an increased level of a HEY1 polypeptide or mRNA encoding the HEY1 polypeptide, (3) an increased level of an EYA4 polypeptide or mRNA encoding the EYA4 polypeptide, (4) an increased level of a TMEM98 polypeptide or mRNA encoding the TMEM98 polypeptide, (5) an increased level of a RLBP1 polypeptide or mRNA encoding the RLBP1 polypeptide, (6) an increased level of an ELOVL2 polypeptide or mRNA encoding the ELOVL2 polypeptide, (7) an increased level of a SERPINE2 polypeptide or mRNA encoding the SERPINE2 polypeptide, (8) an increased level of an ITGB3 polypeptide or mRNA encoding the ITGB3 polypeptide, (9) an increased level of an IGF1R polypeptide or mRNA encoding the IGF1R polypeptide, (10) a decreased level of a CD200R1 polypeptide or mRNA encoding the CD200R1 polypeptide, (11) a decreased level of a SIRPG polypeptide or mRNA encoding the SIRPG polypeptide, (12) a decreased level of a BTC polypeptide or mRNA encoding the BTC polypeptide, (13) a decreased level of an OR52N4 polypeptide or mRNA encoding the OR52N4 polypeptide, (14) a decreased level of an ETV7 polypeptide or mRNA encoding the ETV7 polypeptide, (15) a decreased level of a TOX polypeptide or mRNA encoding the TOX polypeptide, (16) a decreased level of an ARSG polypeptide or mRNA encoding the ARSG polypeptide, (17) a decreased level of an ITGB7 polypeptide or mRNA encoding the ITGB7 polypeptide, (18) a decreased level of a CDKN2A polypeptide or mRNA encoding the CDKN2A polypeptide, and (19) a decreased level of a DCD polypeptide or mRNA encoding the DCD polypeptide, where the sample is a tissue sample comprising a melanoma cell. The method also can include determining that the PCM has a Breslow depth of from about 0.1 mm to about 0.8 mm. The method also can include determining that the sample lacks a mutation in a BRAF polypeptide and a mutation in a NRAS polypeptide. The mutation in the BRAF polypeptide can be an amino acid substitution at residue 600 of the BRAF polypeptide. The mutation in the BRAF polypeptide can be a BRAFV600E substitution, a BRAFV600K substitution, a BRAFV600R substitution, or a BRAFV600D substitution. The mutation in the NRAS polypeptide can be an amino acid substitution at residue 61 of the NRAS polypeptide. The mutation in the NRAS polypeptide can be a NRASQ61K substitution, a NRASQ61R substitution, a NRASQ61L substitution, a NRASQ61V substitution, or a NRASQ61H substitution. The mammal can be a human. The adjuvant therapy can include radiation therapy. The adjuvant therapy can include administering an anti-cancer drug to the mammal. The anti-cancer drug can be nivolumab, ipilimumab, pembrolizumab, talimogene laherparepvec, vemurafenib, dabrafenib, encorafenib, trametinib, cobimetinib, binimetinib, atezolizumab, avelumab, or durvalumab.

Unless otherwise defined, all technical and scientific terms used herein have the same meaning as commonly understood by one of ordinary skill in the art to which this disclosure pertains. Although methods and materials similar or equivalent to those described herein can be used to practice the disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the disclosure are set forth in the accompanying drawings and the description below. Other features, objects, and advantages of the disclosure will be apparent from the description and drawings, and from the claims.

### BRIEF DESCRIPTION OF THE DRAWINGS

FIG. 1 shows an exemplary method for step-by-step filtering of genes to identify markers for predicting melanoma metastasis.

FIG. 2 contains graphs showing a receiver operating characteristic (ROC) from 42 variables that appear at least 10% of the times in 300 LASSO survival models.

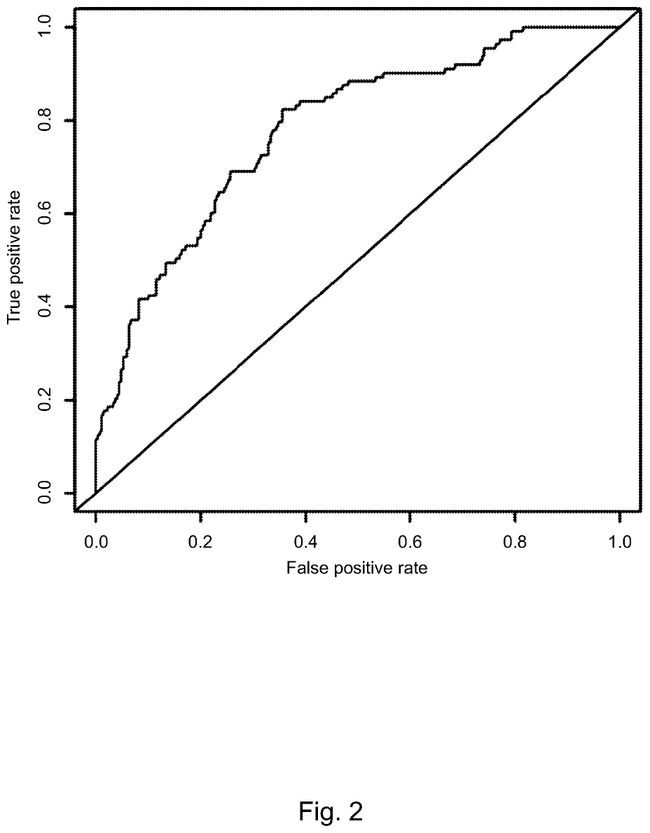

FIG. 3 contains a graph showing variables that appear at least 10% of times in 300 models generated using a double-loop cross validation (DLCV) method.

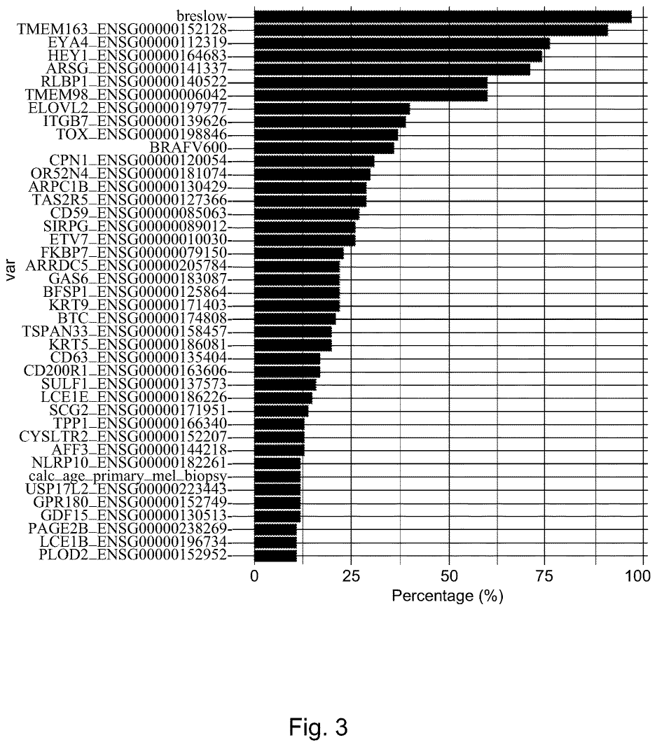

FIG. 4 contains a table showing exemplary polypeptides having altered levels in melanoma cells from PCMs that are likely to metastasize.

FIG. 5 contains a graph showing a receiver operating characteristic (ROC) from 13 variables that appear at least 50% of the time.

FIG. 6 contains a graph showing variables that appear at least 10% of times in 300 models generated using a double-loop cross validation (DLCV) method.

FIG. 7 contains a table showing exemplary polypeptides having altered levels in melanoma cells from PCMs that are likely to metastasize.

FIG. 8 shows changes in levels of TMEM163 polypeptide and mRNA encoding a TMEM163 polypeptide in humans relative to low levels of expression in control (non-metastatic) melanoma. Low level of expression (“low”) is defined here as the smallest dataset number larger than 1.5 times the interquartile range (IQR) below the first quartile. High level of expression (“high”) is defined as the largest dataset number smaller than 1.5 times IQR above the third quartile. “High risk” refers to high risk melanoma, i.e., melanoma that metastasized.

FIG. 9 shows changes in levels of HEY1 polypeptide and mRNA encoding a HEY1 polypeptide in humans relative to low levels of expression in control (non-metastatic) melanoma. Low level of expression (“low”) is defined here as the smallest dataset number larger than 1.5 times the interquartile range (IQR) below the first quartile. High level of expression (“high”) is defined as the largest dataset number smaller than 1.5 times IQR above the third quartile. “High risk” refers to high risk melanoma, i.e., melanoma that metastasized.

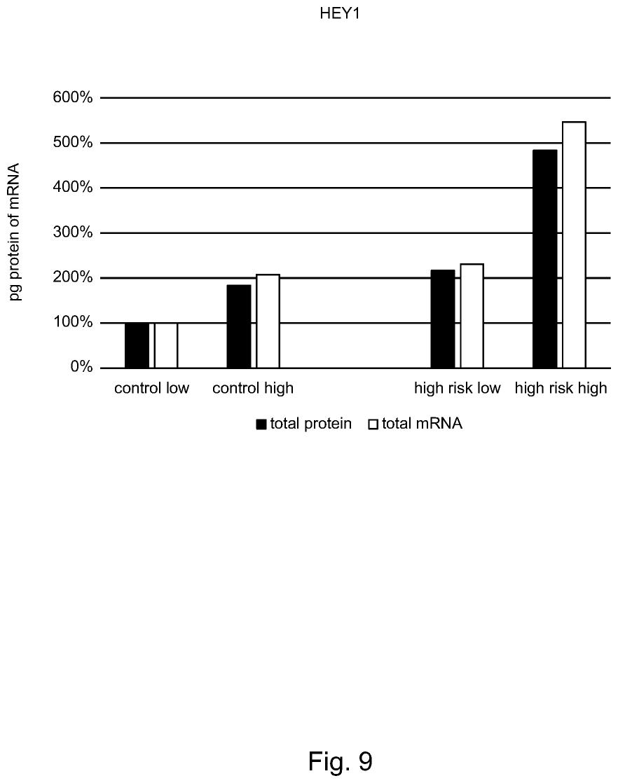

FIG. 10 shows changes in levels of EYA4 polypeptide and mRNA encoding a EYA4 polypeptide in humans relative to low levels of expression in control (non-metastatic) melanoma. Low level of expression (“low”) is defined here as the smallest dataset number larger than 1.5 times the interquartile range (IQR) below the first quartile. High level of expression (“high”) is defined as the largest dataset number smaller than 1.5 times IQR above the third quartile. “High risk” refers to high risk melanoma, i.e., melanoma that metastasized.

FIG. 11 shows changes in levels of TMEM98 polypeptide and mRNA encoding a TMEM98 polypeptide in humans relative to low levels of expression in control (non-metastatic) melanoma. Low level of expression (“low”) is defined here as the smallest dataset number larger than 1.5 times the interquartile range (IQR) below the first quartile. High level of expression (“high”) is defined as the largest dataset number smaller than 1.5 times IQR above the third quartile. “High risk” refers to high risk melanoma, i.e., melanoma that metastasized.

FIG. 12 shows changes in levels of RLBP1 polypeptide and mRNA encoding a RLBP1 polypeptide in humans relative to low levels of expression in control (non-metastatic) melanoma. Low level of expression (“low”) is defined here as the smallest dataset number larger than 1.5 times the interquartile range (IQR) below the first quartile. High level of expression (“high”) is defined as the largest dataset number smaller than 1.5 times IQR above the third quartile. “High risk” refers to high risk melanoma, i.e., melanoma that metastasized.

FIG. 13 shows changes in levels of ELOVL2 polypeptide and mRNA encoding a ELOVL2 polypeptide in humans relative to low levels of expression in control (non-metastatic) melanoma. Low level of expression (“low”) is defined here as the smallest dataset number larger than 1.5 times the interquartile range (IQR) below the first quartile. High level of expression (“high”) is defined as the largest dataset number smaller than 1.5 times IQR above the third quartile. “High risk” refers to high risk melanoma, i.e., melanoma that metastasized.

FIG. 14 shows changes in levels of SERPINE2 polypeptide and mRNA encoding a SERPINE2 polypeptide in humans relative to low levels of expression in control (non-metastatic) melanoma. Low level of expression (“low”) is defined here as the smallest dataset number larger than 1.5 times the interquartile range (IQR) below the first quartile. High level of expression (“high”) is defined as the largest dataset number smaller than 1.5 times IQR above the third quartile. “High risk” refers to high risk melanoma, i.e., melanoma that metastasized.

FIG. 15 shows changes in levels of ITGB3 polypeptide and mRNA encoding a ITGB3 polypeptide in humans relative to low levels of expression in control (non-metastatic) melanoma. Low level of expression (“low”) is defined here as the smallest dataset number larger than 1.5 times the interquartile range (IQR) below the first quartile. High level of expression (“high”) is defined as the largest dataset number smaller than 1.5 times IQR above the third quartile. “High risk” refers to high risk melanoma, i.e., melanoma that metastasized.

FIG. 16 shows changes in levels of IGF1R polypeptide and mRNA encoding a IGF1R polypeptide in humans relative to low levels of expression in control (non-metastatic) melanoma. Low level of expression (“low”) is defined here as the smallest dataset number larger than 1.5 times the interquartile range (IQR) below the first quartile. High level of expression (“high”) is defined as the largest dataset number smaller than 1.5 times IQR above the third quartile. “High risk” refers to high risk melanoma, i.e., melanoma that metastasized.

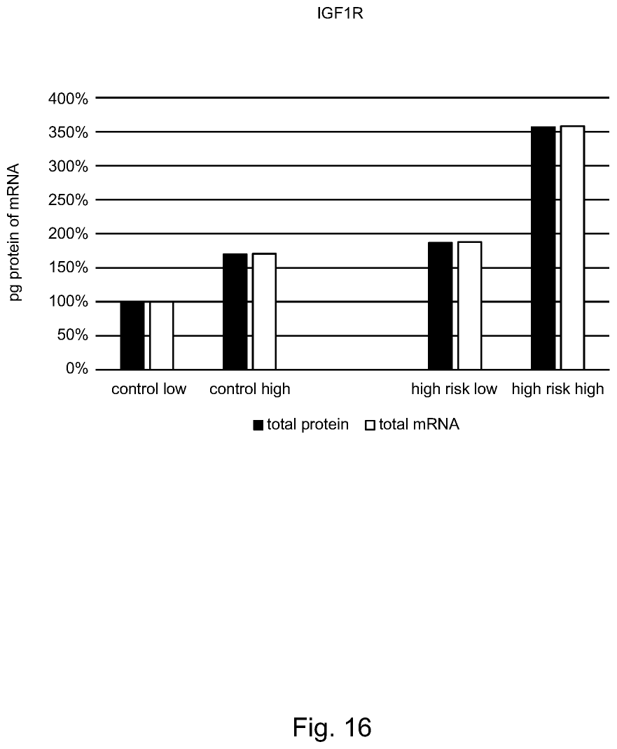

FIG. 17 shows changes in levels of CD200R1 polypeptide and mRNA encoding a CD200R1 polypeptide in humans relative to low levels of expression in control (non-metastatic) melanoma. Low level of expression (“low”) is defined here as the smallest dataset number larger than 1.5 times the interquartile range (IQR) below the first quartile. High level of expression (“high”) is defined as the largest dataset number smaller than 1.5 times IQR above the third quartile. “High risk” refers to high risk melanoma, i.e., melanoma that metastasized.

FIG. 18 shows changes in levels of SIRPG polypeptide and mRNA encoding a SIRPG polypeptide in humans relative to low levels of expression in control (non-metastatic) melanoma. Low level of expression (“low”) is defined here as the smallest dataset number larger than 1.5 times the interquartile range (IQR) below the first quartile. High level of expression (“high”) is defined as the largest dataset number smaller than 1.5 times IQR above the third quartile. “High risk” refers to high risk melanoma, i.e., melanoma that metastasized.

FIG. 19 shows changes in levels of BTC polypeptide and mRNA encoding a BTC polypeptide in humans relative to low levels of expression in control (non-metastatic) melanoma. Low level of expression (“low”) is defined here as the smallest dataset number larger than 1.5 times the interquartile range (IQR) below the first quartile. High level of expression (“high”) is defined as the largest dataset number smaller than 1.5 times IQR above the third quartile. “High risk” refers to high risk melanoma, i.e., melanoma that metastasized.

FIG. 20 shows changes in levels of OR52N4 polypeptide and mRNA encoding a OR52N4 polypeptide in humans relative to low levels of expression in control (non-metastatic) melanoma. Low level of expression (“low”) is defined here as the smallest dataset number larger than 1.5 times the interquartile range (IQR) below the first quartile. High level of expression (“high”) is defined as the largest dataset number smaller than 1.5 times IQR above the third quartile. “High risk” refers to high risk melanoma, i.e., melanoma that metastasized.

FIG. 21 shows changes in levels of ETV7 polypeptide and mRNA encoding a ETV7 polypeptide in humans relative to low levels of expression in control (non-metastatic) melanoma. Low level of expression (“low”) is defined here as the smallest dataset number larger than 1.5 times the interquartile range (IQR) below the first quartile. High level of expression (“high”) is defined as the largest dataset number smaller than 1.5 times IQR above the third quartile. “High risk” refers to high risk melanoma, i.e., melanoma that metastasized.

FIG. 22 shows changes in levels of TOX polypeptide and mRNA encoding a TOX polypeptide in humans relative to low levels of expression in control (non-metastatic) melanoma. Low level of expression (“low”) is defined here as the smallest dataset number larger than 1.5 times the interquartile range (IQR) below the first quartile. High level of expression (“high”) is defined as the largest dataset number smaller than 1.5 times IQR above the third quartile. “High risk” refers to high risk melanoma, i.e., melanoma that metastasized.

FIG. 23 shows changes in levels of ARSG polypeptide and mRNA encoding an ARSG polypeptide in humans relative to low levels of expression in control (non-metastatic) melanoma. Low level of expression (“low”) is defined here as the smallest dataset number larger than 1.5 times the interquartile range (IQR) below the first quartile. High level of expression (“high”) is defined as the largest dataset number smaller than 1.5 times IQR above the third quartile. “High risk” refers to high risk melanoma, i.e., melanoma that metastasized.

FIG. 24 shows changes in levels of ITGB7 polypeptide and mRNA encoding a ITGB7 polypeptide in humans relative to low levels of expression in control (non-metastatic) melanoma. Low level of expression (“low”) is defined here as the smallest dataset number larger than 1.5 times the interquartile range (IQR) below the first quartile. High level of expression (“high”) is defined as the largest dataset number smaller than 1.5 times IQR above the third quartile. “High risk” refers to high risk melanoma, i.e., melanoma that metastasized.

FIG. 25 shows changes in levels of CDKN2A polypeptide and mRNA encoding a CDKN2A polypeptide in humans relative to low levels of expression in control (non-metastatic) melanoma. Low level of expression (“low”) is defined here as the smallest dataset number larger than 1.5 times the interquartile range (IQR) below the first quartile. High level of expression (“high”) is defined as the largest dataset number smaller than 1.5 times IQR above the third quartile. “High risk” refers to high risk melanoma, i.e., melanoma that metastasized.

FIG. 26 shows changes in levels of DCD polypeptide and mRNA encoding a DCD polypeptide in humans relative to low levels of expression in control (non-metastatic) melanoma. Low level of expression (“low”) is defined here as the smallest dataset number larger than 1.5 times the interquartile range (IQR) below the first quartile. High level of expression (“high”) is defined as the largest dataset number smaller than 1.5 times IQR above the third quartile. “High risk” refers to high risk melanoma, i.e., melanoma that metastasized.

### DETAILED DESCRIPTION

This document provides methods and materials that can be used to determine whether or not a melanoma (e.g., a PCM) is likely to metastasize (e.g., likely to metastasize to one or more lymph nodes). For example, a sample (e.g., a tissue sample containing one or more melanoma cells) obtained from a mammal having melanoma (e.g., PCM) can be assessed for the molecular signature of the melanoma to determine whether or not the melanoma is likely to metastasize. In some cases, the methods and materials provided herein also can include treating a mammal having melanoma (e.g., PCM) where one or more cancer treatments are selected based, at least in part, on whether or not the melanoma is likely to metastasize.

A mammal (e.g., a human) having melanoma (e.g., PCM) can be assessed to determine whether or not the melanoma is likely to metastasize (e.g., likely to metastasize to one or more lymph nodes) by detecting the molecular signature of the melanoma. For example, a sample (e.g., a tissue sample containing one or more melanoma cells) obtained from a mammal having melanoma (e.g., PCM) can be assessed for (a) the presence or absence of altered levels (e.g., increased levels or decreased levels) of 11 or more (e.g., eleven, twelve, thirteen, or fourteen) polypeptides (or mRNA encoding such polypeptides) and/or (b) the presence or absence of one or more (e.g., one, two, or three) mutations in one or more polypeptides (e.g., a BRAF polypeptide and a NRAS polypeptide) to determine whether or not that melanoma is likely to metastasize. As described herein, the presence of an increased level of a TMEM163 polypeptide (or mRNA encoding the TMEM163 polypeptide), an increased level of a HEY1 polypeptide (or mRNA encoding the HEY1 polypeptide), an increased level of an EYA4 polypeptide (or mRNA encoding the EYA4 polypeptide), an increased level of a TMEM98 polypeptide (or mRNA encoding the TMEM98 polypeptide), an increased level of a RLBP1 polypeptide (or mRNA encoding the RLBP1 polypeptide), an increased level of an ELOVL2 polypeptide (or mRNA encoding the ELOVL2 polypeptide), an increased level of a SERPINE2 polypeptide (or mRNA encoding a SERPINE2 polypeptide), an increased level of an ITGB3 polypeptide (or mRNA encoding an ITGB3 polypeptide), an increased level of an IGF1R polypeptide (or mRNA encoding an IGF1R polypeptide), a decreased level of a CD200R1 polypeptide (or mRNA encoding the CD200R1 polypeptide), a decreased level of a SIRPG polypeptide (or mRNA encoding the SIRPG polypeptide), a decreased level of a BTC polypeptide (or mRNA encoding the BTC polypeptide), a decreased level of an OR52N4 polypeptide (or mRNA encoding the OR52N4 polypeptide), a decreased level of an ETV7 polypeptide (or mRNA encoding the ETV7 polypeptide), a decreased level of a TOX polypeptide (or mRNA encoding the TOX polypeptide), a decreased level of an ARSG polypeptide (or mRNA encoding the ARSG polypeptide), a decreased level of an ITGB7 polypeptide (or mRNA encoding the ITGB7 polypeptide), a decreased level of a CDKN2A polypeptide (or mRNA encoding a CDKN2A polypeptide), and a decreased level of a DCD polypeptide (or mRNA encoding a DCD polypeptide) in melanoma cells of a mammal having melanoma (e.g., PCM) can be used to identify that melanoma as being likely to metastasize to one or more lymph nodes. Also as demonstrated herein, the presence of one or more mutations in a BRAF polypeptide and/or the presence of one or more mutations in a NRAS polypeptide in melanoma cells of a mammal having melanoma (e.g., PCM) can be used to identify that melanoma as being likely to metastasize to one or more lymph nodes.

Any appropriate mammal having melanoma (e.g., PCM) can be assessed and/or treated as described herein. Examples of mammals that can have melanoma (e.g., PCM) and can be assessed and/or treated as described herein include, without limitation, humans, non-human primates (e.g., monkeys), dogs, cats, horses, cows, pigs, sheep, mice, and rats. In some cases, a human having melanoma (e.g., PCM) can be assessed and/or treated as described herein.

When assessing and/or treating a mammal (e.g., a human) having melanoma as described herein, the melanoma can be any type of melanoma. A melanoma can be any stage of melanoma (e.g., stage 0, stage I, stage II, stage III, or stage IV). A melanoma can be at any location on a mammal (e.g., a human) being assessed and/or treated as described herein. In some cases, a melanoma can be at a location on a mammal's body that has been exposed to ultraviolet (UV) radiation. For example, a melanoma can be at a location on a mammal's body that has had exposure to the sun. For example, a melanoma can be at a location on a mammal's body that has had exposure to a tanning lamp and/or a tanning bed. Examples of locations on a mammal's body where a melanoma that can be assessed and/or treated as described herein include, without limitation, abdomen, back, legs, arms, neck, face, ears, scalp, dorsum and soles of the feet, dorsum and palms of the hands, fingers, fingernail beds, toes, and/or toenail beds.

In some cases, the methods described herein can include identifying a mammal (e.g., a human) as having melanoma (e.g., PCM). Any appropriate method can be used to identify a mammal as having melanoma (e.g., PCM). For example, physical examination (e.g., to examine the skin to look for signs that may indicate melanoma), imaging techniques (e.g., X-rays, computerized tomography (CT) scanning, and positron emission tomography (PET)), laboratory examination (e.g., of a skin sample obtained by biopsy), blood sample (liquid biopsy), urine sample and/or electrical impedance spectroscopy can be used to identify a mammal (e.g., a human) as having melanoma (e.g., PCM). In some cases, a mammal can be identified as having melanoma (e.g., a low-risk melanoma such as a PCM that is identified as not being likely to metastasize as described herein) without having been subjected to a sentinel lymph node biopsy (SLNb). In some cases, a mammal can be identified as having melanoma (e.g., a low-risk melanoma such as a PCM that is identified as not being likely to metastasize as described herein) and having a negative SLNb.

In some cases, a molecular signature used to determine whether or not a melanoma (e.g., a PCM) is likely to metastasize (e.g., likely to metastasize to one or more lymph nodes) can include an altered level (e.g., an increased level or a decreased level) of 11 or more polypeptides (or mRNA encoding such polypeptides). For example, a sample (e.g., a tissue sample containing one or more melanoma cells) obtained from a mammal having melanoma (e.g., PCM) can be assessed for the presence or absence of an altered level (e.g., an increased level or a decreased level) of 11 or more polypeptides (or mRNA encoding such polypeptides) in a sample (e.g., a tissue sample containing one or more melanoma cells) obtained from the mammal to determine whether or not the melanoma is likely to metastasize (e.g., likely to metastasize to one or more lymph nodes). In some cases, a melanoma (e.g., PCM) can be identified as likely to metastasize (e.g., likely to metastasize to one or more lymph nodes) based, at least in part, on a molecular signature that includes the presence of an altered level of 11 or more polypeptides (or mRNA encoding such polypeptides) in a sample (e.g., a tissue sample containing one or more melanoma cells) obtained from a mammal having the melanoma. In some cases, an altered level can be an increased level of one or more polypeptides (or mRNA encoding such polypeptides) in a sample (e.g., a tissue sample containing one or more melanoma cells) obtained from a mammal having melanoma (e.g., PCM). The term “increased level” as used herein with respect to a level of a polypeptide (or mRNA encoding that polypeptide) in a sample refers to any level that is higher than a reference level of the polypeptide (or mRNA). In some cases, an altered level can be a decreased level of a polypeptide (or mRNA encoding that polypeptide) in a sample (e.g., a tissue sample containing one or more melanoma cells) obtained from a mammal having melanoma (e.g., PCM). The term “decreased level” as used herein with respect to a level of a polypeptide (or mRNA encoding that polypeptide) in a sample refers to any level that is lower than a reference level of the polypeptide (or mRNA). The term “reference level” as used herein with respect to a level of a polypeptide (or mRNA) refers to the level of the polypeptide (or mRNA) typically observed in a control sample. Control samples are samples obtained from mammals having melanoma at the time of initial diagnosis and having a SLNb performed within 90 days of the diagnosis, SLNb biopsy showed no evidence of metastasis and there was no melanoma relapse within 90 days of diagnosis. It will be appreciated that levels of polypeptides (or mRNAs) from comparable samples are used when determining whether or not a particular level is an altered level of a polypeptide (or mRNA).

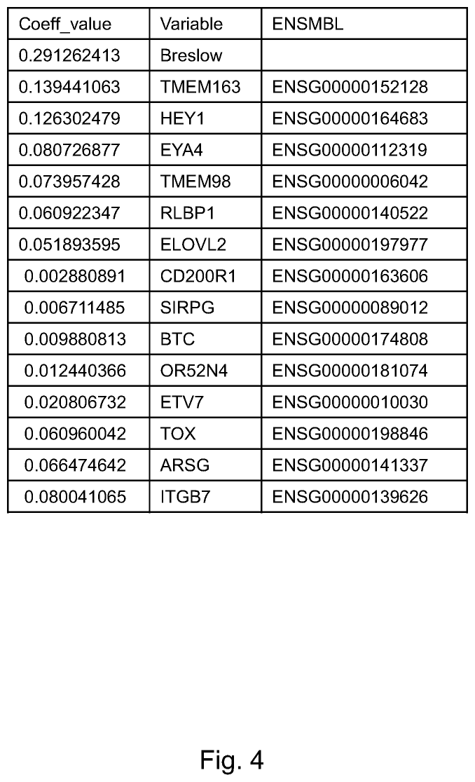

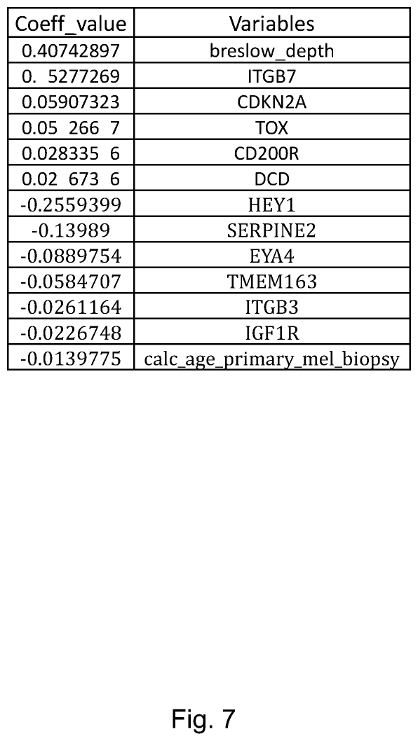

A melanoma (e.g., PCM) that is likely to metastasize (e.g., likely to metastasize to one or more lymph nodes) can have the presence of an altered level of any appropriate polypeptides (or mRNAs encoding the polypeptides) in a sample (e.g., a tissue sample containing one or more melanoma cells) obtained from a mammal having the melanoma. Examples of polypeptides that can be assessed to determine if an altered level is present or absent within a sample (e.g., a tissue sample containing one or more melanoma cells) obtained from a mammal having melanoma (e.g., PCM) as described herein include, without limitation, a TMEM163 polypeptide, a HEY1 polypeptide, an EYA4 polypeptide, a TMEM98 polypeptide, a RLBP1 polypeptide, an ELOVL2 polypeptide, a SERPINE2 polypeptide, an ITGB3 polypeptide, an IGF1R polypeptide, a CD200R1 polypeptide, a SIRPG polypeptide, a BTC polypeptide, an OR52N4 polypeptide, an ETV7 polypeptide, a TOX polypeptide, an ARSG polypeptide, an ITGB7 polypeptide, a CDKN2A polypeptide, and a DCD polypeptide. Examples of mRNA that can be assessed to determine if an altered level is present or absent within a sample (e.g., a tissue sample containing one or more melanoma cells) obtained from a mammal having melanoma (e.g., PCM) as described herein include, without limitation, mRNAs encoding a TMEM163 polypeptide, a HEY1 polypeptide, an EYA4 polypeptide, a TMEM98 polypeptide, a RLBP1 polypeptide, an ELOVL2 polypeptide, a SERPINE2 polypeptide, an ITGB3 polypeptide, an IGF1R polypeptide, a CD200R1 polypeptide, a SIRPG polypeptide, a BTC polypeptide, an OR52N4 polypeptide, an ETV7 polypeptide, a TOX polypeptide, an ARSG polypeptide, an ITGB7 polypeptide, a CDKN2A polypeptide, or a DCD polypeptide. In some cases, a polypeptide that can be assessed to determine if an altered level is present or absent within a sample (e.g., a tissue sample containing one or more melanoma cells) obtained from a mammal having melanoma (e.g., PCM) can be as listed in FIG. 4 or FIG. 7. In some cases, an mRNA encoding a polypeptide as listed in FIG. 4 or FIG. 7 can be assessed to determine if an altered level is present or absent within a sample (e.g., a tissue sample containing one or more melanoma cells) obtained from a mammal having melanoma (e.g., PCM). In some cases, a polypeptide that can be assessed to determine if an increased level is present or absent within a sample (e.g., a tissue sample containing one or more melanoma cells) obtained from a mammal having melanoma (e.g., PCM) can be as listed in Table 1, Table 2, and/or Table 3. In some cases, an mRNA encoding a polypeptide as listed in Table 1, Table 2, and/or Table 3 can be assessed to determine if an altered level is present or absent within a sample (e.g., a tissue sample containing one or more melanoma cells) obtained from a mammal having melanoma (e.g., PCM).

In some cases, a melanoma (e.g., PCM) is not assessed for an altered (e.g., an increased or a decreased) level of a TGFBR1 polypeptide (and an mRNA encoding a TGFBR1 polypeptide), a SERPINE2 polypeptide (and an mRNA encoding a SERPINE2 polypeptide), a CXCL8 polypeptide (and an mRNA encoding a CXCL8 polypeptide), a GDF15 polypeptide (and an mRNA encoding a GDF15 polypeptide), a PLAT polypeptide (and an mRNA encoding a PLAT polypeptide), a MLANA polypeptide (and an mRNA encoding a MLANA polypeptide), a ITGB3 polypeptide (and an mRNA encoding a ITGB3 polypeptide), and/or a LOXL4 polypeptide (and an mRNA encoding a LOXL4 polypeptide).

In some cases, a melanoma (e.g., PCM) that is likely to metastasize (e.g., likely to metastasize to one or more lymph nodes) can lack an increased level of a TGFBR1 polypeptide (or an mRNA encoding a TGFBR1 polypeptide), an increased level of a SERPINE2 polypeptide (or an mRNA encoding a SERPINE2 polypeptide), an increased level of a CXCL8 polypeptide (or an mRNA encoding a CXCL8 polypeptide), an increased level of a GDF15 polypeptide (or an mRNA encoding a GDF15 polypeptide), an increased level of a PLAT polypeptide (or an mRNA encoding a PLAT polypeptide), an increased level of a MLANA polypeptide (or an mRNA encoding a MLANA polypeptide), an increased level of a ITGB3 polypeptide (or an mRNA encoding a ITGB3 polypeptide), and/or an increased level of a LOXL4 polypeptide (or an mRNA encoding a LOXL4 polypeptide).

In some cases, an altered level of a polypeptide (or mRNA) can be an increased level of a TMEM163 polypeptide (or an increased level of an mRNA encoding a TMEM163 polypeptide). Examples of TMEM163 polypeptides and nucleic acid sequences encoding a TMEM163 polypeptide include, without limitation, those set forth in the National Center for Biotechnology Information (NCBI) databases at, for example, accession no. NP_112185 (version NP_112185.1). For example, an increased level of a TMEM163 polypeptide (or an mRNA encoding a TMEM163 polypeptide) can be any level that is higher than a reference level of the TMEM163 polypeptide (or a reference level of an mRNA encoding a TMEM163 polypeptide). In some cases, a reference level of a TMEM163 polypeptide in humans can be a level of a TMEM163 polypeptide in a sample (e.g., a tissue sample containing one or more melanoma cells) obtained from the human of from about 0.2 picograms (pg)/melanoma cell to about 0.4 pg/melanoma cell. For example, an increased level of a TMEM163 polypeptide can be at least 5% (e.g., about 5%, about 10%, about 20%, about 30%, about 40%, about 50%, about 60%, about 70%, about 80%, about 90%, about 95%, or more) higher than a reference level of a TMEM163 polypeptide. For example, an increased level of a TMEM163 polypeptide can be a level that is at least 2 (e.g., at least 5, at least 10, at least 15, at least 20, at least 25, at least 35, or at least 50) fold greater than a reference level of a TMEM163 polypeptide. In some cases, a reference level of an mRNA encoding a TMEM163 polypeptide in humans can be a level of such mRNA that is from about 0.03 pg/melanoma cell to about 0.00 pg/melanoma cell. For example, an increased level of an mRNA encoding a TMEM163 polypeptide can be a level that is at least 5% (e.g., about 5%, about 10%, about 20%, about 30%, about 40%, about 50%, about 60%, about 70%, about 80%, about 90%, about 95%, or more) higher than a reference level of mRNA encoding a TMEM163 polypeptide. For example, an increased level of an mRNA encoding a TMEM163 polypeptide can be a level that is at least 2 (e.g., at least 5, at least 10, at least 15, at least 20, at least 25, at least 35, or at least 50) fold greater than a reference level of mRNA encoding a TMEM163 polypeptide.

In some cases, an altered level of a polypeptide (or mRNA) can be an increased level of a HEY1 polypeptide (or an increased level of an mRNA encoding a HEY1 polypeptide). Examples of HEY1 polypeptides and nucleic acid sequences encoding a HEY1 polypeptide include, without limitation, those set forth in the NCBI databases at, for example, accession no. CAB75715 (version CAB75715.1). For example, an increased level of a HEY1 polypeptide (or an mRNA encoding a HEY1 polypeptide) can be any level that is higher than a reference level of the HEY1 polypeptide (or a reference level of an mRNA encoding a HEY1 polypeptide). In some cases, a reference level of a HEY1 polypeptide in humans can be a level of a HEY1 polypeptide in a sample (e.g., a tissue sample containing one or more melanoma cells) obtained from the human of from about 0.6 pg/melanoma cell to about 1.1 pg/melanoma cell. For example, an increased level of a HEY1 polypeptide can be at least 5% (e.g., about 5%, about 10%, about 20%, about 30%, about 40%, about 50%, about 60%, about 70%, about 80%, about 90%, about 95%, or more) higher than a reference level of a HEY1 polypeptide. For example, an increased level of a HEY1 polypeptide can be a level that is at least 2 (e.g., at least 5, at least 10, at least 15, at least 20, at least 25, at least 35, or at least 50) fold greater than a reference level of a HEY1 polypeptide. In some cases, a reference level of an mRNA encoding a HEY1 polypeptide in humans can be a level of such mRNA that is from about 0.13 pg/melanoma cell to about 0.27 pg/melanoma cell. For example, an increased level of mRNA encoding a HEY1 polypeptide can be a level that is at least 5% (e.g., about 5%, about 10%, about 20%, about 30%, about 40%, about 50%, about 60%, about 70%, about 80%, about 90%, about 95%, or more) higher than a reference level of mRNA encoding a HEY1 polypeptide. For example, an increased level of mRNA encoding a HEY1 polypeptide can be a level that is at least 2 (e.g., at least 5, at least 10, at least 15, at least 20, at least 25, at least 35, or at least 50) fold greater than a reference level of mRNA encoding a HEY1 polypeptide.

In some cases, an altered level of a polypeptide (or mRNA) can be an increased level of an EYA4 polypeptide (or an increased level of an mRNA encoding an EYA4 polypeptide). Examples of EYA4 polypeptides and nucleic acid sequences encoding an EYA4 polypeptide include, without limitation, those set forth in the NCBI databases at, for example, accession no. CAA76636 (version CAA76636.1). For example, an increased level of an EYA4 polypeptide (or an mRNA encoding an EYA4 polypeptide) can be any level that is higher than a reference level of the EYA4 polypeptide (or a reference level of an mRNA encoding an EYA4 polypeptide). In some cases, a reference level of an EYA4 polypeptide in humans can be a level of an EYA4 polypeptide in a sample (e.g., a tissue sample containing one or more melanoma cells) obtained from the human of from about 2.0 pg/melanoma cell to about 2.7 pg/melanoma cell. For example, an increased level of an EYA4 polypeptide can be at least 5% (e.g., about 5%, about 10%, about 20%, about 30%, about 40%, about 50%, about 60%, about 70%, about 80%, about 90%, about 95%, or more) higher than a reference level of an EYA4 polypeptide. For example, an increased level of an EYA4 polypeptide can be a level that is at least 2 (e.g., at least 5, at least 10, at least 15, at least 20, at least 25, at least 35, or at least 50) fold greater than a reference level of an EYA4 polypeptide. In some cases, a reference level of an mRNA encoding an EYA4 polypeptide in humans can be a level of such mRNA that is from about 0.4 pg/melanoma cell to about 5.47 pg/melanoma cell. For example, an increased level of mRNA encoding an EYA4 polypeptide can be a level that is at least 5% (e.g., about 5%, about 10%, about 20%, about 30%, about 40%, about 50%, about 60%, about 70%, about 80%, about 90%, about 95%, or more) higher than a reference level of mRNA encoding an EYA4 polypeptide. For example, an increased level of mRNA encoding an EYA4 polypeptide can be a level that is at least 2 (e.g., at least 5, at least 10, at least 15, at least 20, at least 25, at least 35, or at least 50) fold greater than a reference level of mRNA encoding an EYA4 polypeptide.

In some cases, an altered level of a polypeptide (or mRNA) can be an increased level of a TMEM98 polypeptide (or an increased level of an mRNA encoding a TMEM98 polypeptide). Examples of TMEM98 polypeptides and nucleic acid sequences encoding a TMEM98 polypeptide include, without limitation, those set forth in the NCBI databases at, for example, accession no. NP_001028676 (version NP_001028676.1) and accession no. NP_056359 (version NP_056359.2). For example, an increased level of a TMEM98 polypeptide (or an mRNA encoding a TMEM98 polypeptide) can be any level that is higher than a reference level of the TMEM98 polypeptide (or a reference level of an mRNA encoding a TMEM98 polypeptide). In some cases, a reference level of a TMEM98 polypeptide in humans can be a level of a TMEM98 polypeptide in a sample (e.g., a tissue sample containing one or more melanoma cells) obtained from the human of from about 0.6 pg/melanoma cell to about 1.1 pg/melanoma cell. For example, an increased level of a TMEM98 polypeptide can be at least 5% (e.g., about 5%, about 10%, about 20%, about 30%, about 40%, about 50%, about 60%, about 70%, about 80%, about 90%, about 95%, or more) higher than a reference level of a TMEM98 polypeptide. For example, an increased level of a TMEM98 polypeptide can be a level that is at least 2 (e.g., at least 5, at least 10, at least 15, at least 20, at least 25, at least 35, or at least 50) fold greater than a reference level of a TMEM98 polypeptide. In some cases, a reference level of an mRNA encoding a TMEM98 polypeptide in humans can be a level of such mRNA that is from about 0.13 pg/melanoma cell to about 0.27 pg/melanoma cell. For example, an increased level of mRNA encoding a TMEM98 polypeptide can be a level that is at least 5% (e.g., about 5%, about 10%, about 20%, about 30%, about 40%, about 50%, about 60%, about 70%, about 80%, about 90%, about 95%, or more) higher than a reference level of mRNA encoding a TMEM98 polypeptide. For example, an increased level of mRNA encoding a TMEM98 polypeptide can be a level that is at least 2 (e.g., at least 5, at least 10, at least 15, at least 20, at least 25, at least 35, or at least 50) fold greater than a reference level of mRNA encoding a TMEM98 polypeptide.

In some cases, an altered level of a polypeptide (or mRNA) can be an increased level of a RLBP1 polypeptide (or an increased level of an mRNA encoding a RLBP1 polypeptide). Examples of RLBP1 polypeptides and nucleic acid sequences encoding a RLBP1 polypeptide include, without limitation, those set forth in the NCBI databases at, for example, accession no. NP_000317 (version NP_000317.1) and accession no. EAX02038 (version EAX02038.1). For example, an increased level of a RLBP1 polypeptide (or an mRNA encoding a RLBP1 polypeptide) can be any level that is higher than a reference level of the RLBP1 polypeptide (or a reference level of an mRNA encoding a RLBP1 polypeptide). In some cases, a reference level of a RLBP1 polypeptide in humans can be a level of a RLBP1 polypeptide in a sample (e.g., a tissue sample containing one or more melanoma cells) obtained from the human of from about 0.4 pg/melanoma cell to about 0.6 pg/melanoma cell. For example, an increased level of a RLBP1 polypeptide can be at least 5% (e.g., about 5%, about 10%, about 20%, about 30%, about 40%, about 50%, about 60%, about 70%, about 80%, about 90%, about 95%, or more) higher than a reference level of a RLBP1 polypeptide. For example, an increased level of a RLBP1 polypeptide can be a level that is at least 2 (e.g., at least 5, at least 10, at least 15, at least 20, at least 25, at least 35, or at least 50) fold greater than a reference level of a RLBP1 polypeptide. In some cases, a reference level of an mRNA encoding a RLBP1 polypeptide in humans can be a level of such mRNA that is from about 0.06 pg/melanoma cell to about 0.1 pg/melanoma cell. For example, an increased level of mRNA encoding a RLBP1 polypeptide can be a level that is at least 5% (e.g., about 5%, about 10%, about 20%, about 30%, about 40%, about 50%, about 60%, about 70%, about 80%, about 90%, about 95%, or more) higher than a reference level of mRNA encoding a RLBP1 polypeptide. For example, an increased level of mRNA encoding a RLBP1 polypeptide can be a level that is at least 2 (e.g., at least 5, at least 10, at least 15, at least 20, at least 25, at least 35, or at least 50) fold greater than a reference level of mRNA encoding a RLBP1 polypeptide.

In some cases, an altered level of a polypeptide (or mRNA) can be an increased level of an ELOVL2 polypeptide (or an increased level of an mRNA encoding an ELOVL2 polypeptide). Examples of ELOVL2 polypeptides and nucleic acid sequences encoding an ELOVL2 polypeptide include, without limitation, those set forth in the NCBI databases at, for example, accession no. NP_060240 (version NP_060240.3) and accession no. AAM00193 (version AAM00193.1). For example, an increased level of an ELOVL2 polypeptide (or an mRNA encoding an ELOVL2 polypeptide) can be any level that is higher than a reference level of the ELOVL2 polypeptide (or a reference level of an mRNA encoding an ELOVL2 polypeptide). In some cases, a reference level of an ELOVL2 polypeptide in humans can be a level of an ELOVL2 polypeptide in a sample (e.g., a tissue sample containing one or more melanoma cells) obtained from the human of from about 0.4 pg/melanoma cell to about 0.9 pg/melanoma cell. For example, an increased level of an ELOVL2 polypeptide can be at least 5% (e.g., about 5%, about 10%, about 20%, about 30%, about 40%, about 50%, about 60%, about 70%, about 80%, about 90%, about 95%, or more) higher than a reference level of an ELOVL2 polypeptide. For example, an increased level of an ELOVL2 polypeptide can be a level that is at least 2 (e.g., at least 5, at least 10, at least 15, at least 20, at least 25, at least 35, or at least 50) fold greater than a reference level of an ELOVL2 polypeptide. In some cases, a reference level of an mRNA encoding an ELOVL2 polypeptide in humans can be a level of such mRNA that is from about 0.14 pg/melanoma cell to about 0.35 pg/melanoma cell. For example, an increased level of mRNA encoding an ELOVL2 polypeptide can be a level that is at least 5% (e.g., about 5%, about 10%, about 20%, about 30%, about 40%, about 50%, about 60%, about 70%, about 80%, about 90%, about 95%, or more) higher than a reference level of mRNA encoding an ELOVL2 polypeptide. For example, an increased level of mRNA encoding an ELOVL2 polypeptide can be a level that is at least 2 (e.g., at least 5, at least 10, at least 15, at least 20, at least 25, at least 35, or at least 50) fold greater than a reference level of mRNA encoding an ELOVL2 polypeptide.

In some cases, an altered level of a polypeptide (or mRNA) can be an increased level of a SERPINE2 polypeptide (or an increased level of an mRNA encoding a SERPINE2 polypeptide). Examples of SERPINE2 polypeptides and nucleic acid sequences encoding a SERPINE2 polypeptide include, without limitation, those set forth in the NCBI databases at, for example, accession no. AAH42628 (version AAH42628.1). For example, an increased level of a SERPINE2 polypeptide (or an mRNA encoding a SERPINE2 polypeptide) can be any level that is higher than a reference level of the SERPINE2 polypeptide (or a reference level of an mRNA encoding a SERPINE2 polypeptide). In some cases, a reference level of a SERPINE2 polypeptide in humans can be a level of a SERPINE2 polypeptide in a sample (e.g., a tissue sample containing one or more melanoma cells) obtained from the human of from about 2.0 pg/melanoma cell to about 27.0 pg/melanoma cell. For example, an increased level of a SERPINE2 polypeptide can be at least 5% (e.g., about 5%, about 10%, about 20%, about 30%, about 40%, about 50%, about 60%, about 70%, about 80%, about 90%, about 95%, or more) higher than a reference level of a SERPINE2 polypeptide. For example, an increased level of a SERPINE2 polypeptide can be a level that is at least 2 (e.g., at least 5, at least 10, at least 15, at least 20, at least 25, at least 35, or at least 50) fold greater than a reference level of a SERPINE2 polypeptide. In some cases, a reference level of an mRNA encoding a SERPINE2 polypeptide in humans can be a level of such mRNA that is from about 0.4 pg/melanoma cell to about 5.47 pg/melanoma cell. For example, an increased level of mRNA encoding a SERPINE2 polypeptide can be a level that is at least 5% (e.g., about 5%, about 10%, about 20%, about 30%, about 40%, about 50%, about 60%, about 70%, about 80%, about 90%, about 95%, or more) higher than a reference level of mRNA encoding a SERPINE2 polypeptide. For example, an increased level of mRNA encoding a SERPINE2 polypeptide can be a level that is at least 2 (e.g., at least 5, at least 10, at least 15, at least 20, at least 25, at least 35, or at least 50) fold greater than a reference level of mRNA encoding a SERPINE2 polypeptide.

In some cases, an altered level of a polypeptide (or mRNA) can be an increased level of an ITGB3 polypeptide (or an increased level of an mRNA encoding an ITGB3 polypeptide). Examples of ITGB3 polypeptides and nucleic acid sequences encoding an ITGB3 polypeptide include, without limitation, those set forth in the NCBI databases at, for example, accession no. P05106 (version P05106.2). For example, an increased level of an ITGB3 polypeptide (or an mRNA encoding an ITGB3 polypeptide) can be any level that is higher than a reference level of the ITGB3 polypeptide (or a reference level of an mRNA encoding an ITGB3 polypeptide). In some cases, a reference level of an ITGB3 polypeptide in humans can be a level of an ITGB3 polypeptide in a sample (e.g., a tissue sample containing one or more melanoma cells) obtained from the human of from about 1.4 pg/melanoma cell to about 3.1 pg/melanoma cell. For example, an increased level of an ITGB3 polypeptide can be at least 5% (e.g., about 5%, about 10%, about 20%, about 30%, about 40%, about 50%, about 60%, about 70%, about 80%, about 90%, about 95%, or more) higher than a reference level of an ITGB3 polypeptide. For example, an increased level of an ITGB3 polypeptide can be a level that is at least 2 (e.g., at least 5, at least 10, at least 15, at least 20, at least 25, at least 35, or at least 50) fold greater than a reference level of an ITGB3 polypeptide. In some cases, a reference level of an mRNA encoding an ITGB3 polypeptide in humans can be a level of such mRNA that is from about 0.34 pg/melanoma cell to about 0.74 pg/melanoma cell. For example, an increased level of mRNA encoding an ITGB3 polypeptide can be a level that is at least 5% (e.g., about 5%, about 10%, about 20%, about 30%, about 40%, about 50%, about 60%, about 70%, about 80%, about 90%, about 95%, or more) higher than a reference level of mRNA encoding an ITGB3 polypeptide. For example, an increased level of mRNA encoding an ITGB3 polypeptide can be a level that is at least 2 (e.g., at least 5, at least 10, at least 15, at least 20, at least 25, at least 35, or at least 50) fold greater than a reference level of mRNA encoding an ITGB3 polypeptide.

In some cases, an altered level of a polypeptide (or mRNA) can be an increased level of an IGF1R polypeptide (or an increased level of an mRNA encoding an IGF1R polypeptide). Examples of IGF1R polypeptides and nucleic acid sequences encoding an IGF1R polypeptide include, without limitation, those set forth in the NCBI databases at, for example, accession no. AAI43722 (version AAI43722.1). For example, an increased level of an IGF1R polypeptide (or an mRNA encoding an IGF1R polypeptide) can be any level that is higher than a reference level of the IGF1R polypeptide (or a reference level of an mRNA encoding an IGF1R polypeptide). In some cases, a reference level of an IGF1R polypeptide in humans can be a level of an IGF1R polypeptide in a sample (e.g., a tissue sample containing one or more melanoma cells) obtained from the human of from about 1.4 pg/melanoma cell to about 3.1 pg/melanoma cell. For example, an increased level of an IGF1R polypeptide can be at least 5% (e.g., about 5%, about 10%, about 20%, about 30%, about 40%, about 50%, about 60%, about 70%, about 80%, about 90%, about 95%, or more) higher than a reference level of an IGF1R polypeptide. For example, an increased level of an IGF1R polypeptide can be a level that is at least 2 (e.g., at least 5, at least 10, at least 15, at least 20, at least 25, at least 35, or at least 50) fold greater than a reference level of an IGF1R polypeptide. In some cases, a reference level of an mRNA encoding an IGF1R polypeptide in humans can be a level of such mRNA that is from about 0.34 pg/melanoma cell to about 0.74 pg/melanoma cell. For example, an increased level of mRNA encoding an IGF1R polypeptide can be a level that is at least 5% (e.g., about 5%, about 10%, about 20%, about 30%, about 40%, about 50%, about 60%, about 70%, about 80%, about 90%, about 95%, or more) higher than a reference level of mRNA encoding an IGF1R polypeptide. For example, an increased level of mRNA encoding an IGF1R polypeptide can be a level that is at least 2 (e.g., at least 5, at least 10, at least 15, at least 20, at least 25, at least 35, or at least 50) fold greater than a reference level of mRNA encoding an IGF1R polypeptide.

In some cases, an altered level of a polypeptide (or mRNA) can be a decreased level of a CD200R1 polypeptide (or a decreased level of an mRNA encoding a CD200R1 polypeptide). Examples of CD200R1 polypeptides and nucleic acid sequences encoding a CD200R1 polypeptide include, without limitation, those set forth in the NCBI databases at, for example, accession no. AAI43394 (version AAI43394.1). For example, a decreased level of a CD200R1 polypeptide (or an mRNA encoding a CD200R1 polypeptide) can be any level that is lower than a reference level of the CD200R1 polypeptide (or a reference level of an mRNA encoding a CD200R1 polypeptide). In some cases, a reference level of a CD200R1 polypeptide in humans can be a level of a CD200R1 polypeptide in a sample (e.g., a tissue sample containing one or more melanoma cells) obtained from the human of from about 1.1 pg/melanoma cell to about 1.8 pg/melanoma cell. For example, a decreased level of a CD200R1 polypeptide can be at least 5% (e.g., about 5%, about 10%, about 20%, about 30%, about 40%, about 50%, about 60%, about 70%, about 80%, about 90%, about 95%, or more) lower than a reference level of a CD200R1 polypeptide. For example, a decreased level of a CD200R1 polypeptide can be a level that is at least 2 (e.g., at least 5, at least 10, at least 15, at least 20, at least 25, at least 35, or at least 50) fold less than a reference level of a CD200R1 polypeptide. In some cases, a reference level of an mRNA encoding a CD200R1 polypeptide in humans can be a level of such mRNA that is from about 0.36 pg/melanoma cell to about 0.61 pg/melanoma cell. For example, a decreased level of mRNA encoding a TMEM163 polypeptide can be a level that is at least 5% (e.g., about 5%, about 10%, about 20%, about 30%, about 40%, about 50%, about 60%, about 70%, about 80%, about 90%, about 95%, or more) lower than a reference level of mRNA encoding a CD200R1 polypeptide. For example, a decreased level of mRNA encoding a CD200R1 polypeptide can be a level that is at least 2 (e.g., at least 5, at least 10, at least 15, at least 20, at least 25, at least 35, or at least 50) fold less than a reference level of mRNA encoding a CD200R1 polypeptide.

In some cases, an altered level of a polypeptide (or mRNA) can be a decreased level of a SIRPG polypeptide (or a decreased level of an mRNA encoding a SIRPG polypeptide). Examples of SIRPG polypeptides and nucleic acid sequences encoding a SIRPG polypeptide include, without limitation, those set forth in the NCBI databases at, for example, accession no. Q9P1W8 (version Q9P1W8.3). For example, a decreased level of a SIRPG polypeptide (or an mRNA encoding a SIRPG polypeptide) can be any level that is lower than a reference level of the SIRPG polypeptide (or a reference level of an mRNA encoding a SIRPG polypeptide). In some cases, a reference level of a SIRPG polypeptide in humans can be a level of a SIRPG polypeptide in a sample (e.g., a tissue sample containing one or more melanoma cells) obtained from the human of from about 2.3 pg/melanoma cell to about 3.5 pg/melanoma cell. For example, a decreased level of a SIRPG polypeptide can be at least 5% (e.g., about 5%, about 10%, about 20%, about 30%, about 40%, about 50%, about 60%, about 70%, about 80%, about 90%, about 95%, or more) lower than a reference level of a SIRPG polypeptide. For example, a decreased level of a SIRPG polypeptide can be a level that is at least 2 (e.g., at least 5, at least 10, at least 15, at least 20, at least 25, at least 35, or at least 50) fold less than a reference level of a SIRPG polypeptide. In some cases, a reference level of an mRNA encoding a SIRPG polypeptide in humans can be a level of such mRNA that is from about 0.33 pg/melanoma cell to about 0.5 pg/melanoma cell. For example, a decreased level of mRNA encoding a SIRPG polypeptide can be a level that is at least 5% (e.g., about 5%, about 10%, about 20%, about 30%, about 40%, about 50%, about 60%, about 70%, about 80%, about 90%, about 95%, or more) lower than a reference level of mRNA encoding a SIRPG polypeptide. For example, a decreased level of mRNA encoding a SIRPG polypeptide can be a level that is at least 2 (e.g., at least 5, at least 10, at least 15, at least 20, at least 25, at least 35, or at least 50) fold less than a reference level of mRNA encoding a SIRPG polypeptide.

In some cases, an altered level of a polypeptide (or mRNA) can be a decreased level of a BTC polypeptide (or a decreased level of an mRNA encoding a BTC polypeptide). Examples of BTC polypeptides and nucleic acid sequences encoding a BTC polypeptide include, without limitation, those set forth in the NCBI databases at, for example, accession no. P35070 (version P35070.1). For example, a decreased level of a BTC polypeptide (or an mRNA encoding a BTC polypeptide) can be any level that is lower than a reference level of the BTC polypeptide (or a reference level of an mRNA encoding a BTC polypeptide). In some cases, a reference level of a BTC polypeptide in humans can be a level of a BTC polypeptide in a sample (e.g., a tissue sample containing one or more melanoma cells) obtained from the human of from about 0.5 pg/melanoma cell to about 1.4 pg/melanoma cell. For example, a decreased level of a BTC polypeptide can be at least 5% (e.g., about 5%, about 10%, about 20%, about 30%, about 40%, about 50%, about 60%, about 70%, about 80%, about 90%, about 95%, or more) lower than a reference level of a BTC polypeptide. For example, a decreased level of a BTC polypeptide can be a level that is at least 2 (e.g., at least 5, at least 10, at least 15, at least 20, at least 25, at least 35, or at least 50) fold less than a reference level of a BTC polypeptide. In some cases, a reference level of an mRNA encoding a BTC polypeptide in humans can be a level of such mRNA that is from about 0.24 pg/melanoma cell to about 0.65 pg/melanoma cell. For example, a decreased level of mRNA encoding a BTC polypeptide can be a level that is at least 5% (e.g., about 5%, about 10%, about 20%, about 30%, about 40%, about 50%, about 60%, about 70%, about 80%, about 90%, about 95%, or more) lower than a reference level of mRNA encoding a BTC polypeptide. For example, a decreased level of mRNA encoding a BTC polypeptide can be a level that is at least 2 (e.g., at least 5, at least 10, at least 15, at least 20, at least 25, at least 35, or at least 50) fold less than a reference level of mRNA encoding a BTC polypeptide.

In some cases, an altered level of a polypeptide (or mRNA) can be a decreased level of an OR52N4 polypeptide (or a decreased level of an mRNA encoding an OR52N4 polypeptide). Examples of OR52N4 polypeptides and nucleic acid sequences encoding an OR52N4 polypeptide include, without limitation, those set forth in the NCBI databases at, for example, accession no. Q8NGI2 (version Q8NGI2.2). For example, a decreased level of an OR52N4 polypeptide (or an mRNA encoding an OR52N4 polypeptide) can be any level that is lower than a reference level of the OR52N4 polypeptide (or a reference level of an mRNA encoding an OR52N4 polypeptide). In some cases, a reference level of an OR52N4 polypeptide in humans can be a level of an OR52N4 polypeptide in a sample (e.g., a tissue sample containing one or more melanoma cells) obtained from the human of from about 0.7 pg/melanoma cell to about 1.4 pg/melanoma cell. For example, a decreased level of an OR52N4 polypeptide can be at least 5% (e.g., about 5%, about 10%, about 20%, about 30%, about 40%, about 50%, about 60%, about 70%, about 80%, about 90%, about 95%, or more) lower than a reference level of an OR52N4 polypeptide. For example, a decreased level of an OR52N4 polypeptide can be a level that is at least 2 (e.g., at least 5, at least 10, at least 15, at least 20, at least 25, at least 35, or at least 50) fold less than a reference level of an OR52N4 polypeptide. In some cases, a reference level of an mRNA encoding an OR52N4 polypeptide in humans can be a level of such mRNA that is from about 0.24 pg/melanoma cell to about 0.65 pg/melanoma cell. For example, a decreased level of mRNA encoding an OR52N4 polypeptide can be a level that is at least 5% (e.g., about 5%, about 10%, about 20%, about 30%, about 40%, about 50%, about 60%, about 70%, about 80%, about 90%, about 95%, or more) lower than a reference level of mRNA encoding an OR52N4 polypeptide. For example, a decreased level of mRNA encoding an OR52N4 polypeptide can be a level that is at least 2 (e.g., at least 5, at least 10, at least 15, at least 20, at least 25, at least 35, or at least 50) fold less than a reference level of mRNA encoding an OR52N4 polypeptide.

In some cases, an altered level of a polypeptide (or mRNA) can be a decreased level of an ETV7 polypeptide (or a decreased level of an mRNA encoding an ETV7 polypeptide). Examples of ETV7 polypeptides and nucleic acid sequences encoding an ETV7 polypeptide include, without limitation, those set forth in the NCBI databases at, for example, accession no. Q9Y603 (version Q9Y603.1). For example, a decreased level of an ETV7 polypeptide (or an mRNA encoding an ETV7 polypeptide) can be any level that is lower than a reference level of the ETV7 polypeptide (or a reference level of an mRNA encoding an ETV7 polypeptide). In some cases, a reference level of an ETV7 polypeptide in humans can be a level of an ETV7 polypeptide in a sample (e.g., a tissue sample containing one or more melanoma cells) obtained from the human of from about 1.6 pg/melanoma cell to about 3.6 pg/melanoma cell. For example, a decreased level of an ETV7 polypeptide can be at least 5% (e.g., about 5%, about 10%, about 20%, about 30%, about 40%, about 50%, about 60%, about 70%, about 80%, about 90%, about 95%, or more) lower than a reference level of an ETV7 polypeptide. For example, a decreased level of an ETV7 polypeptide can be a level that is at least 2 (e.g., at least 5, at least 10, at least 15, at least 20, at least 25, at least 35, or at least 50) fold less than a reference level of an ETV7 polypeptide. In some cases, a reference level of an mRNA encoding an ETV7 polypeptide in humans can be a level of such mRNA that is from about 0.24 pg/melanoma cell to about 0.52 pg/melanoma cell. For example, a decreased level of mRNA encoding an ETV7 polypeptide can be a level that is at least 5% (e.g., about 5%, about 10%, about 20%, about 30%, about 40%, about 50%, about 60%, about 70%, about 80%, about 90%, about 95%, or more) lower than a reference level of mRNA encoding an ETV7 polypeptide. For example, a decreased level of mRNA encoding an ETV7 polypeptide can be a level that is at least 2 (e.g., at least 5, at least 10, at least 15, at least 20, at least 25, at least 35, or at least 50) fold less than a reference level of mRNA encoding an ETV7 polypeptide.

In some cases, an altered level of a polypeptide (or mRNA) can be a decreased level of a TOX polypeptide (or a decreased level of an mRNA encoding a TOX polypeptide). Examples of TOX polypeptides and nucleic acid sequences encoding a TOX polypeptide include, without limitation, those set forth in the NCBI databases at, for example, accession no. 094900 (version 094900.3). For example, a decreased level of a TOX polypeptide (or an mRNA encoding a TOX polypeptide) can be any level that is lower than a reference level of the TOX polypeptide (or a reference level of an mRNA encoding a TOX polypeptide). In some cases, a reference level of a TOX polypeptide in humans can be a level of a TOX polypeptide in a sample (e.g., a tissue sample containing one or more melanoma cells) obtained from the human of from about 1.6 pg/melanoma cell to about 3.1 pg/melanoma cell. For example, a decreased level of a TOX polypeptide can be at least 5% (e.g., about 5%, about 10%, about 20%, about 30%, about 40%, about 50%, about 60%, about 70%, about 80%, about 90%, about 95%, or more) lower than a reference level of a TOX polypeptide. For example, a decreased level of a TOX polypeptide can be a level that is at least 2 (e.g., at least 5, at least 10, at least 15, at least 20, at least 25, at least 35, or at least 50) fold less than a reference level of a TOX polypeptide. In some cases, a reference level of an mRNA encoding a TOX polypeptide in humans can be a level of such mRNA that is from about 0.41 pg/melanoma cell to about 0.76 pg/melanoma cell. For example, a decreased level of mRNA encoding a TOX polypeptide can be a level that is at least 5% (e.g., about 5%, about 10%, about 20%, about 30%, about 40%, about 50%, about 60%, about 70%, about 80%, about 90%, about 95%, or more) lower than a reference level of mRNA encoding a TOX polypeptide. For example, a decreased level of mRNA encoding a TOX polypeptide can be a level that is at least 2 (e.g., at least 5, at least 10, at least 15, at least 20, at least 25, at least 35, or at least 50) fold less than a reference level of mRNA encoding a TOX polypeptide.

In some cases, an altered level of a polypeptide (or mRNA) can be a decreased level of an ARSG polypeptide (or a decreased level of an mRNA encoding an ARSG polypeptide). Examples of ARSG polypeptides and nucleic acid sequences encoding an ARSG polypeptide include, without limitation, those set forth in the NCBI databases at, for example, accession no. Q96EG1 (version Q96EG1.1). For example, a decreased level of an ARSG polypeptide (or an mRNA encoding an ARSG polypeptide) can be any level that is lower than a reference level of the ARSG polypeptide (or a reference level of an mRNA encoding an ARSG polypeptide). In some cases, a reference level of an ARSG polypeptide in humans can be a level of an ARSG polypeptide in a sample (e.g., a tissue sample containing one or more melanoma cells) obtained from the human of from about 2.0 pg/melanoma cell to about 3.0 pg/melanoma cell. For example, a decreased level of an ARSG polypeptide can be at least 5% (e.g., about 5%, about 10%, about 20%, about 30%, about 40%, about 50%, about 60%, about 70%, about 80%, about 90%, about 95%, or more) lower than a reference level of an ARSG polypeptide. For example, a decreased level of an ARSG polypeptide can be a level that is at least 2 (e.g., at least 5, at least 10, at least 15, at least 20, at least 25, at least 35, or at least 50) fold less than a reference level of an ARSG polypeptide. In some cases, a reference level of an mRNA encoding an ARSG polypeptide in humans can be a level of such mRNA that is from about 0.57 pg/melanoma cell to about 0.84 pg/melanoma cell. For example, a decreased level of mRNA encoding an ARSG polypeptide can be a level that is at least 5% (e.g., about 5%, about 10%, about 20%, about 30%, about 40%, about 50%, about 60%, about 70%, about 80%, about 90%, about 95%, or more) lower than a reference level of mRNA encoding an ARSG polypeptide. For example, a decreased level of mRNA encoding an ARSG polypeptide can be a level that is at least 2 (e.g., at least 5, at least 10, at least 15, at least 20, at least 25, at least 35, or at least 50) fold less than a reference level of mRNA encoding an ARSG polypeptide.

In some cases, an altered level of a polypeptide (or mRNA) can be a decreased level of an ITGB7 polypeptide (or a decreased level of an mRNA encoding an ITGB7 polypeptide). Examples of ITGB7 polypeptides and nucleic acid sequences encoding an ITGB7 polypeptide include, without limitation, those set forth in the NCBI databases at, for example, accession no. P26010 (version P26010.1). For example, a decreased level of an ITGB7 polypeptide (or an mRNA encoding an ITGB7 polypeptide) can be any level that is lower than a reference level of the ITGB7 polypeptide (or a reference level of an mRNA encoding an ITGB7 polypeptide). In some cases, a reference level of an ITGB7 polypeptide in humans can be a level of an ITGB7 polypeptide in a sample (e.g., a tissue sample containing one or more melanoma cells) obtained from the human of from about 2.7 pg/melanoma cell to about 4.6 pg/melanoma cell. For example, a decreased level of an ITGB7 polypeptide can be at least 5% (e.g., about 5%, about 10%, about 20%, about 30%, about 40%, about 50%, about 60%, about 70%, about 80%, about 90%, about 95%, or more) lower than a reference level of an ITGB7 polypeptide. For example, a decreased level of an ITGB7 polypeptide can be a level that is at least 2 (e.g., at least 5, at least 10, at least 15, at least 20, at least 25, at least 35, or at least 50) fold less than a reference level of an ITGB7 polypeptide. In some cases, a reference level of an mRNA encoding an ITGB7 polypeptide in humans can be a level of such mRNA that is from about 0.3 pg/melanoma cell to about 0.51 pg/melanoma cell. For example, a decreased level of mRNA encoding an ITGB7 polypeptide can be a level that is at least 5% (e.g., about 5%, about 10%, about 20%, about 30%, about 40%, about 50%, about 60%, about 70%, about 80%, about 90%, about 95%, or more) lower than a reference level of mRNA encoding an ITGB7 polypeptide. For example, a decreased level of mRNA encoding an ITGB7 polypeptide can be a level that is at least 2 (e.g., at least 5, at least 10, at least 15, at least 20, at least 25, at least 35, or at least 50) fold less than a reference level of mRNA encoding an ITGB7 polypeptide.

In some cases, an altered level of a polypeptide (or mRNA) can be a decreased level of a CDKN2A polypeptide (or a decreased level of an mRNA encoding a CDKN2A polypeptide). Examples of CDKN2A polypeptides and nucleic acid sequences encoding a CDKN2A polypeptide include, without limitation, those set forth in the NCBI databases at, for example, accession no. P42771 (version P42771.2). For example, a decreased level of a CDKN2A polypeptide (or an mRNA encoding a CDKN2A polypeptide) can be any level that is lower than a reference level of the CDKN2A polypeptide (or a reference level of an mRNA encoding a CDKN2A polypeptide). In some cases, a reference level of a CDKN2A polypeptide in humans can be a level of a CDKN2A polypeptide in a sample (e.g., a tissue sample containing one or more melanoma cells) obtained from the human of from about 0.3 pg/melanoma cell to about 0.6 pg/melanoma cell. For example, a decreased level of a CDKN2A polypeptide can be at least 5% (e.g., about 5%, about 10%, about 20%, about 30%, about 40%, about 50%, about 60%, about 70%, about 80%, about 90%, about 95%, or more) lower than a reference level of a CDKN2A polypeptide. For example, a decreased level of a CDKN2A polypeptide can be a level that is at least 2 (e.g., at least 5, at least 10, at least 15, at least 20, at least 25, at least 35, or at least 50) fold less than a reference level of a CDKN2A polypeptide. In some cases, a reference level of an mRNA encoding a CDKN2A polypeptide in humans can be a level of such mRNA that is from about 0.07 pg/melanoma cell to about 0.17 pg/melanoma cell. For example, a decreased level of mRNA encoding a CDKN2A polypeptide can be a level that is at least 5% (e.g., about 5%, about 10%, about 20%, about 30%, about 40%, about 50%, about 60%, about 70%, about 80%, about 90%, about 95%, or more) lower than a reference level of mRNA encoding a CDKN2A polypeptide. For example, a decreased level of mRNA encoding a CDKN2A polypeptide can be a level that is at least 2 (e.g., at least 5, at least 10, at least 15, at least 20, at least 25, at least 35, or at least 50) fold less than a reference level of mRNA encoding a CDKN2A polypeptide.

In some cases, an altered level of a polypeptide (or mRNA) can be a decreased level of a DCD polypeptide (or a decreased level of an mRNA encoding a DCD polypeptide). Examples of DCD polypeptides and nucleic acid sequences encoding a DCD polypeptide include, without limitation, those set forth in the NCBI databases at, for example, accession no. P81605 (version P81605.2). For example, a decreased level of a DCD polypeptide (or an mRNA encoding a DCD polypeptide) can be any level that is lower than a reference level of the DCD polypeptide (or a reference level of an mRNA encoding a DCD polypeptide). In some cases, a reference level of a DCD polypeptide in humans can be a level of a DCD polypeptide in a sample (e.g., a tissue sample containing one or more melanoma cells) obtained from the human of from about 0.6 pg/melanoma cell to about 1703 pg/melanoma cell. For example, a decreased level of a DCD polypeptide can be at least 5% (e.g., about 5%, about 10%, about 20%, about 30%, about 40%, about 50%, about 60%, about 70%, about 80%, about 90%, about 95%, or more) lower than a reference level of a DCD polypeptide. For example, a decreased level of a DCD polypeptide can be a level that is at least 2 (e.g., at least 5, at least 10, at least 15, at least 20, at least 25, at least 35, or at least 50) fold less than a reference level of a DCD polypeptide. In some cases, a reference level of an mRNA encoding a DCD polypeptide in humans can be a level of such mRNA that is from about 0.1 pg/melanoma cell to about 271.94 pg/melanoma cell. For example, a decreased level of mRNA encoding a DCD polypeptide can be a level that is at least 5% (e.g., about 5%, about 10%, about 20%, about 30%, about 40%, about 50%, about 60%, about 70%, about 80%, about 90%, about 95%, or more) lower than a reference level of mRNA encoding a DCD polypeptide. For example, a decreased level of mRNA encoding a DCD polypeptide can be a level that is at least 2 (e.g., at least 5, at least 10, at least 15, at least 20, at least 25, at least 35, or at least 50) fold less than a reference level of mRNA encoding a DCD polypeptide.

Any appropriate method can be used to identify the presence, absence, or level of a polypeptide or mRNA encoding that polypeptide. In some cases, the presence, absence, or level of an mRNA encoding a polypeptide can be assessed by detecting and/or quantifying the mRNA encoding a polypeptide. Examples of methods that can be used to detect and/or quantify mRNA include, without limitation, RT-PCR techniques (e.g., quantitative RT-PCR techniques), next-generation RNA sequencing, mass spectrometry, RNA in situ hybridization, CRISPR-based methods, and nanopore-based methods. In some cases, the presence, absence, or level of a polypeptide can be assessed by detecting and/or quantifying the polypeptide. Examples of methods that can be used to detect and/or quantify polypeptides include, without limitation, immunohistochemistry (IHC) techniques, mass spectrometry techniques (e.g., proteomics-based mass spectrometry assays or targeted quantification-based mass spectrometry assays), Western blotting techniques, and enzyme-linked immunosorbent assays (ELISAs). In some cases, the presence, absence, or level of a polypeptide and/or mRNA encoding that polypeptide can be identified as described in Example 1. In some cases, the presence, absence, or level of a polypeptide and/or mRNA encoding that polypeptide can be identified as described in Example 2.

In some cases, a molecular signature used to determine whether or not a melanoma (e.g., a PCM) is likely to metastasize (e.g., likely to metastasize to one or more lymph nodes) can include one or more (e.g., one, two, or three) mutations in one or more (e.g., one, two, or three) polypeptides. For example, a sample (e.g., a tissue sample containing one or more melanoma cells) obtained from a mammal having melanoma (e.g., PCM) can be assessed for the presence or absence of one or more mutations in one or more polypeptides in a sample (e.g., a tissue sample containing one or more melanoma cells) obtained from the mammal to determine whether or not the melanoma is likely to metastasize (e.g., likely to metastasize to one or more lymph nodes). In some cases, a melanoma (e.g., PCM) can be identified as likely to metastasize (e.g., likely to metastasize to one or more lymph nodes) based, at least in part, on a molecular signature that includes the presence of one or more mutations in one or more polypeptides in a sample (e.g., a tissue sample containing one or more melanoma cells) obtained from a mammal having the melanoma. The term “mutation” as used herein with respect to a polypeptide refers to a modification in the amino acid sequence as compared to a wild type amino acid for a particular species. A mutation can be any type of mutation including, without limitation, an insertion of one or more amino acids, a deletion of one or more amino acids, a substitution of one or more amino acids, and combinations thereof. In some cases, a mutation in polypeptide can cause altered polypeptide activity.

A melanoma (e.g., PCM) that is likely to metastasize (e.g., likely to metastasize to one or more lymph nodes) can have the presence of one or more mutations in any appropriate polypeptides in a sample (e.g., a tissue sample containing one or more melanoma cells) obtained from a mammal having the melanoma. Examples of polypeptides that can be assessed to determine if one or more mutations are present or absent in the polypeptide within a sample (e.g., a tissue sample containing one or more melanoma cells) obtained from a mammal having melanoma (e.g., PCM) as described herein include, without limitation, a BRAF polypeptide and a NRAS polypeptide. In some cases, a polypeptide that can be assessed to determine if one or more mutations are present or absent in the polypeptide within a sample (e.g., a tissue sample containing one or more melanoma cells) obtained from a mammal having melanoma (e.g., PCM) can be as listed in Table 3.

In some cases, a mutation present in a molecular signature of a melanoma that is likely to metastasize (e.g., likely to metastasize to one or more lymph nodes) can be an amino acid substitution in a BRAF polypeptide. Examples of BRAF polypeptides and nucleic acid sequences encoding a BRAF polypeptide include, without limitation, those set forth in the NCBI databases at, for example, accession no. P15056 (version P15056.4) and accession no. NM_001374258 (version NM_001374258.1). In some cases, a mutation that can be present in a molecular signature that can be used to identify a mammal (e.g., a human) as having a melanoma (e.g., PCM) that is likely to metastasize (e.g., likely to metastasize to one or more lymph nodes) can be an amino acid substitution at residue 600 of a BRAF polypeptide (e.g., a BRAFV600 amino acid substitution). In some cases, a mutation that can be present in a molecular signature that can be used to identify a mammal (e.g., a human) as having a melanoma (e.g., PCM) that is likely to metastasize (e.g., likely to metastasize to one or more lymph nodes) can be as described elsewhere (see, e.g., Ascierto et al., J. Transl. Med., 10:85 (2012)). For example, the presence of a BRAFV600E substitution, a BRAFV600K substitution, a BRAFV600R substitution, or a BRAFV600D substitution in a molecular signature of a sample (e.g., a tissue sample containing one or more melanoma cells) obtained from a mammal (e.g., a human) having melanoma (e.g., PCM) can be used to identify the melanoma as being likely to metastasize to one or more lymph nodes within the mammal.

In some cases, a mutation present in a molecular signature of a melanoma that is likely to metastasize (e.g., likely to metastasize to one or more lymph nodes) can be an amino acid substitution in a NRAS polypeptide. Examples of NRAS polypeptides and nucleic acid sequences encoding a NRAS polypeptide include, without limitation, those set forth in the NCBI databases at, for example, accession no. P01111 (version P01111.1). In some cases, a mutation that can be present in a molecular signature that can be used to identify a mammal (e.g., a human) as having a melanoma (e.g., PCM) that is likely to metastasize (e.g., likely to metastasize to one or more lymph nodes) can be an amino acid substitution at residue 61 of a NRAS polypeptide (e.g., a NRASQ61 amino acid substitution). In some cases, a mutation that can be present in a molecular signature that can be used to identify a mammal (e.g., a human) as having a melanoma (e.g., PCM) that is likely to metastasize (e.g., likely to metastasize to one or more lymph nodes) can be as described elsewhere (see, e.g., Heppt et al., BMC Cancer, 17:536 (2017)). For example, the presence of a NRASQ61K substitution, a NRASQ61R substitution, a NRASQ61L substitution, a NRASQ61V substitution, or a NRASQ61H substitution in a molecular signature of a sample (e.g., a tissue sample containing one or more melanoma cells) obtained from a mammal (e.g., a human) having melanoma (e.g., PCM) can be used to identify the melanoma as being likely to metastasize to one or more lymph nodes within the mammal.

Any appropriate method can be used to detect the presence or absence of one or more mutations in a polypeptide within a sample (e.g., a sample containing one or more melanoma cells) obtained from a mammal (e.g., a human) having melanoma (e.g., PCM). For example, mass spectrometry, Edman degradation, and next-generation sequencing methods can be used to identify the presence or absence of one or more mutations in a polypeptide. In some cases, nucleic acid encoding a polypeptide (e.g., a BRAF polypeptide or a NRAS polypeptide) such as DNA or RNA can be assessed to detect the presence or absence of one or more mutations in that polypeptide within a sample (e.g., a sample containing one or more melanoma cells) obtained from a mammal (e.g., a human) having melanoma (e.g., PCM). For example, nucleic acid sequencing techniques can be used to detect the presence or absence of one or more mutations in a polypeptide (e.g., a BRAF polypeptide or a NRAS polypeptide).

Any appropriate sample from a mammal (e.g., a human) having melanoma (e.g., PCM) can be assessed as described herein (e.g., for the molecular signature of the melanoma). In some cases, a sample can be a biological sample. In some cases, a sample can contain one or more cancer cells (e.g., melanoma cells). In some cases, a sample can contain one or more biological molecules (e.g., nucleic acids such as DNA and RNA, polypeptides, carbohydrates, lipids, hormones, and/or metabolites). Examples of samples that can be assessed as described herein include, without limitation, tissue samples (e.g., skin tissue samples). A sample can be a fresh sample or a fixed sample (e.g., a formaldehyde-fixed sample or a formalin-fixed sample). In some cases, one or more biological molecules can be isolated from a sample (e.g., from one or more cancer cells within the sample). For example, nucleic acid can be isolated from a sample and can be assessed as described herein. For example, polypeptides can be isolated from a sample and can be assessed as described herein.

In some cases, methods and materials for determining whether or not a melanoma (e.g., a PCM) is likely to metastasize (e.g., likely to metastasize to one or more lymph nodes) as described herein (e.g., based, at least in part, on the molecular signature of the melanoma) also can include determining a Breslow depth of the melanoma. A Breslow depth of a melanoma can be from about 0.1 mm to about 10 mm. In some cases, a Breslow depth of greater than about 0.8 mm (e.g., from about 0.8 mm to about 10 mm, from about 0.8 mm to about 9 mm, from about 0.8 mm to about 8 mm, from about 0.8 mm to about 7 mm, from about 0.8 mm to about 6 mm, from about 0.8 mm to about 5 mm, from about 0.8 mm to about 4 mm, from about 0.8 mm to about 3 mm, from about 0.8 mm to about 2 mm, from about 0.8 mm to about 1 mm, from about 1 mm to about 10 mm, from about 2 mm to about 10 mm, from about 3 mm to about 10 mm, from about 4 mm to about 10 mm, from about 5 mm to about 10 mm, from about 6 mm to about 10 mm, from about 7 mm to about 10 mm, from about 8 mm to about 10 mm, from about 9 mm to about 10 mm, from about 1 mm to about 9 mm, from about 2 mm to about 8 mm, from about 3 mm to about 7 mm, from about 4 mm to about 6 mm, from about 1 mm to about 3 mm, from about 2 mm to about 4 mm, from about 3 mm to about 5 mm, from about 4 mm to about 6 mm, from about 5 mm to about 7 mm, from about 6 mm to about 8 mm, or from about 7 mm to about 9 mm) can indicate that a melanoma is likely to metastasize. For example, a Breslow depth of greater than about 0.8 mm in combination with the molecular signature of the melanoma can indicate that a melanoma is likely to metastasize. In some cases, a Breslow depth of less than about 0.8 mm (e.g., from about 0.1 mm to about 0.7 mm, from about 0.1 mm to about 0.6 mm, from about 0.1 mm to about 0.5 mm, from about 0.1 mm to about 0.4 mm, from about 0.1 mm to about 0.3 mm, from about 0.1 mm to about 0.2 mm, from about 0.2 mm to about 0.8 mm, from about 0.3 mm to about 0.8 mm, from about 0.4 mm to about 0.8 mm, from about 0.5 mm to about 0.8 mm, from about 0.6 mm to about 0.8 mm, from about 0.7 mm to about 0.8 mm, from about 0.2 mm to about 0.7 mm, from about 0.3 mm to about 0.6 mm, from about 0.4 mm to about 0.5 mm, from about 0.2 mm to about 0.4 mm, from about 0.3 mm to about 0.5 mm, from about 0.4 mm to about 0.6 mm, or from about 0.5 mm to about 0.7 mm) can indicate that a melanoma is not likely to metastasize. For example, a Breslow depth of less than about 0.8 mm in combination with the molecular signature of the melanoma can indicate that a melanoma is not likely to metastasize. Any appropriate method can be used to determine a Breslow depth of a melanoma (e.g., a PCM). For example, tumor depth of a skin sample (e.g., a skin sample obtained by excisional biopsy), an ocular micrometer at a right angle to the skin, and/or measurements obtained from digitized slides using computer software can be used to determine a Breslow depth of a melanoma (e.g., a PCM).

In some cases, a mammal (e.g., a human) having a melanoma (e.g., a PCM) that is identified as not being likely to metastasize (e.g., not likely to metastasize to one or more lymph nodes) as described herein (e.g., based, at least in part, on the molecular signature of the melanoma) can be selected to receive surgery to remove the melanoma. For example, a mammal having a melanoma (e.g., a PCM) that is identified as having a molecular signature that (a) lacks an increased level of a TMEM163 polypeptide (or an mRNA encoding a TMEM163 polypeptide), an increased level of a HEY1 polypeptide (or an mRNA encoding a HEY1 polypeptide), an increased level of an EYA4 polypeptide (or an mRNA encoding an EYA4 polypeptide), an increased level of a TMEM98 polypeptide (or an mRNA encoding a TMEM98 polypeptide), an increased level of a RLBP1 polypeptide (or an mRNA encoding a RLBP1 polypeptide), an increased level of an ELOVL2 polypeptide (or an mRNA encoding an ELOVL2 polypeptide), an increased level of a SERPINE2 polypeptide (or mRNA encoding a SERPINE2 polypeptide), an increased level of an ITGB3 polypeptide (or mRNA encoding an ITGB3 polypeptide), an increased level of an IGF1R polypeptide (or mRNA encoding an IGF1R polypeptide), a decreased level of a CD200R1 polypeptide (or an mRNA encoding a CD200R1 polypeptide), a decreased level of a SIRPG polypeptide (or an mRNA encoding a SIRPG polypeptide), a decreased level of a BTC polypeptide (or an mRNA encoding a BTC polypeptide), a decreased level of an OR52N4 polypeptide (or an mRNA encoding an OR52N4 polypeptide), a decreased level of an ETV7 polypeptide (or an mRNA encoding an ETV7 polypeptide), a decreased level of a TOX polypeptide (or an mRNA encoding a TOX polypeptide), a decreased level of an ARSG polypeptide (or an mRNA encoding an ARSG polypeptide), a decreased level of an ITGB7 polypeptide (or an mRNA encoding an ITGB7 polypeptide), a decreased level of a CDKN2A polypeptide (or mRNA encoding a CDKN2A polypeptide), and a decreased level of a DCD polypeptide (or mRNA encoding a DCD polypeptide); and (b) lacks a mutant BRAF polypeptide and a mutant NRAS polypeptide, can be selected to undergo surgery to remove the melanoma (e.g., in the absence of any adjuvant therapies).

In some cases, a mammal (e.g., a human) having a melanoma (e.g., a PCM) that is identified as being likely to metastasize (e.g., likely to metastasize to one or more lymph nodes) as described herein (e.g., based, at least in part, on the molecule signature of the melanoma) can be selected to receive surgery to remove the melanoma and can be selected for increased monitoring. For example, a mammal having a melanoma (e.g., a PCM) that is identified as having a molecular signature that includes (a) at least 11 of: an increased level of a TMEM163 polypeptide (or an mRNA encoding a TMEM163 polypeptide), an increased level of a HEY1 polypeptide (or an mRNA encoding a HEY1 polypeptide), an increased level of an EYA4 polypeptide (or an mRNA encoding an EYA4 polypeptide), an increased level of a TMEM98 polypeptide (or an mRNA encoding a TMEM98 polypeptide), an increased level of a RLBP1 polypeptide (or an mRNA encoding a RLBP1 polypeptide), an increased level of an ELOVL2 polypeptide (or an mRNA encoding an ELOVL2 polypeptide), an increased level of a SERPINE2 polypeptide (or mRNA encoding a SERPINE2 polypeptide), an increased level of an ITGB3 polypeptide (or mRNA encoding an ITGB3 polypeptide), an increased level of an IGF1R polypeptide (or mRNA encoding an IGF1R polypeptide), a decreased level of a CD200R1 polypeptide (or an mRNA encoding a CD200R1 polypeptide), a decreased level of a SIRPG polypeptide (or an mRNA encoding a SIRPG polypeptide), a decreased level of a BTC polypeptide (or an mRNA encoding a BTC polypeptide), a decreased level of an OR52N4 polypeptide (or an mRNA encoding an OR52N4 polypeptide), a decreased level of an ETV7 polypeptide (or an mRNA encoding an ETV7 polypeptide), a decreased level of a TOX polypeptide (or an mRNA encoding a TOX polypeptide), a decreased level of an ARSG polypeptide (or an mRNA encoding an ARSG polypeptide), a decreased level of an ITGB7 polypeptide (or an mRNA encoding an ITGB7 polypeptide), a decreased level of a CDKN2A polypeptide (or mRNA encoding a CDKN2A polypeptide), and/or a decreased level of a DCD polypeptide (or mRNA encoding a DCD polypeptide); and/or (b) one or more mutations in a BRAF polypeptide and/or a NRAS polypeptide can be selected to receive one or more (e.g., one, two, three, four, five, or more) tests that can be used to identify whether or not a melanoma (e.g., PCM) has metastasized.

In some cases, a mammal (e.g., a human) having a melanoma (e.g., a PCM) that is identified as being likely to metastasize (e.g., likely to metastasize to one or more lymph nodes) as described herein (e.g., based, at least in part, on the molecular signature of the melanoma) can be selected to receive surgery to remove the melanoma and can be selected for treatment with one or more adjuvant therapies. For example, a mammal having a melanoma (e.g., a PCM) that is identified as having a molecular signature that includes (a) at least 11 of: an increased level of a TMEM163 polypeptide (or an mRNA encoding a TMEM163 polypeptide), an increased level of a HEY1 polypeptide (or an mRNA encoding a HEY1 polypeptide), an increased level of an EYA4 polypeptide (or an mRNA encoding an EYA4 polypeptide), an increased level of a TMEM98 polypeptide (or an mRNA encoding a TMEM98 polypeptide), an increased level of a RLBP1 polypeptide (or an mRNA encoding a RLBP1 polypeptide), an increased level of an ELOVL2 polypeptide (or an mRNA encoding an ELOVL2 polypeptide), an increased level of a SERPINE2 polypeptide (or mRNA encoding a SERPINE2 polypeptide), an increased level of an ITGB3 polypeptide (or mRNA encoding an ITGB3 polypeptide), an increased level of an IGF1R polypeptide (or mRNA encoding an IGF1R polypeptide), a decreased level of a CD200R1 polypeptide (or an mRNA encoding a CD200R1 polypeptide), a decreased level of a SIRPG polypeptide (or an mRNA encoding a SIRPG polypeptide), a decreased level of a BTC polypeptide (or an mRNA encoding a BTC polypeptide), a decreased level of an OR52N4 polypeptide (or an mRNA encoding an OR52N4 polypeptide), a decreased level of an ETV7 polypeptide (or an mRNA encoding an ETV7 polypeptide), a decreased level of a TOX polypeptide (or an mRNA encoding a TOX polypeptide), a decreased level of an ARSG polypeptide (or an mRNA encoding an ARSG polypeptide), a decreased level of an ITGB7 polypeptide (or an mRNA encoding an ITGB7 polypeptide), a decreased level of a CDKN2A polypeptide (or mRNA encoding a CDKN2A polypeptide), and/or a decreased level of a DCD polypeptide (or mRNA encoding a DCD polypeptide); and/or (b) one or more mutations in a BRAF polypeptide and/or a NRAS polypeptide can be selected to undergo surgery to remove the melanoma and to receive one or more (e.g., one, two, three, four, five, or more) adjuvant therapies.

This document also provides methods for treating a mammal (e.g., a human) having melanoma (e.g., a PCM). In some cases, a mammal (e.g., a human) having a melanoma (e.g., a PCM) that is assessed as described herein (e.g., to determine whether or not the melanoma is likely to metastasize based, at least in part, on the molecular signature of the melanoma) can be administered or instructed to self-administer one or more (e.g., one, two, three, four, five, or more) cancer treatments, where the one or more cancer treatments are effective to treat the cancer within the mammal. For example, a mammal having melanoma (e.g., a PCM) can be administered or instructed to self-administer one or more cancer treatments selected based, at least in part, on whether or not the melanoma is likely to metastasize (e.g., based, at least in part, on the molecular signature of the melanoma).

In general, a cancer treatment for melanoma (e.g., a PCM) can include any appropriate melanoma cancer treatment. In some cases, a cancer treatment for melanoma (e.g., a PCM) can include surgery and other medical interventions. Examples of surgeries and other medical interventions that can be performed on a mammal having melanoma (e.g., a PCM) to treat the mammal include, without limitation, surgery (e.g., to remove a melanoma, to remove the tissue around a melanoma, and to remove a melanoma and the surrounding tissue), and radiation therapy. In some cases, a cancer treatment can include administering one or more anti-cancer drugs (e.g., chemotherapeutic agents, targeted cancer drugs, and immunotherapy drugs) to a mammal in need thereof. Examples of anti-cancer drugs that can be administered to a mammal having melanoma (e.g., a PCM) can include, without limitation, nivolumab (e.g., OPDIVO®), ipilimumab (e.g., YERVOY®), pembrolizumab (e.g., KEYTRUDA®), talimogene laherparepvec (T-VEC; e.g., IMLYGIC®), vemurafenib (e.g., ZELBORAF®), dabrafenib (e.g., TAFINLAR®), encorafenib (e.g., BRAFTOVI®), trametinib (e.g., MEKINIST®), cobimetinib (e.g., COTELLIC®), binimetinib (e.g., MEKTOVI®), atezolizumab (e.g., TECENTRIQ®), avelumab (e.g., BAVENCIO®), durvalumab (e.g., IMFINZI®), and combinations thereof.

When treating a mammal (e.g., a human) having a melanoma (e.g., a PCM) that is identified as not being likely to metastasize (e.g., not likely to metastasize to one or more lymph nodes) as described herein (e.g., based, at least in part, on the molecular signature of the melanoma), the mammal can undergo surgery (e.g., local reexcision surgery) to remove the melanoma. For example, a mammal having a melanoma (e.g., a PCM) that is identified as having a molecular signature that (a) lacks an increased level of a TMEM163 polypeptide (or an mRNA encoding a TMEM163 polypeptide), an increased level of a HEY1 polypeptide (or an mRNA encoding a HEY1 polypeptide), an increased level of an EYA4 polypeptide (or an mRNA encoding an EYA4 polypeptide), an increased level of a TMEM98 polypeptide (or an mRNA encoding a TMEM98 polypeptide), an increased level of a RLBP1 polypeptide (or an mRNA encoding a RLBP1 polypeptide), an increased level of an ELOVL2 polypeptide (or an mRNA encoding an ELOVL2 polypeptide), an increased level of a SERPINE2 polypeptide (or mRNA encoding a SERPINE2 polypeptide), an increased level of an ITGB3 polypeptide (or mRNA encoding an ITGB3 polypeptide), an increased level of an IGF1R polypeptide (or mRNA encoding an IGF1R polypeptide), a decreased level of a CD200R1 polypeptide (or an mRNA encoding a CD200R1 polypeptide), a decreased level of a SIRPG polypeptide (or an mRNA encoding a SIRPG polypeptide), a decreased level of a BTC polypeptide (or an mRNA encoding a BTC polypeptide), a decreased level of an OR52N4 polypeptide (or an mRNA encoding an OR52N4 polypeptide), a decreased level of an ETV7 polypeptide (or an mRNA encoding an ETV7 polypeptide), a decreased level of a TOX polypeptide (or an mRNA encoding a TOX polypeptide), a decreased level of an ARSG polypeptide (or an mRNA encoding an ARSG polypeptide), a decreased level of an ITGB7 polypeptide (or an mRNA encoding an ITGB7 polypeptide), a decreased level of a CDKN2A polypeptide (or mRNA encoding a CDKN2A polypeptide), and a decreased level of a DCD polypeptide (or mRNA encoding a DCD polypeptide); and (b) lacks a mutant BRAF polypeptide and a mutant NRAS polypeptide can undergo surgery to remove the melanoma.

In some cases, when treating a mammal (e.g., a human) having a melanoma (e.g., a PCM) that is identified as not being likely to metastasize (e.g., not likely to metastasize to one or more lymph nodes) as described herein (e.g., based, at least in part, on the molecular signature of the melanoma), the surgery can be the sole cancer treatment used to treat the mammal. For example, a mammal having a melanoma (e.g., a PCM) that is identified as having a molecular signature that (a) lacks an increased level of a TMEM163 polypeptide (or an mRNA encoding a TMEM163 polypeptide), an increased level of a HEY1 polypeptide (or an mRNA encoding a HEY1 polypeptide), an increased level of an EYA4 polypeptide (or an mRNA encoding an EYA4 polypeptide), an increased level of a TMEM98 polypeptide (or an mRNA encoding a TMEM98 polypeptide), an increased level of a RLBP1 polypeptide (or an mRNA encoding a RLBP1 polypeptide), an increased level of an ELOVL2 polypeptide (or an mRNA encoding an ELOVL2 polypeptide), an increased level of a SERPINE2 polypeptide (or mRNA encoding a SERPINE2 polypeptide), an increased level of an ITGB3 polypeptide (or mRNA encoding an ITGB3 polypeptide), an increased level of an IGF1R polypeptide (or mRNA encoding an IGF1R polypeptide), a decreased level of a CD200R1 polypeptide (or an mRNA encoding a CD200R1 polypeptide), a decreased level of a SIRPG polypeptide (or an mRNA encoding a SIRPG polypeptide), a decreased level of a BTC polypeptide (or an mRNA encoding a BTC polypeptide), a decreased level of an OR52N4 polypeptide (or an mRNA encoding an OR52N4 polypeptide), a decreased level of an ETV7 polypeptide (or an mRNA encoding an ETV7 polypeptide), a decreased level of a TOX polypeptide (or an mRNA encoding a TOX polypeptide), a decreased level of an ARSG polypeptide (or an mRNA encoding an ARSG polypeptide), a decreased level of an ITGB7 polypeptide (or an mRNA encoding a ITGB7 polypeptide), a decreased level of a CDKN2A polypeptide (or mRNA encoding a CDKN2A polypeptide), and a decreased level of a DCD polypeptide (or mRNA encoding a DCD polypeptide); and (b) lacks a mutant BRAF polypeptide and a mutant NRAS polypeptide can undergo local reexcision surgery to remove the melanoma and is not administered or instructed to self-administer any additional (e.g., adjuvant) cancer treatments.

When treating a mammal (e.g., a human) having a melanoma (e.g., a PCM) that is identified as being likely to metastasize (e.g., likely to metastasize to one or more lymph nodes) as described herein (e.g., based, at least in part, on the molecular signature of the melanoma), the mammal can undergo surgery to remove the melanoma and can be administered or instructed to self-administer one or more adjuvant therapies. For example, a mammal having a melanoma (e.g., a PCM) that is identified as having a molecular signature that includes (a) at least 11 of: an increased level of a TMEM163 polypeptide (or an mRNA encoding a TMEM163 polypeptide), an increased level of a HEY1 polypeptide (or an mRNA encoding a HEY1 polypeptide), an increased level of an EYA4 polypeptide (or an mRNA encoding an EYA4 polypeptide), an increased level of a TMEM98 polypeptide (or an mRNA encoding a TMEM98 polypeptide), an increased level of a RLBP1 polypeptide (or an mRNA encoding a RLBP1 polypeptide), an increased level of an ELOVL2 polypeptide (or an mRNA encoding an ELOVL2 polypeptide), an increased level of a SERPINE2 polypeptide (or mRNA encoding a SERPINE2 polypeptide), an increased level of an ITGB3 polypeptide (or mRNA encoding an ITGB3 polypeptide), an increased level of an IGF1R polypeptide (or mRNA encoding an IGF1R polypeptide), a decreased level of a CD200R1 polypeptide (or an mRNA encoding a CD200R1 polypeptide), a decreased level of a SIRPG polypeptide (or an mRNA encoding a SIRPG polypeptide), a decreased level of a BTC polypeptide (or an mRNA encoding a BTC polypeptide), a decreased level of an OR52N4 polypeptide (or an mRNA encoding an OR52N4 polypeptide), a decreased level of an ETV7 polypeptide (or an mRNA encoding an ETV7 polypeptide), a decreased level of a TOX polypeptide (or an mRNA encoding a TOX polypeptide), a decreased level of an ARSG polypeptide (or an mRNA encoding an ARSG polypeptide), a decreased level of an ITGB7 polypeptide (or an mRNA encoding an ITGB7 polypeptide), a decreased level of a CDKN2A polypeptide (or mRNA encoding a CDKN2A polypeptide), and/or a decreased level of a DCD polypeptide (or mRNA encoding a DCD polypeptide); and/or (b) one or more mutations in a BRAF polypeptide and/or a NRAS polypeptide can undergo surgery to remove the melanoma and can be administered or instructed to self-administer one or more (e.g., one, two, three, four, five, or more) adjuvant therapies. An adjuvant therapy can be any appropriate cancer treatment. In some cases, a cancer treatment that can be used as an adjuvant therapy with surgery to treat a mammal having a melanoma (e.g., a PCM) that is identified as being likely to metastasize as described herein can include, without limitation, radiation therapy. In some cases, a cancer treatment that can be used as an adjuvant therapy with surgery to treat a mammal having a melanoma (e.g., a PCM) that is identified as being likely to metastasize as described herein can include administering one or more anti-cancer drugs (e.g., chemotherapeutic agents, targeted cancer drugs, and immunotherapy drugs) to the mammal. Examples of anti-cancer drugs that can be administered to a mammal having melanoma (e.g., a PCM) can include, without limitation, nivolumab (e.g., OPDIVO®), ipilimumab (e.g., YERVOY®), pembrolizumab (e.g., KEYTRUDA®), talimogene laherparepvec (T-VEC; e.g., IMLYGIC®), vemurafenib (e.g., ZELBORAF®), dabrafenib (e.g., TAFINLAR®), encorafenib (e.g., BRAFTOVI®), trametinib (e.g., MEKINIST®), cobimetinib (e.g., COTELLIC®), binimetinib (e.g., MEKTOVI®), atezolizumab (e.g., TECENTRIQ®), avelumab (e.g., BAVENCIO®), durvalumab (e.g., IMFINZI®), and combinations thereof.

In some cases, when treating a mammal (e.g., a human) having melanoma (e.g., PCM) as described herein, the treatment can be effective in treating the melanoma. For example, the number of cancer cells present within a mammal can be reduced using the methods and materials described herein. In some cases, the methods and materials described herein can be used to reduce the number of cancer cells present within a mammal having melanoma by, for example, 10, 20, 30, 40, 50, 60, 70, 80, 90, 95, or more percent. In some cases, the number of cancer cells present within a mammal does not increase. For example, the size (e.g., volume) of one or more tumors present within a mammal can be reduced using the methods and materials described herein. In some cases, the methods and materials described herein can be used to reduce the size of one or more tumors present within a mammal having melanoma (e.g., PCM) by, for example, 10, 20, 30, 40, 50, 60, 70, 80, 90, 95, or more percent. In some cases, the size (e.g., volume) of one or more tumors present within a mammal does not increase.

In some cases, when treating a mammal (e.g., a human) having melanoma (e.g., PCM) as described herein, the treatment can be effective to treat the melanoma while minimizing the risk of metastasis. For example, the number of cancer cells present within a mammal can be reduced using the methods and materials described herein. In some cases, the methods and materials described herein can be used to reduce the number of cancer cells present within a mammal having melanoma by, for example, 10, 20, 30, 40, 50, 60, 70, 80, 90, 95, or more percent while minimizing the risk of metastasis. In some cases, the number of cancer cells present within a mammal does not increase. For example, the size (e.g., volume) of one or more tumors present within a mammal can be reduced using the methods and materials described herein. In some cases, the methods and materials described herein can be used to reduce the size of one or more tumors present within a mammal having melanoma (e.g., PCM) by, for example, 10, 20, 30, 40, 50, 60, 70, 80, 90, 95, or more percent while minimizing the risk of metastasis. In some cases, the size (e.g., volume) of one or more tumors present within a mammal does not increase.

In some cases, when treating a mammal (e.g., a human) having melanoma (e.g., PCM) as described herein, the treatment can be effective to improve survival of the mammal. For example, the methods and materials described herein can be used to improve disease-free survival (e.g., metastasis-free survival). For example, the methods and materials described herein can be used to improve overall survival. For example, the methods and materials described herein can be used to improve the survival of a mammal having melanoma (e.g., PCM) by, for example, 10, 20, 30, 40, 50, 60, 70, 80, 90, 95, or more percent. For example, the methods and materials described herein can be used to improve the survival of a mammal having melanoma (e.g., PCM) by, for example, at least 6 months (e.g., about 6 months, about 8 months, about 10 months, about 1 year, about 1.5 years, about 2 years, about 2.5 years, about 3 years, about 3.5 years, about 4 years, about 4.5 years, or about 5 years).

In some cases, when treating a mammal (e.g., a human) having melanoma (e.g., PCM) as described herein, the treatment can be effective to improve survival of the mammal while minimizing the risk of metastasis. For example, the methods and materials described herein can be used to improve disease-free survival (e.g., metastasis-free survival). For example, the methods and materials described herein can be used to improve overall survival. For example, the methods and materials described herein can be used to improve the survival of a mammal having melanoma (e.g., PCM) by, for example, 10, 20, 30, 40, 50, 60, 70, 80, 90, 95, or more percent while minimizing the risk of metastasis. For example, the methods and materials described herein can be used to improve the survival of a mammal having melanoma (e.g., PCM) by, for example, at least 6 months (e.g., about 6 months, about 8 months, about 10 months, about 1 year, about 1.5 years, about 2 years, about 2.5 years, about 3 years, about 3.5 years, about 4 years, about 4.5 years, or about 5 years) while minimizing the risk of metastasis.

In some cases, when treating a mammal (e.g., a human) having melanoma (e.g., PCM) as described herein, the treatment can be effective in reducing or eliminating one or more symptoms of the melanoma. Examples of symptoms of melanoma (e.g., PCM) that can be reduced or eliminated using the methods and materials described herein can include, without limitation, local relapse of a melanoma moles, development of a new pigmented and/or unusual-looking growth on the skin (e.g., satellite metastases and in-transit metastases), sores and/or wounds that don't heal, redness and/or swelling that spreads outside the border of a spot to the surrounding skin, itchiness, tenderness, and pain. For example, the methods and materials described herein can be used to reduce one or more symptoms of melanoma (e.g., PCM) within a mammal having melanoma by, for example, 10, 20, 30, 40, 50, 60, 70, 80, 90, 95, or more percent.

In some cases, when treating a mammal (e.g., a human) having melanoma (e.g., PCM) as described herein, the treatment can be effective to reduce or eliminate one or more symptoms of the melanoma while minimizing the risk of metastasis. Examples of symptoms of melanoma (e.g., PCM) that can be reduced or eliminated using the methods and materials described herein can include, without limitation, local relapse of a melanoma moles, development of a new pigmented and/or unusual-looking growth on the skin (e.g., satellite metastases and in-transit metastases), sores and/or wounds that don't heal, redness and/or swelling that spreads outside the border of a spot to the surrounding skin, itchiness, tenderness, and pain. For example, the methods and materials described herein can be used to reduce one or more symptoms of melanoma (e.g., PCM) within a mammal having melanoma by, for example, 10, 20, 30, 40, 50, 60, 70, 80, 90, 95, or more percent while minimizing the risk of metastasis.

In some cases, when treating a mammal (e.g., a human) having melanoma (e.g., PCM) as described herein, the treatment can be effective to reduce or eliminate one or more complications associated with melanoma. A complication associated with melanoma can be a short-term complication or a long-term complication. Examples of complications associated with melanoma (e.g., PCM) that can be reduced or eliminated using the methods and materials described herein can include, without limitation, bleeding, infection, seroma, lymphocele, wound dehiscence, hematoma, lymphatic fistula, pain, neuropathy, lymphedema, and postsurgical complications. For example, the methods and materials described herein can be used to reduce one or more complications associated with melanoma (e.g., PCM) within a mammal having melanoma by, for example, 10, 20, 30, 40, 50, 60, 70, 80, 90, 95, or more percent.

In some cases, when treating a mammal (e.g., a human) having melanoma (e.g., PCM) as described herein, the treatment can be effective to reduce or eliminate one or more complications associated with melanoma while minimizing the risk of metastasis. Examples of symptoms of melanoma (e.g., PCM) that can be reduced or eliminated using the methods and materials described herein can include, without limitation, bleeding, infection, seroma, lymphocele, wound dehiscence, hematoma, lymphatic fistula, pain, neuropathy, lymphedema, and postsurgical complications. For example, the methods and materials described herein can be used to reduce one or more complications associated with melanoma (e.g., PCM) within a mammal having melanoma by, for example, 10, 20, 30, 40, 50, 60, 70, 80, 90, 95, or more percent while minimizing the risk of metastasis.

In some cases, a course of treatment, the number of cancer cells present within a mammal, the severity of one or more symptoms related to the melanoma (e.g., PCM), and/or the severity of one or more complications associated with melanoma can be monitored. Any appropriate method can be used to determine whether or not the number of cancer cells present within a mammal is reduced. For example, imaging techniques can be used to assess the number of cancer cells present within a mammal.

The disclosure will be further described in the following examples, which do not limit the scope of the disclosure described in the claims.

### EXAMPLES

### Example 1: a Prognostic Gene Expression Signature for Primary Cutaneous Melanoma

While the melanoma biomarker landscape is evolving, there are currently no generally accepted, thoroughly studied, guideline-recommended predictive or prognostic biomarkers available that identify early-stage high-risk disease (Grossman et al., Melanoma Management, 6:MMT32 (2019); and Grossman et al., JAMA Dermatol., 156:1004-11 (2020)).

This Example describes the identification of a gene expression signature for primary cutaneous melanoma that can be used to predict whether or not a melanoma patient is at a higher risk of metastasis (e.g., nodal metastasis).

### Discovery Cohort

For the discovery of the classifier, a subset of an 837 patient, prospectively-designed archival discovery cohort was used. This cohort has been characterized as described elsewhere (Eggermont et al., Eur. J. Cancer, 140:11-8 (2020); Bellomo et al., JCO Precision Oncology, 2020:319-34 (2020); Meves et al., J. Clin. Oncol., 33:2509-15 (2015); Kung et al., J. Invest. Dermatol., 140:2301-4.e3 (2020); Quattrocchi et al., Int. J. Dermatol., 59:1241-8 (2020); and Meves et al., Mayo Clin. Proc. Innov. Qual. Outcomes, 4:586-7 (2020)). Briefly, this is a cohort of melanoma patients who had a SLNb performed within 90 days of their diagnosis (a time interval shown not to affect SLNb positivity or survival rates). Electronic searches of pathology reports retrospectively identified patients with primary cutaneous melanoma with known SLNb status.

### Eligibility

Eligibility was based on histopathology data derived from patient medical records and established by two or more board-certified dermatopathologists. Inclusion was determined by institutional practice guidelines for recommending SLNb, which were based on Breslow thickness, ulceration, mitoses, and patient age. Patients were eligible for this study if they met one of the following three conditions: 1) Breslow thickness greater than 1.0 mm; 2) Breslow thickness of 0.75-0.99 mm and presence of ulceration, mitoses, or patient age less than 40 years; or 3) Breslow thickness of 0.50-0.74 mm and presence of at least two of the following: ulceration, mitoses and patient age less than 40 years.

### Exclusion Criteria

Exclusion criteria were as follows: M1 disease within 90 days of primary diagnosis; insufficient primary tumor diagnostic biopsy tissue; inadequate RNA harvested and, denial of access to medical records for research purposes. Patients were not excluded based on histologic type, but histologic type was recorded as a clinicopathologic variable.

### Patient and Tumor Characteristics

Patient and tumor characteristics are as described elsewhere (Eggermont et al., Eur. J. Cancer, 140:11-8 (2020)). Most patients, namely 580/837 or 69%, were diagnosed with early-stage I/IIA disease. 50/837 patients or 6% developed brain metastases. 78/837 or 9% of patients presented with stage IIIA disease, 42 of these 78 patients had low-volume SLNb metastasis (individual tumor cells or cell clusters <0.1 mm).

### RNA Sequencing

RNA sequencing data, which was used for the discovery of the disclosed CP-GEP classifier, was derived from a subset of 382 of the 837 patients:
384 samples were selected from the 837-patient cohort based on RNA quality, i.e., RIN scores, DV200 scores, concentration, fragment sizes, and the presence of primer dimers.
Relapse-free survival (RFS): The majority of patients (52%) in the RNAseq group were diagnosed with stage I/IIA disease (199/384). Median follow-up was 43.7 months; 87/384 (23%) of patients experienced a relapse as of Dec. 1, 2019.
RNA libraries were prepared according to the manufacturer's instructions for the TRUSEQ® RNA Exome Library Prep Kit by Illumina (San Diego, CA).
Coding regions of the transcriptome were captured by pooling four of the cDNA libraries at 200 ng each, following the TRUSEQ® RNA Exome Library Prep Kit instructions.
Libraries were multiplexed at 96 samples per flow cell, following Illumina's standard protocol for the Illumina NovaSeq™ 6000 and S4 flow cell. The flow cells were sequenced as 100×2 paired-end reads using the NovaSeq™ S4 sequencing kit and NovaSeq™ Control Software v1.6.0. Base-calling was performed using Illumina's RTA version 3.4.4.
An S4 flow cell's total output is nine billion single-end reads, or 18 billion paired-end reads passing filters (about 94 million single-end reads per sample).

### RNA Sequencing Data—Quality Control

Quality Control (QC) was performed using MultiQC.
Total samples sequenced: 384.
Sample MM725 failed sequencing.
The remaining 383 samples showed the following characteristics:
   Average sequencing depth (million reads): 91 [range: 13.5-502].
   Average mapped reads (%): 81 [range: 71-87]; this means that on average, ˜19% of reads were unmapped, which falls within the normal, expected range.
   Average ribosomal reads (%): 0.57 [range: 0.1-1.8]; this is excellent; no reads were lost to ribosomal regions.
Included into downstream analysis: 383 of 384 total samples.
Overall, the QC of the samples looked excellent.

### Example 2: CP-GEP Models for Predicting sLNB-Positivity

A LASSO double-loop cross-validation (DLCV), a logistic regression approach, was used to build a model to predict SLNb status as described elsewhere (Bellomo et al., JCO Precision Oncology, 2020:319-34 (2020)). Briefly, the following steps were followed:
(1) the study set was randomly sorted.
(2) 10-fold cross-validation was used to estimate the LASSO penalty term.
(3) 3-fold cross-validation and the penalty term from (2) were used to build/train a predictive model on two-thirds of the data. The remaining one-third was used to test the model on, resulting in 3 predictive models. This process is repeated 100 times, generating a total of 300 predictive models.
(4) one final model was created by fitting all the variables with the LASSO logistic regression using a penalty term equal to the average of the penalty terms from the 100 loops.

Due to the way the 3-fold cross-validation approach assigns subjects to training and testing sets, each subject is included in exactly 100 of the 300 model test sets. Final probability estimates of SLN finding for a given subject were then calculated by averaging their estimates across the 100 test set models. The overall predictive capability of the model is assessed using receiver operating characteristics (ROC curves).

The variables most predictive of finding a SLN biopsy were determined based on the proportion of times they were included in the 300 LASSO models.

### Gene Filtering Approach.

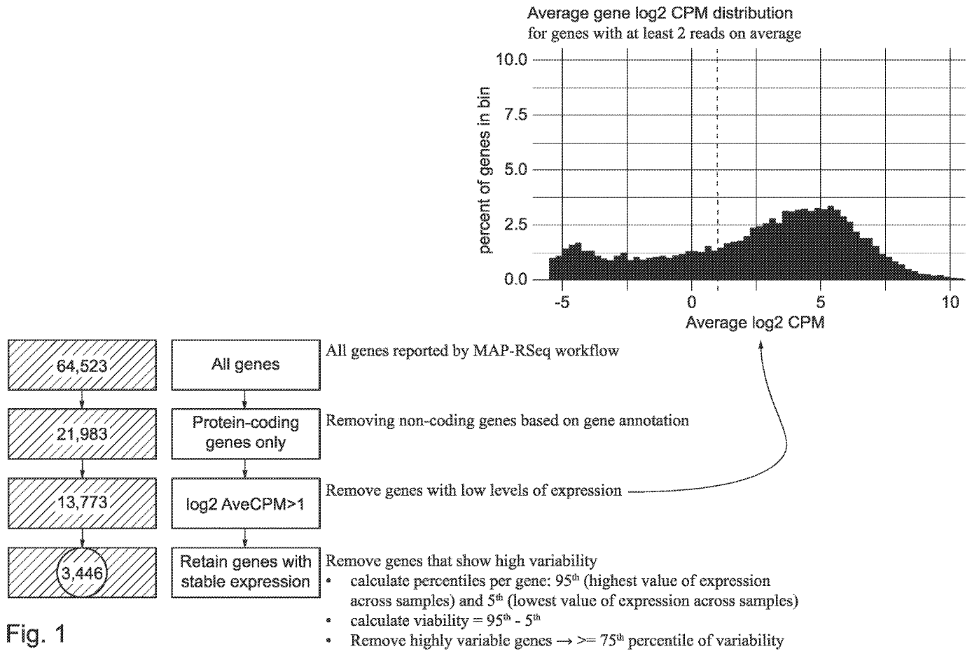

All genes reported by the MAP-RSeq workflow (N=64,523) were filtered as follows:
noncoding genes were removed.
genes with low expression were removed.
genes with low variability in expression across samples were removed.
See FIG. 1 for details.

### Gene Lists Used for Modeling

Approach A: a gene list, which includes only protein-coding genes, was provided; low expressing genes were eliminated (based on average log FPKM values); genes were not excluded based on low variability; number of predictor variables=13,777.
Approach B: Approach A+genes excluded based on low variability; number of predictor variables=3,450.

### CP-GEP Models Predicting Relapse

Approach A: a total of 596 genes were selected at least once into the 300 models generated by DLCV.
The final LASSO model was generated by fitting all the variables with Cox LASSO regression using a penalty term equal to the average of the penalty terms from the 100 loops.
The final model contained Breslow depth as a clinicopathologic variable as well as 18 genes (see Table 1).

TABLE 1
List from Approach A

| Variable Type | Variable | ENSMBL |
|---|---|---|
| CP variable | Breslow depth | — |
| GEP | TMEM98 | ENSG00000006042 |
| GEP | HDAC9 | ENSG00000048052 |
| GEP | NFX1 | ENSG00000086102 |
| GEP | EYA4 | ENSG00000112319 |
| GEP | FCHO1 | ENSG00000130475 |
| GEP | CYP2E1 | ENSG00000130649 |
| GEP | MYH10 | ENSG00000133026 |
| GEP | MTO1 | ENSG00000135297 |
| GEP | LEF1 | ENSG00000138795 |
| GEP | RLBP1 | ENSG00000140522 |
| GEP | TMEM163 | ENSG00000152128 |
| GEP | HEY1 | ENSG00000164683 |
| GEP | CTHRC1 | ENSG00000164932 |
| GEP | OTUD1 | ENSG00000165312 |
| GEP | FAM175B | ENSG00000165660 |
| GEP | GJB1 | ENSG00000169562 |
| GEP | XCR1 | ENSG00000173578 |
| GEP | FAM53B | ENSG00000189319 |

Approach B: a total of 353 genes were selected at least once into the 300 models generated by DLCV.
The final LASSO model was generated by fitting all the variables with Cox LASSO regression using a penalty term equal to the average of the penalty terms from the 100 loops.
The final model contained Breslow depth as a clinicopathologic variable as well as 6 genes (see Table 2).

TABLE 2
List from Approach B

| Variable Type | Variable | ENSMBL |
|---|---|---|
| CP variable | Breslow depth | — |
| GEP | TMEM98 | ENSG00000006042 |
| GEP | ETV7 | ENSG00000010030 |
| GEP | SIRPG | ENSG00000089012 |
| GEP | EYA4 | ENSG00000112319 |
| GEP | ITGB7 | ENSG00000139626 |
| GEP | RLBP1 | ENSG00000140522 |
| GEP | ARSG | ENSG00000141337 |
| GEP | TMEM163 | ENSG00000152128 |
| GEP | CD200R1 | ENSG00000163606 |
| GEP | HEY1 | ENSG00000164683 |
| GEP | BTC | ENSG00000174808 |
| GEP | OR52N4 | ENSG00000181074 |
| GEP | ELOVL2 | ENSG00000197977 |
| GEP | TOX | ENSG00000198846 |

### Performance: Relapse-Free Survival (Binarized); Area Under the Curve (AUC)

| | CP variables | Approach A | Approach A | Approach B | Approach B |
|---|---|---|---|---|---|
| | only (status | (GEP) - genes | (CP-GEP) - | (GEP) - genes | (CP-GEP) - |
| | quo) | only | clinical + genes | only | clinical + genes |
| AUC | 0.74 | 0.76 | 0.77 | 0.77 | 0.765 |

Gene expression variables add value to predicting relapse-free survival
Both CP-GEP performed similarly
CP-GEP in Approach B—despite using fewer genes—appears to work as well as Approach A CP-GEP

Genes demonstrating differential expression that can be used to predict melanoma metastasis are shown in FIG. 4.

### Mutation Data

The following mutations were examiner for predicting SLNb:
BRAFV600 mutations
NRASQ61 mutations

Univariately, the BRAFV600E mutation was strongly associated with SLNb metastasis, whereas NRASQ61 mutation was not.

| | WT | BRAFV600E | |
|---|---|---|---|
| SLNb(1) - with | 73 | 40 | 113 |
| metastasis | | | |
| SLNb(0) - no | 231 | 39 | 270 |
| metastasis | | | |
| | 304 | 79 | 383 |

χ2 test: p < 0.0001

Models incorporating BRAFV600E mutation data were also built. NRASQ61 was offered to the LASSO machine learning algorithm (binarized: 0=wt; 1=mutation present). The performance of the Final Model shown for Approach B was compared to the Final Model shown in Approach B plus BRAFV600E/NRAS Q61X mutation data. Results showed that the inclusion of mutation data improved the performance of the LASSO model:

| | Approach B: | Approach C (CP-GEP) - |
|---|---|---|
| | (CP-GEP) - | clinical + genes + |
| | clinical + genes | BRAF/NRAS mutation data |
| AUC | 0.796 | 0.804 |

As one can see, the final model C is updated from the final model B and contains the following clinical+ genes+mutation variables (Table 3):

TABLE 3
Variables for predicting melanoma sLNB metastasis

| Variable Type | Variable | ENSMBL |
|---|---|---|
| CP variable | Breslow depth | — |
| GEP | ARSG | ENSG00000141337 |
| GEP | EYA4 | ENSG00000112319 |
| GEP | TMEM163 | ENSG00000152128 |
| CP variable | Patient age | — |
| GEP | TMEM98 | ENSG00000006042 |
| Mutation | BRAFV600E | — |
| GEP | SIRPG | ENSG00000089012 |
| GEP | ELOVL2 | ENSG00000197977 |
| GEP | HEY1 | ENSG00000164683 |
| GEP | RLBP1 | ENSG00000140522 |
| Mutation | NRASQ61X | — |
| GEP | ETV7 | ENSG00000010030 |
| GEP | BTC | ENSG00000174808 |
| GEP | CD200R1 | ENSG00000163606 |

Together, these results demonstrate that an increased level of a TMEM163 polypeptide, an increased level of a HEY1 polypeptide, an increased level of an EYA4 polypeptide, an increased level of a TMEM98 polypeptide, an increased level of a RLBP1 polypeptide, an increased level of an ELOVL2 polypeptide, a decreased level of a CD200R1 polypeptide, a decreased level of a SIRPG polypeptide, a decreased level of a BTC polypeptide, a decreased level of an OR52N4 polypeptide, a decreased level of an ETV7 polypeptide, a decreased level of a TOX polypeptide, a decreased level of an ARSG polypeptide, a decreased level of an ITGB7 polypeptide, a mutation in a BRAF polypeptide, and a mutation in a NRAS polypeptide in melanoma cells of a mammal having melanoma can be used to identify that mammal as being likely to metastasize.

### Example 3: a Prognostic Gene Expression Signature for Primary Cutaneous Melanoma

### Methods

### Patient Cohort

The study cohort was derived from a previously published cohort of 854 patients diagnosed with stage I-III malignant melanoma who had an SLNb performed within 90 days of their diagnosis (Quattrocchi et al., Int. J. Dermatol., 59:1241-8 (2020)). Briefly, patients with primary cutaneous melanoma with known SLN status were retrospectively identified by electronic searches of pathology reports. Charts were then reviewed for eligibility criteria (see next paragraph), and if met, diagnostic biopsy tissue was requested. Patients underwent SLNb. Patients who presented with stage IV disease or who developed stage IV disease within 90 days of melanoma diagnosis were excluded from the cohort.

For the purpose of this analysis (quantitative PCR data analysis), patients with stage T3b (N=79) and T4 disease (N=31) at diagnosis (patients with advanced primary tumors) were excluded. Because there is an ongoing debate about the relevance of <0.1 mm metastasis in SLN (i.e., isolated tumor cells (ITCs) and cell clusters <0.1 mm in diameter), patients with <0.1 mm metastasis were excluded from model development (N=37).

All specimens were analyzed by quantitative polymerase chain reaction (qPCR). 26 additional patients were excluded for poor qPCR amplification. Thus, a total of 681 patients were included in the analysis.

### Quantitative Polymerase Chain Reaction

Quantitative polymerase chain reaction (PCR) was performed as previously described (Bellomo et al., JCO Precision Oncology, 2020:319-34 (2020)). Briefly, RNA purification was from formalin-fixed paraffin-embedded tissue (QIAGEN, Hilden, Germany). Quantitative reverse transcription PCR was done using the BioMark HD System and dynamic array integrated fluid circuits (Fluidigm, South San Francisco, CA). All cDNA was pre-amplified (TaqMan PreAmp Master Mix, Applied Biosystems, Foster City, CA). Array-based quantitative PCR was developed with the help of the TaqMan Gene Expression Master Mix (Applied Biosystems). After thermal cycling, raw Ct data were checked for linear amplification. Gene expression was corrected by the mean of housekeeping genes (RLP0, RLP8, and β-actin) using the ΔCt method.

### Variable Selection

Clinical characteristics considered included age at diagnosis (years); gender; Breslow depth (mm); mitotic rate level; extent of tumor infiltrating lymphocytes (absent, non-brisk, brisk); histologic type (superficial spreading, nodular, desmoplastic, lentigo maligna, acral lentiginous, spindled, dermal, spitzoid, nevoid, missed, other/unclassifiable); biopsy location (head/neck, trunk, upper extremities, lower extremities, acral); and presence of ulceration, regression, microsatellitosis, and angiolymphatic invasion.

Gene expression was determined for the following 138 genes:
ABCC3, ACTG1P20, ACVR1C, ADAM12, ADAMTS19, ADIPOQ, AGRN, ALDH2, APOD, ARSG, BCL2L11, BMBPR1B, BTC, CBX3, CD1E, CD200R1, CD274, CD44, CD68, CDH11, CDH2, CDKN1A, CDKN2A, CENPF, CNTN1, COL4A1, COL6A1, CORO2B, CPN1, CSRC, CTGF, CTLA4, CTNNB1, CXCL1, CXCL9, DCD, DLC1, EGFR, ELOVL2, ERBB2, ETV7, EYA4, FCER1A, FGFR2, FLT1, FN1, GDF11, GDF15, GPRC5B, HEY1, HHATL, HJURP, HNRNPA1P33, HOPX, IGF1R, IGFBP5, IL8, INHBA, ITGA2, ITGA3, ITGA5, ITGB1, ITGB3, ITGB5, ITGB6, ITGB7, ITGB8, KCNK10, KCNQ5, KRT14, LAMA4, LAMB1, LAMB3, LGALS1, LHFPL3, LIMA1, LOXL1, LOXL3, LOXL4, LRRC15, MITF, MKI67, MLANA, MMP10, MMP14, MMP3, MOBP, MX1, MYBL2, MYH4, MYL9, NALCN, NR2E1, NT5DC3, NUF2, OR52N4, PARM1, PDCD1, PIM2, PLAT, PLAU, PLOD3, PPP1R1A, PPP1R9A, PRAME, PRKCB, PTK2, RARA, RGS1, RLBP1, SDC3, SERPINE1, SERPINE2, SIRPG, SLC24A2, SMAD4, SPP1, SRSF11, SSX2B, TFAP2B, TGFB1, TGFB2, TGFB3, TGFBI, TGFBR1, TGFBR2, THBS1, THBS2, TMEM163, TMEM98, TNC, TOX, TP53, TUBA8, TUBB3, UPP1, VIM, WBP5

Missing expression values occurred at a rate of 0.3% of the total number of expression values (255/79212). Data were filled for regression analysis using mean imputation.

### Statistical Methods

The primary endpoint for the study was time to relapse after initial diagnosis of melanoma. Subjects who died, were lost to follow-up, or reached the end of the study period without relapse were censored at those respective timepoints. Gene expression was examined using normalized cycle threshold values (ΔCt values). All continuous variables were modeled assuming a log-linear association with time to relapse.

Multivariate models to predict time to relapse were built using Cox proportional hazards, least absolute shrinkage and selection operator (LASSO) regression with a double loop cross-validation (DLCV) component. The R glmnet package (version 4.1-1) was used to fit the LASSO models (Tibshirani, R. J R Stat Soc 1996; 58:267-88 and Tibshirani, R. Stat Med 1997; 16:385-95). Briefly, this method fits a penalized partial likelihood estimation algorithm to a Cox model model λ(t|x)=λ₀(t)exp{Xβ}, subject to Σ|βⱼ|≤s. Conditioning on the penalty term s allows for both parameter estimation and feature selection, as variables whose parameter estimates are shrunk to 0 are effectively removed from the prediction model. The number of relapses in the study (N=85) prohibited us from splitting the study set into training and testing sets. An alternative solution to this external validation approach is to internally validate the model by repeated cross-validation and average the predictive performance across the multiple test sets. This double loop cross-validation approach has been described elsewhere (Wessels et al. Bioinformatics 2005; 21:3755-62) and has been employed by us previously to predict sentinel lymph node metastasis in the study set (Bellomo et al., JCO Precision Oncology 2020:319-34). In brief, the approach (1) randomly sorts the study set; (2) uses 10-fold cross-validation to estimate the LASSO penalty term; and (3) uses 3-fold cross-validation and the penalty term from (2) to build/train a predictive model on two-thirds of the data and test the model on the remaining one-third, resulting in 3 predictive models. This process is repeated 100 times, generating a total of 300 predictive models. Due to the way the 3-fold cross-validation approach assigns subjects to training and testing sets, each subject is included in exactly 100 of the 300 model test sets. Final time-specific relapse probability estimates for a given subject are then calculated by averaging their survival estimates at various times post-diagnosis across the 100 test set models. The overall predictive capability of the model was assessed using a survival-based concordance (c-)statistic, and time-specific predictive capabilities were assessed at various years post-diagnosis using receiver operating characteristics (ROC curves). The variables most predictive of relapse were determined based on the proportion of times they were included in the 300 LASSO models. One final model was then created by fitting these most predictive variables with Cox LASSO regression using a penalty term equal to the average of the penalty terms from the 100 loops.

### Results

138 candidate biomarkers were selected for further analysis in a prospectively designed archival cohort.

The prospectively designed archival cohort comprised 681 patients with thin- and intermediate-thickness primary cutaneous melanoma who underwent an SLNb within 90 days of diagnosis. Of the 681 patients, 104 (15%) were SLN positive, in agreement with the typical prevalence in an SLNb-eligible population (Morton et al., N. Engl. J Med., 370:599-609 (2014)). Models of the likelihood of SLN metastasis were developed on the basis of clinicopathologic variables and patient age, and gene expression profiles of the primary tumor (CP-GEP models). All models were logistic regression models. Widely available CP factors considered included Breslow thickness, ulceration, mitotic rate, and patient age at diagnosis.

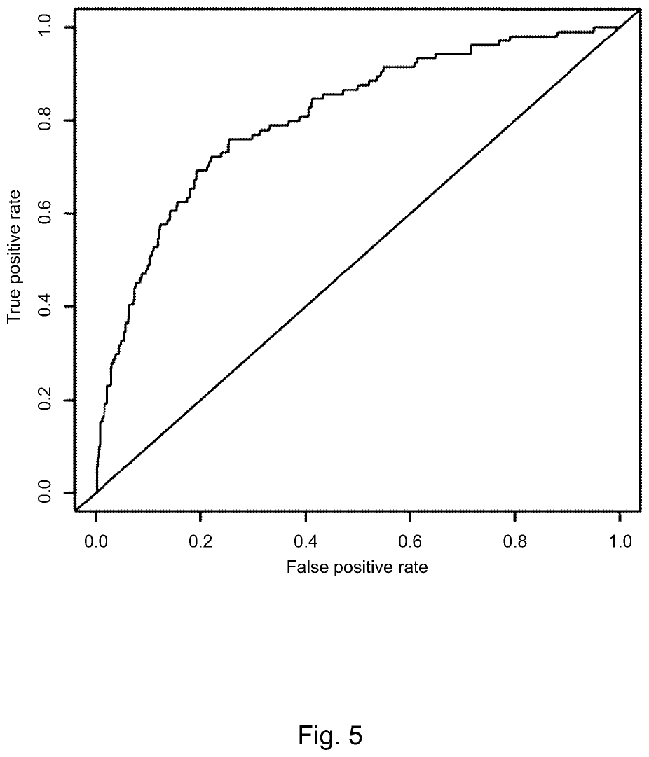

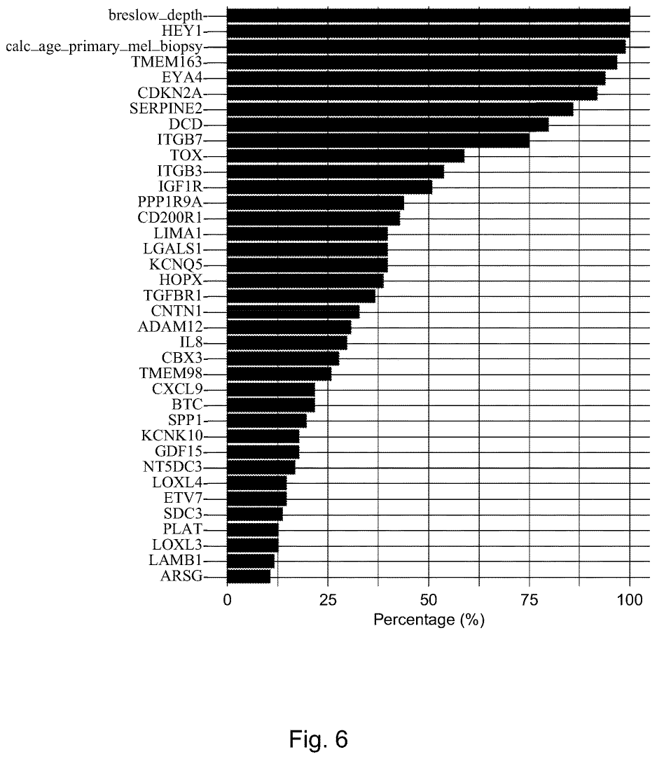

Double loop cross-validation (DLCV) and LASSO were used to identify a CP-GEP model useful for predicting SLNb metastasis. The final model had an AUC of 0.86. FIG. 5 contains a graph showing a receiver operating characteristic (ROC) from 13 variables that appeared in at least 50% of the 300 cross-validated models built. FIG. 6 shows all the variables that appeared in at least 10% of the 300 cross-validated models built. FIG. 7 lists the 13 variables that appeared in at least 50% of the cross-validated models with model coefficients.

### Example 4: Polypeptide and mRNA Levels

Levels of a TMEM163 polypeptide (and mRNA encoding the TMEM163 polypeptide), a HEY1 polypeptide (and mRNA encoding the HEY1 polypeptide), an EYA4 polypeptide (and mRNA encoding the EYA4 polypeptide), a TMEM98 polypeptide (and mRNA encoding the TMEM98 polypeptide), a RLBP1 polypeptide (and mRNA encoding the RLBP1 polypeptide), an ELOVL2 polypeptide (and mRNA encoding the ELOVL2 polypeptide), a SERPINE2 polypeptide (and mRNA encoding a SERPINE2 polypeptide), an ITGB3 polypeptide (and mRNA encoding an ITGB3 polypeptide), an IGF1R polypeptide (and mRNA encoding an IGF1R polypeptide), a CD200R1 polypeptide (and mRNA encoding the CD200R1 polypeptide), a SIRPG polypeptide (and mRNA encoding the SIRPG polypeptide), a BTC polypeptide (and mRNA encoding the BTC polypeptide), an OR52N4 polypeptide (and mRNA encoding the OR52N4 polypeptide), an ETV7 polypeptide (and mRNA encoding the ETV7 polypeptide), a TOX polypeptide (and mRNA encoding the TOX polypeptide), an ARSG polypeptide (and mRNA encoding the ARSG polypeptide), an ITGB7 polypeptide (and mRNA encoding the ITGB7 polypeptide), a CDKN2A polypeptide (and mRNA encoding a CDKN2A polypeptide), and a DCD polypeptide (and mRNA encoding a DCD polypeptide) in humans were determined.

TMEM163

| Total actin protein per cell | 7E−11 | gram |
|---|---|---|
| actin molecular weight protein | 42 | kDa |
| actin cDNA length | 1812 | bp |
| Total actin mRNA per cell | 10.5 | pg |
| molecular weight TMEM163 | 31.468 | kDa |
| TMEM163 cDNA length | 1892 | bp |

| | TMEM163 | | | |
|---|---|---|---|---|
| | ratio: | conc | total | total |
| Gene/Protein | actin/TMEM163 | TMEM163 | protein | mRNA |
| control melanoma | | | | |
| low relative to actin | 330.3946225 | 1.58739E−13 | 0.2 pg | 0.03 pg |
| high relative to actin | 124.7419616 | 4.20441E−13 | 0.4 pg | 0.09 pg |
| high-risk melanoma | | | | |
| low relative to actin | 96.54190455 | 5.43253E−13 | 0.5 pg | 0.11 pg |
| high relative to actin | 12.61759946 | 4.15663E−12 | 4.2 pg | 0.87 pg |

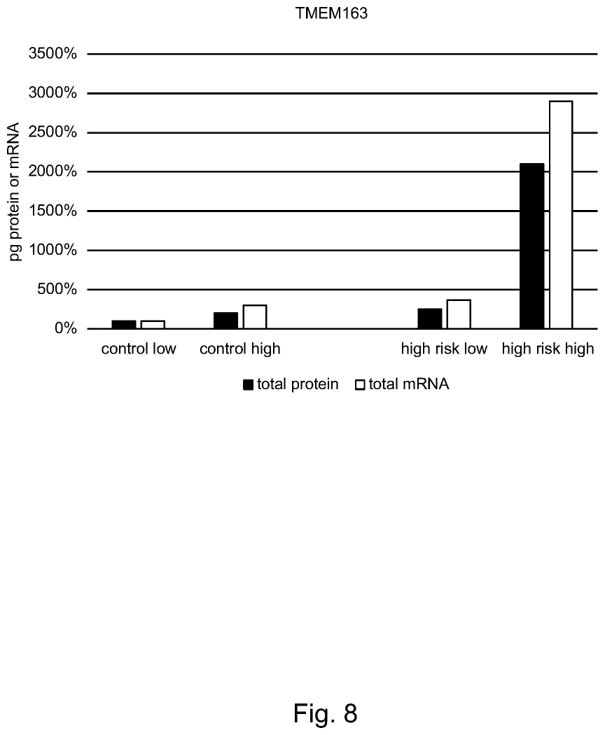

Polypeptide and mRNA levels are also shown in FIG. 8.

HEY1

| Total actin protein per cell | 7E−11 | gram |
|---|---|---|
| actin molecular weight protein | 42 | kDa |
| actin cDNA length | 1812 | bp |
| Total actin mRNA per cell | 10.5 | pg |
| molecular weight HEY1 | 33.1 | kDa |
| HEY1 cDNA length | 2296 | bp |

| | HEY1 | | | |
|---|---|---|---|---|
| | ratio: | conc | total | total |
| Gene/Protein | actin/HEY1 | HEY1 | protein | mRNA |
| control melanoma | | | | |
| low relative to actin | 99.35476961 | 5.55249E−13 | 0.6 pg | 0.13 pg |
| high relative to actin | 49.25887235 | 1.11993E−12 | 1.1 pg | 0.27 pg |
| high-risk melanoma | | | | |
| low relative to actin | 43.8525992 | 1.258E−12 | 1.3 pg | 0.3 pg |
| high relative to actin | 18.76353693 | 2.9401E−12 | 2.9 pg | 0.71 pg |

EYA4

| Total actin protein per cell | 7E−11 | gram |
|---|---|---|
| actin molecular weight protein | 42 | kDa |
| actin cDNA length | 1812 | bp |
| Total actin mRNA per cell | 10.5 | pg |
| molecular weight EYA4 | 44 | kDa |
| EYA4 cDNA length | 2569 | bp |

| | EYA4 | | | |
|---|---|---|---|---|
| | ratio: | conc | total | total |
| Gene/Protein | actin/EYA4 | EYA4 | protein | mRNA |
| control melanoma | | | | |
| low relative to actin | 238.6747643 | 3.07252E−13 | 0.3 pg | 0.06 pg |
| high relative to actin | 98.86297928 | 7.41767E−13 | 0.7 pg | 0.15 pg |
| high-risk melanoma | | | | |
| low relative to actin | 92.01780887 | 7.96947E−13 | 0.8 pg | 0.16 pg |
| high relative to actin | 23.64485551 | 3.10145E−12 | 3.1 pg | 0.63 pg |

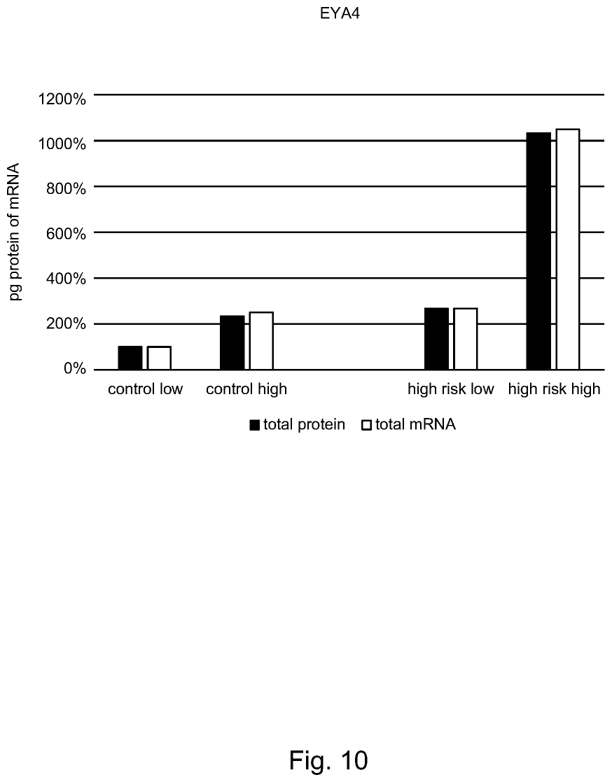

Polypeptide and mRNA levels are also shown in FIG. 10.

TMEM98

| Total actin protein per cell | 7E−11 | gram |
|---|---|---|
| actin molecular weight protein | 42 | kDa |
| actin cDNA length | 1812 | bp |
| Total actin mRNA per cell | 10.5 | pg |
| molecular weight TMEM98 | 24.6 | kDa |
| TMEM98 cDNA length | 4218 | bp |

| | TMEM98 | | | |
|---|---|---|---|---|
| | ratio: | conc | total | total |
| Gene/Protein | actin/TMEM98 | TMEM98 | protein | mRNA |
| control melanoma | | | | |
| low relative to actin | 66.42410052 | 6.17246E−13 | 0.6 pg | 0.37 pg |
| high relative to actin | 35.52286065 | 1.15419E−12 | 1.2 pg | 0.69 pg |
| high-risk melanoma | | | | |
| low relative to actin | 29.44912421 | 1.39223E−12 | 1.4 pg | 0.83 pg |
| high relative to actin | 13.63399867 | 3.00719E−12 | 3 pg | 1.79 pg |

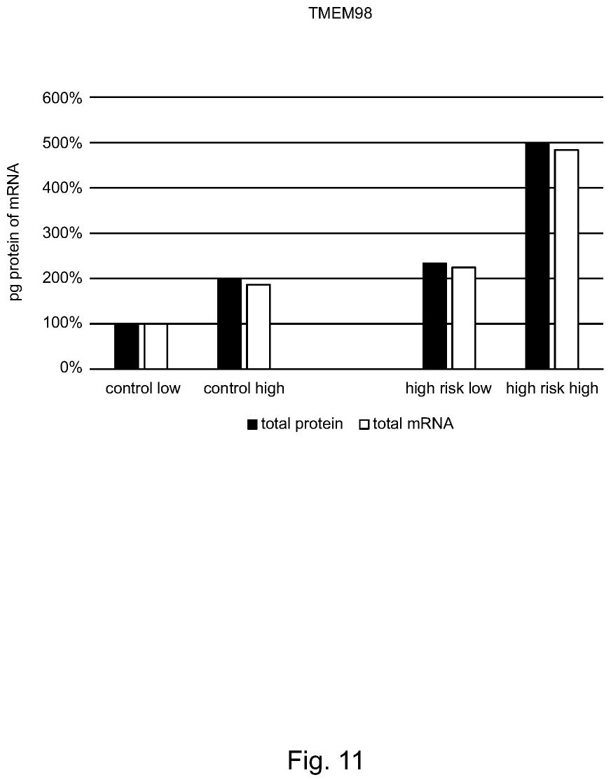

Polypeptide and mRNA levels are also shown in FIG. 11.

RLBP1

| Total actin protein per cell | 7E−11 | gram |
|---|---|---|
| actin molecular weight protein | 42 | kDa |
| actin cDNA length | 1812 | bp |
| Total actin mRNA per cell | 10.5 | pg |
| molecular weight RLBP1 | 36.5 | kDa |
| RLBP1 cDNA length | 1638 | bp |

| | RLBP1 | | | |
|---|---|---|---|---|
| | ratio: | conc | total | total |
| Gene/Protein | actin/RLBP1 | RLBP1 | protein | mRNA |
| control melanoma | | | | |
| low relative to actin | 166.6921097 | 3.64944E−13 | 0.4 pg | 0.06 pg |
| high relative to actin | 97.72454889 | 6.22498E−13 | 0.6 pg | 0.1 pg |
| high-risk melanoma | | | | |
| low relative to actin | 87.35421239 | 6.96398E−13 | 0.7 pg | 0.11 pg |
| high relative to actin | 45.16319654 | 1.34697E−12 | 1.3 pg | 0.21 pg |

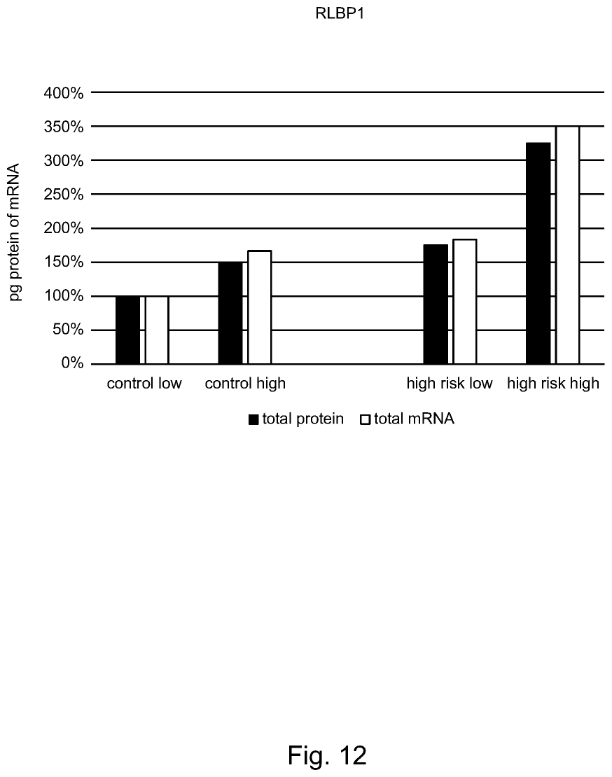

Polypeptide and mRNA levels are also shown in FIG. 12.

ELOVL2

| Total actin protein per cell | 7E−11 | gram |
|---|---|---|
| actin molecular weight protein | 42 | kDa |
| actin cDNA length | 1812 | bp |
| Total actin mRNA per cell | 10.5 | pg |
| molecular weight ELOVL2 | 34.6 | kDa |
| ELOVL2 cDNA length | 3988 | bp |

| | ELOVL2 | | | |
|---|---|---|---|---|
| | ratio: | conc | total | total |
| Gene/Protein | actin/ELOVL2 | ELOVL2 | protein | mRNA |
| control melanoma | | | | |
| low relative to actin | 160.3574505 | 3.59613E−13 | 0.4 pg | 0.14 pg |
| high relative to actin | 65.55286188 | 8.79697E−13 | 0.9 pg | 0.35 pg |
| high-risk melanoma | | | | |
| low relative to actin | 52.45441831 | 1.09937E−12 | 1.1 pg | 0.44 pg |
| high relative to actin | 9.61930638 | 5.99489E−12 | 6 pg | 2.4 pg |

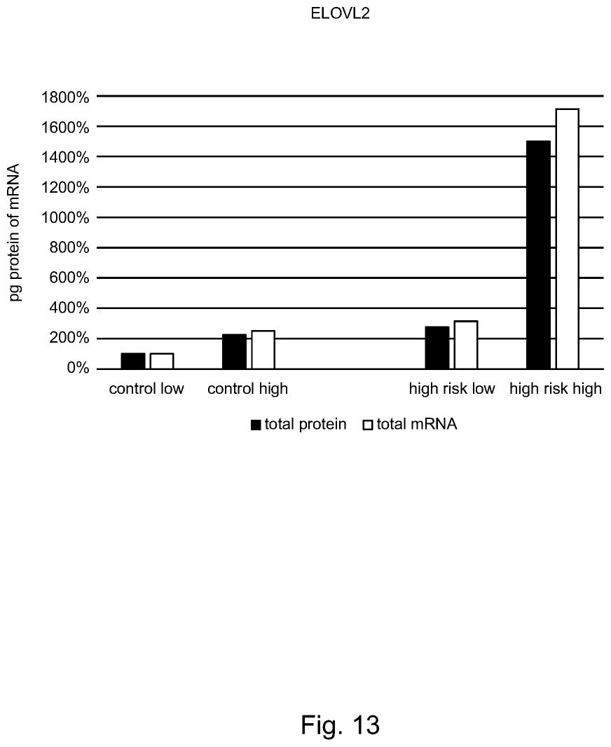

Polypeptide and mRNA levels are also shown in FIG. 13.

SERPINE2

| Total actin protein per cell | 7E−11 | gram |
|---|---|---|
| actin molecular weight protein | 42 | kDa |
| actin cDNA length | 1812 | bp |
| Total actin mRNA per cell | 10.5 | pg |
| molecular weight SERPINE2 | 44 | kDa |
| SERPINE2 cDNA length | 2569 | bp |

| | SERPINE2 | | | |
|---|---|---|---|---|
| | ratio: | conc | total | total |
| Gene/Protein | actin/SERPINE2 | SERPINE2 | protein | mRNA |
| control melanoma | | | | |
| low relative to actin | 36.87940359 | 1.98846E−12 | 2 pg | 0.4 pg |
| high relative to actin | 2.720222302 | 2.69586E−11 | 27 pg | 5.47 pg |
| high-risk melanoma | | | | |
| low relative to actin | 0.406225346 | 1.80524E−10 | 180.5 pg | 36.65 pg |
| high relative to actin | 4.679524541 | 3.43165E−10 | 343.2 pg | 69.66 pg |

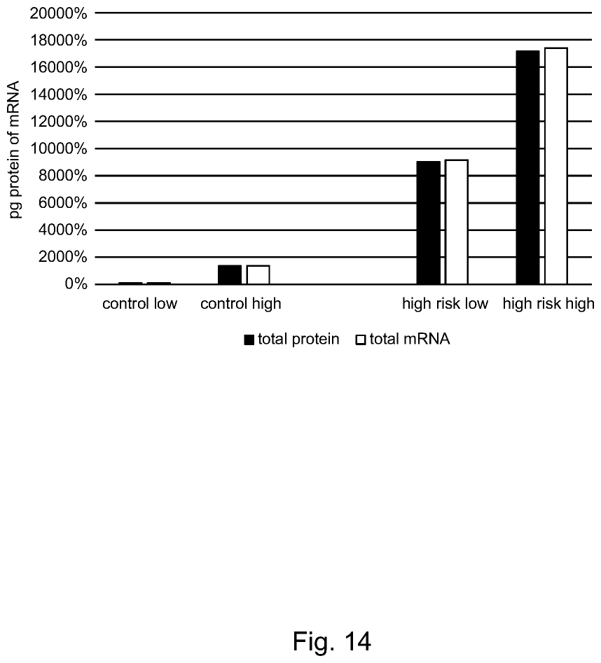

Polypeptide and mRNA levels are also shown in FIG. 14.

ITGB3

| Total actin protein per cell | 7E−11 | gram |
|---|---|---|
| actin molecular weight protein | 42 | kDa |
| actin cDNA length | 1812 | bp |
| Total actin mRNA per cell | 10.5 | pg |
| molecular weight ITGB3 | 87.1 | kDa |
| ITGB3 cDNA length | 5941 | bp |

| | ITGB3 | | | |
|---|---|---|---|---|
| | ratio: | conc | total | total |
| Gene/Protein | actin/ITGB3 | ITGB3 | protein | mRNA |
| control melanoma | | | | |
| low relative to actin | 101.1362689 | 1.43536E−12 | 1.4 pg | 0.34 pg |
| high relative to actin | 46.65531886 | 3.11147E−12 | 3.1 pg | 0.74 pg |
| high-risk melanoma | | | | |
| low relative to actin | 39.00591519 | 3.72166E−12 | 3.7 pg | 0.88 pg |
| high relative to actin | 6.518705678 | 2.22692E−11 | 22.3 pg | 5.28 pg |

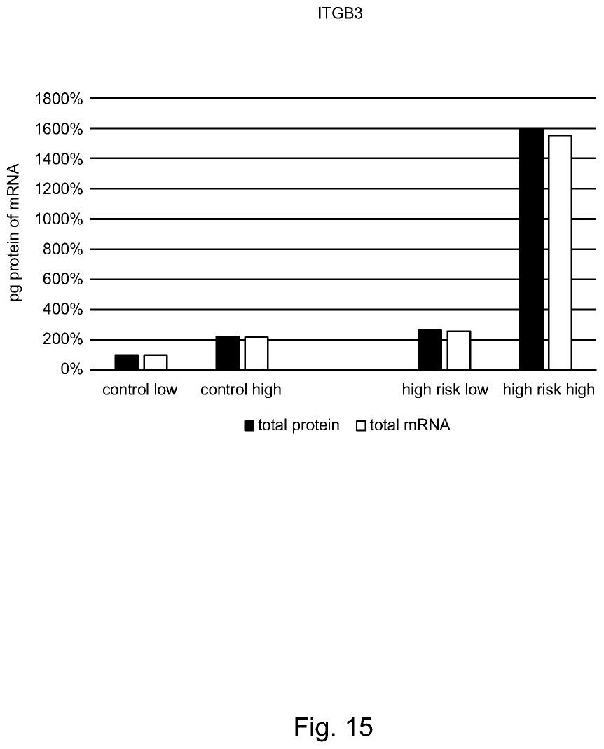

Polypeptide and mRNA levels are also shown in FIG. 15.

IGF1R

| Total actin protein per cell | 7E−11 | gram |
|---|---|---|
| actin molecular weight protein | 42 | kDa |
| actin cDNA length | 1812 | bp |
| Total actin mRNA per cell | 10.5 | pg |
| molecular weight IGF1R | 154.8 | kDa |
| IGF1R cDNA length | 12235 | bp |

| | IGF1R | | | |
|---|---|---|---|---|
| | ratio: | conc | total | total |
| Gene/Protein | actin/IGF1R | IGF1R | protein | mRNA |
| control melanoma | | | | |
| low relative to actin | 43.24812558 | 5.96558E−12 | 6 pg | 1.64 pg |
| high relative to actin | 25.30763912 | 1.01946E−11 | 10.2 pg | 2.8 pg |
| high-risk melanoma | | | | |
| low relative to actin | 23.04840816 | 1.11938E−11 | 11.2 pg | 3.08 pg |
| high relative to actin | 12.07941982 | 2.13586E−11 | 21.4 pg | 5.87 pg |

Polypeptide and mRNA levels are also shown in FIG. 15.

CD200R1

| Total actin protein per cell | 7E−11 | gram |
|---|---|---|
| actin molecular weight protein | 42 | kDa |
| actin cDNA length | 1812 | bp |
| Total actin mRNA per cell | 10.5 | pg |
| molecular weight CD200R1 | 39 | kDa |
| CD200R1 cDNA length | 3765 | bp |

| | CD200R1 | | | |
|---|---|---|---|---|
| | ratio: | conc | total | total |
| Gene/Protein | actin/CD200R1 | CD200R1 | protein | mRNA |
| control melanoma | | | | |
| low relative to actin | 61.45569447 | 1.05767E−12 | 1.1 pg | 0.36 pg |
| high relative to actin | 36.04537465 | 1.80328E−12 | 1.8 pg | 0.61 pg |
| high-risk melanoma | | | | |
| low relative to actin | 122.5091864 | 5.30572E−13 | 0.5 pg | 0.18 pg |
| high relative to actin | 66.69726873 | 9.74553E−13 | 1 pg | 0.33 pg |

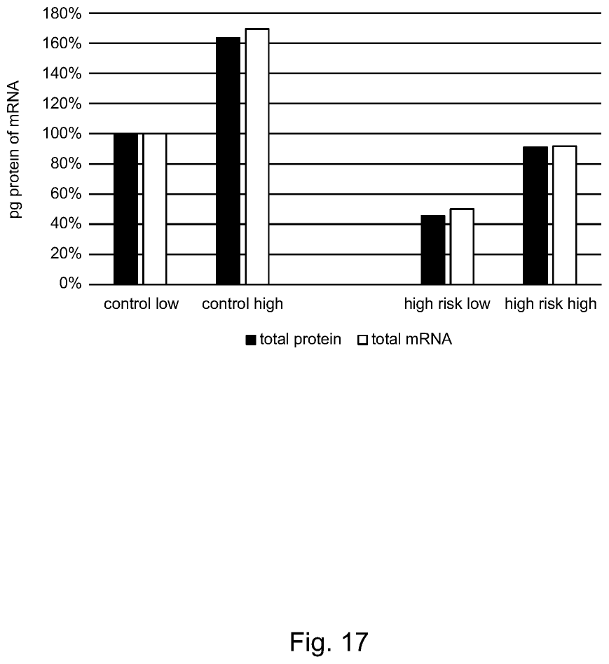

Polypeptide and mRNA levels are also shown in FIG. 17.

SIRPG

| Total actin protein per cell | 7E−11 | gram |
|---|---|---|
| actin molecular weight protein | 42 | kDa |
| actin cDNA length | 1812 | bp |
| Total actin mRNA per cell | 10.5 | pg |
| molecular weight SIRPG | 42.5 | kDa |
| SIRPG cDNA length | 1716 | bp |

| | SIRPG | | | |
|---|---|---|---|---|
| | ratio: | conc | total | total |
| Gene/Protein | actin/SIRPG | SIRPG | protein | mRNA |
| control melanoma | | | | |
| low relative to actin | 30.20109708 | 2.34539E−12 | 2.3 pg | 0.33 pg |
| high relative to actin | 19.97510107 | 3.54608E−12 | 3.5 pg | 0.5 pg |
| high-risk melanoma | | | | |
| low relative to actin | 48.89204071 | 1.44877E−12 | 1.4 pg | 0.2 pg |
| high relative to actin | 29.74006437 | 2.38175E−12 | 2.4 pg | 0.33 pg |

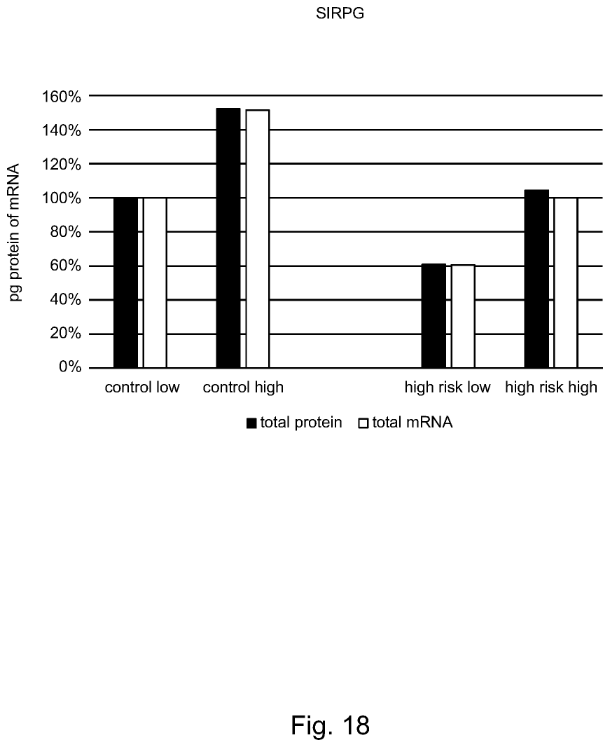

Polypeptide and mRNA levels are also shown in FIG. 18.

BTC

| Total actin protein per cell | 7E−11 | gram |
|---|---|---|
| actin molecular weight protein | 42 | kDa |
| actin cDNA length | 1812 | bp |
| Total actin mRNA per cell | 10.5 | pg |
| molecular weight BTC | 19.8 | kDa |
| BTC cDNA length | 2653 | bp |

| | BTC | | | |
|---|---|---|---|---|
| | ratio: | conc | total | total |
| Gene/Protein | actin/BTC | BTC | protein | mRNA |
| control melanoma | | | | |
| low relative to actin | 63.58285384 | 5.19008E−13 | 0.5 pg | 0.24 pg |
| high relative to actin | 23.53093971 | 1.40241E−12 | 1.4 pg | 0.65 pg |
| high-risk melanoma | | | | |
| low relative to actin | 180.7477467 | 1.82575E−13 | 0.2 pg | 0.09 pg |
| high relative to actin | 67.36963644 | 4.89835E−13 | 0.5 pg | 0.23 pg |

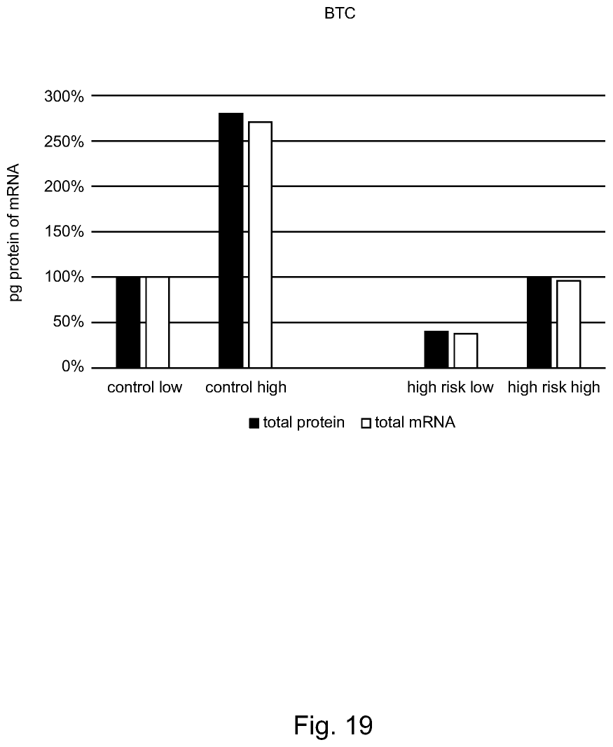

Polypeptide and mRNA levels are also shown in FIG. 19.

OR52N4

| Total actin protein per cell | 7E−11 | gram |
|---|---|---|
| actin molecular weight protein | 42 | kDa |
| actin cDNA length | 1812 | bp |
| Total actin mRNA per cell | 10.5 | pg |
| molecular weight OR52N4 | 36.1 | kDa |
| OR52N4 cDNA length | 1329 | bp |

| | OR52N4 | | | |
|---|---|---|---|---|
| | ratio: | conc | total | total |
| Gene/Protein | actin/OR52N4 | OR52N4 | protein | mRNA |
| control melanoma | | | | |
| low relative to actin | 82.3572745 | 7.30557E−13 | 0.7 pg | 0.09 pg |
| high relative to actin | 40.8644394 | 1.47235E−12 | 1.5 pg | 0.19 pg |
| high-risk melanoma | | | | |
| low relative to actin | 179.6006283 | 3.35003E−13 | 0.3 pg | 0.04 pg |
| high relative to actin | 87.39319047 | 6.88459E−13 | 0.7 pg | 0.09 pg |

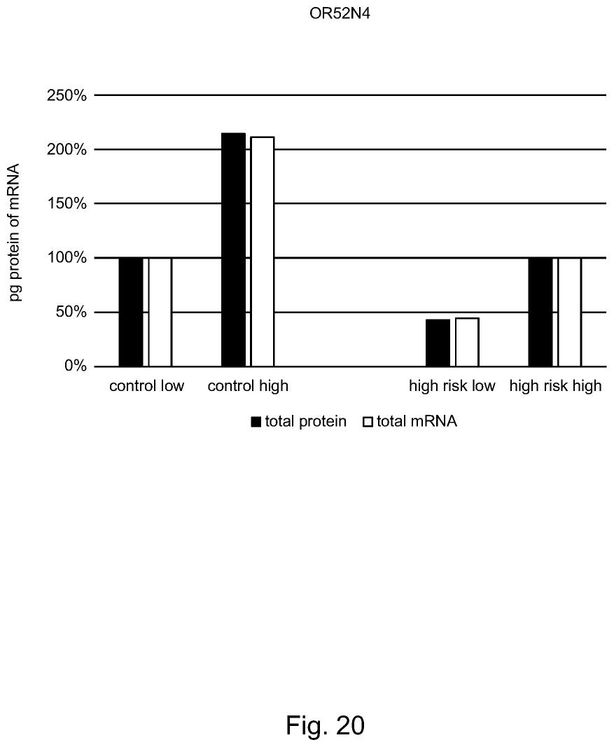

Polypeptide and mRNA levels are also shown in FIG. 20.

ETV7

| Total actin protein per cell | 7E−11 | gram |
|---|---|---|
| actin molecular weight protein | 42 | kDa |
| actin cDNA length | 1812 | bp |
| Total actin mRNA per cell | 10.5 | pg |
| molecular weight ETV7 | 36.2 | kDa |
| ETV7 cDNA length | 1530 | bp |

| | ETV7 | | | |
|---|---|---|---|---|
| | ratio: | conc | total | total |
| Gene/Protein | actin/ETV7 | ETV7 | protein | mRNA |
| control melanoma | | | | |
| low relative to actin | 36.64661584 | 1.64635E−12 | 1.6 pg | 0.24 pg |
| high relative to actin | 16.93749804 | 3.56212E−12 | 3.6 pg | 0.52 pg |
| high-risk melanoma | | | | |
| low relative to actin | 86.14760916 | 7.00348E−13 | 0.7 pg | 0.1 pg |
| high relative to actin | 38.14222915 | 1.5818E−12 | 1.6 pg | 0.23 pg |

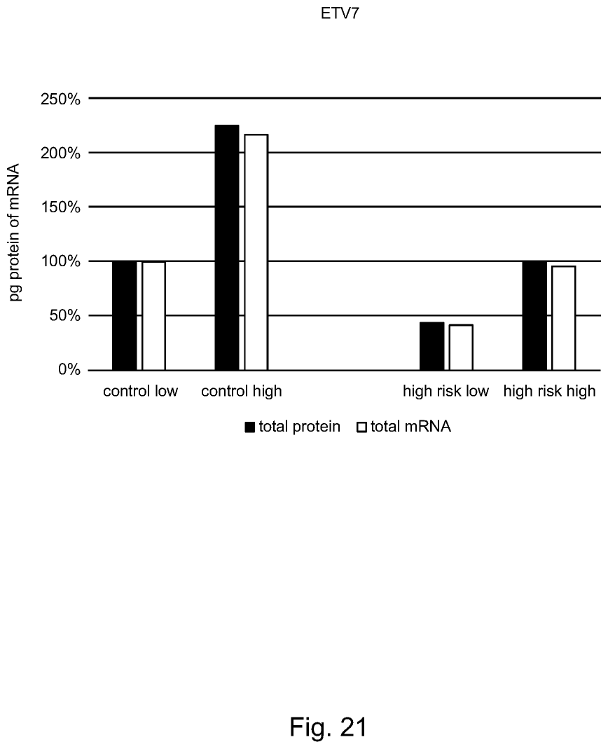

Polypeptide and mRNA levels are also shown in FIG. 21.

TOX

| Total actin protein per cell | 7E−11 | gram |
|---|---|---|
| actin molecular weight protein | 42 | kDa |
| actin cDNA length | 1812 | bp |
| Total actin mRNA per cell | 10.5 | pg |
| molecular weight TOX | 57.5 | kDa |
| TOX cDNA length | 4076 | bp |

| | TOX | | | |
|---|---|---|---|---|
| | ratio: | conc | total | total |
| Gene/Protein | actin/TOX | TOX | protein | mRNA |
| control melanoma | | | | |
| low relative to actin | 58.26103173 | 1.6449E−12 | 1.6 pg | 0.41 pg |
| high relative to actin | 30.94344811 | 3.09705E−12 | 3.1 pg | 0.76 pg |
| high-risk melanoma | | | | |
| low relative to actin | 121.863074 | 7.86402E−13 | 0.8 pg | 0.19 pg |
| high relative to actin | 60.21725666 | 1.59146E−12 | 1.6 pg | 0.39 pg |

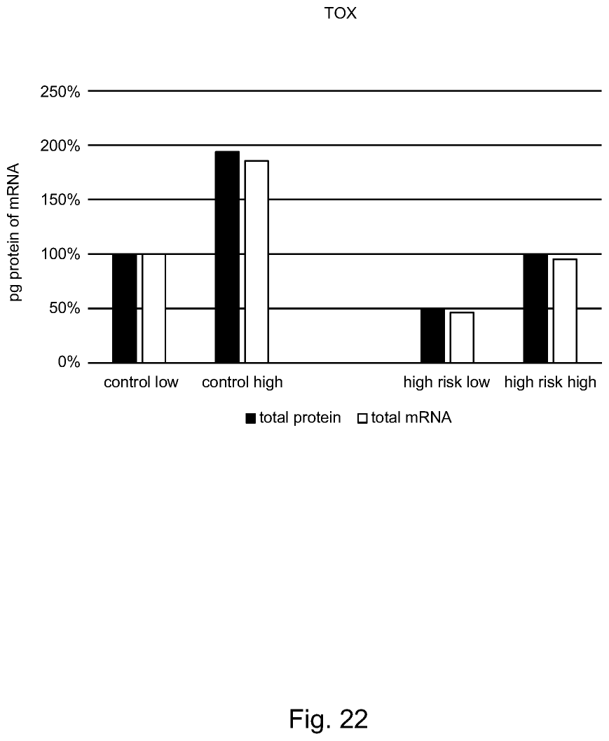

Polypeptide and mRNA levels are also shown in FIG. 22.

ARSG

| Total actin protein per cell | 7E−11 | gram |
|---|---|---|
| actin molecular weight protein | 42 | kDa |
| actin cDNA length | 1812 | bp |
| Total actin mRNA per cell | 10.5 | pg |
| molecular weight ARSG | 57 | kDa |
| ARSG cDNA length | 4642 | bp |

| | ARSG | | | |
|---|---|---|---|---|
| | ratio: | conc | total | total |
| Gene/Protein | actin/ARSG | ARSG | protein | mRNA |
| control melanoma | | | | |
| low relative to actin | 47.33842259 | 2.00683E−12 | 2 pg | 0.57 pg |
| high relative to actin | 31.90040492 | 2.97802E−12 | 3 pg | 0.84 pg |
| high-risk melanoma | | | | |
| low relative to actin | 82.06920049 | 1.15756E−12 | 1.2 pg | 0.33 pg |
| high relative to actin | 49.20319755 | 1.93077E−12 | 1.9 pg | 0.55 pg |

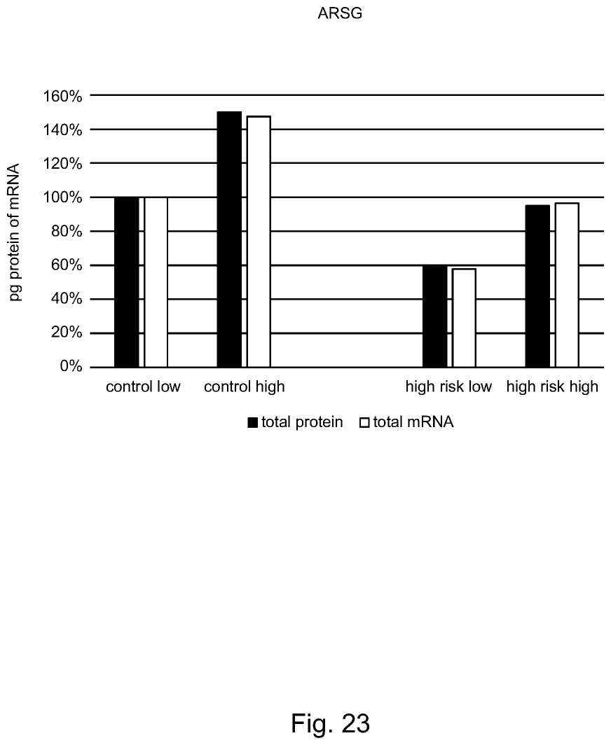

Polypeptide and mRNA levels are also shown in FIG. 23.

ITGB7

| Total actin protein per cell | 7E−11 | gram |
|---|---|---|
| actin molecular weight protein | 42 | kDa |
| actin cDNA length | 1812 | bp |
| Total actin mRNA per cell | 10.5 | pg |
| molecular weight ITGB7 | 87 | kDa |
| ITGB7 cDNA length | 2806 | bp |

| | ITGB7 | | | |
|---|---|---|---|---|
| | ratio: | conc | total | total |
| Gene/Protein | actin/ITGB7 | ITGB7 | protein | mRNA |
| control melanoma | | | | |
| low relative to actin | 54.53799755 | 2.6587E−12 | 2.7 pg | 0.3 pg |
| high relative to actin | 31.63274711 | 4.58386E−12 | 4.6 pg | 0.51 pg |
| high-risk melanoma | | | | |
| low relative to actin | 100.8127255 | 1.43831E−12 | 1.4 pg | 0.16 pg |
| high relative to actin | 57.30050441 | 2.53052E−12 | 2.5 pg | 0.28 pg |

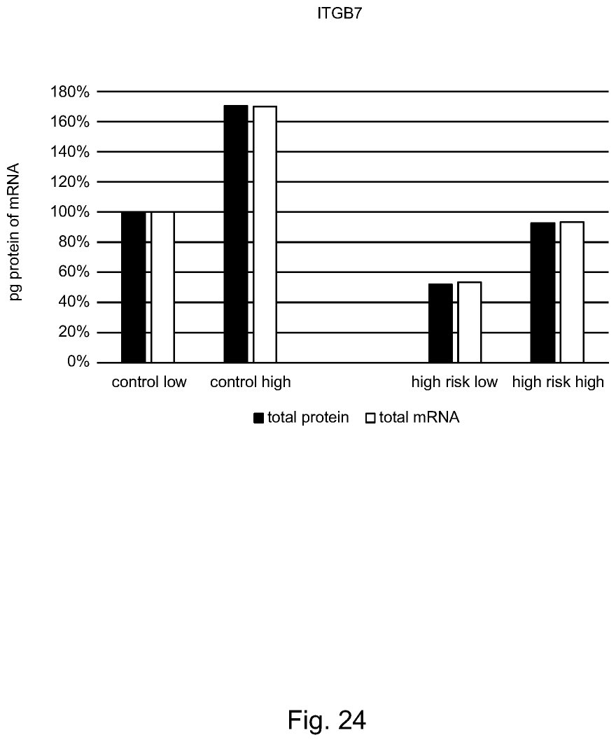

Polypeptide and mRNA levels are also shown in FIG. 24.

CDKN2A

| Total actin protein per cell | 7E−11 | gram |
|---|---|---|
| actin molecular weight protein | 42 | kDa |
| actin cDNA length | 1812 | bp |
| Total actin mRNA per cell | 10.5 | pg |
| molecular weight CDKN2A | 14 | kDa |
| CDKN2A cDNA length | 1052 | bp |

| | CDKN2A | | | |
|---|---|---|---|---|
| | ratio: | conc | total | total |
| Gene/Protein | actin/CDKN2A | CDKN2A | protein | mRNA |
| control melanoma | | | | |
| low relative to actin | 93.14932531 | 2.50494E−13 | 0.3 pg | 0.07 pg |
| high relative to actin | 35.96395697 | 6.48798E−13 | 0.6 pg | 0.17 pg |
| high-risk melanoma | | | | |
| low relative to actin | 289.13282 | 8.07011E−14 | 0.1 pg | 0.02 pg |
| high relative to actin | 102.1624666 | 2.28394E−13 | 0.2 pg | 0.06 pg |

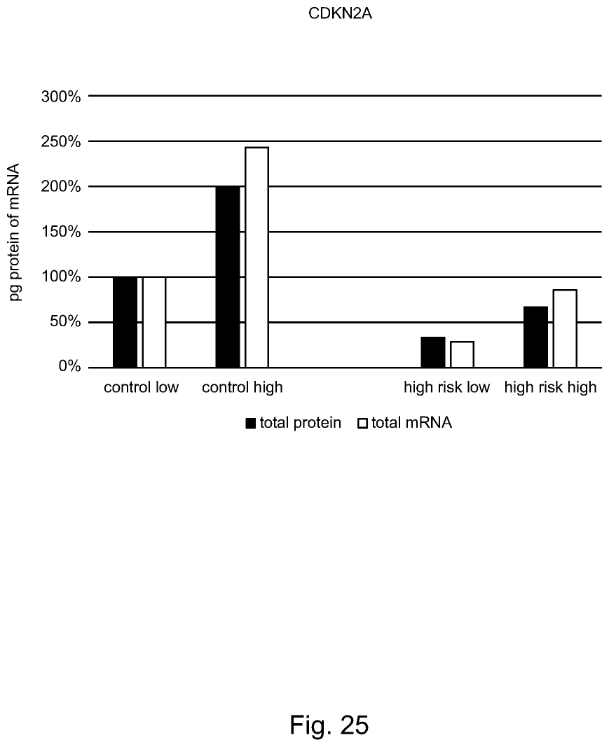

Polypeptide and mRNA levels are also shown in FIG. 25.

DCD

| Total actin protein per cell | 7E−11 | gram |
|---|---|---|
| actin molecular weight protein | 42 | kDa |
| actin cDNA length | 1812 | bp |
| Total actin mRNA per cell | 10.5 | pg |
| molecular weight DCD | 11.3 | kDa |
| DCD cDNA length | 519 | bp |

| | DCD | | | | | |
|---|---|---|---|---|---|---|
| | ratio: | conc | total | | total | |
| Gene/Protein | actin/DCD | DCD | protein | | mRNA | |
| control melanoma | | | | | | |
| low relative to actin | 30.62231331 | 6.1502E−13 | 0.6 | pg | 0.1 | pg |
| high relative to actin | 90.42236757 | 1.70295E−09 | 1703 | pg | 271.94 | pg |
| high-risk melanoma | | | | | | |
| low relative to actin | 734.8214103 | 2.56298E−14 | 0.03 | pg | 0.004 | pg |
| high relative to actin | 33.26690615 | 5.66128E−13 | 0.6 | pg | 0.09 | pg |

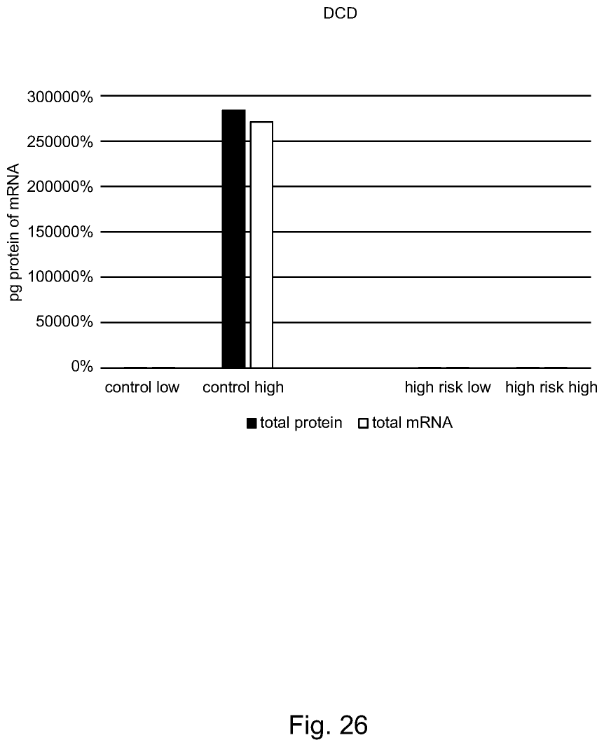

Polypeptide and mRNA levels are also shown in FIG. 26.

### Example 5: Treating PCM

A tissue sample containing one or more melanoma cells is obtained from a human having PCM. The obtained sample is examined for the presence or absence of (a) an altered level of a TMEM163 polypeptide (or mRNA encoding a TMEM163 polypeptide), a HEY1 polypeptide (or mRNA encoding a HEY1 polypeptide), an EYA4 polypeptide (or mRNA encoding an EYA4 polypeptide), a TMEM98 polypeptide (or mRNA encoding a TMEM98 polypeptide), a RLBP1 polypeptide (or mRNA encoding a RLBP1 polypeptide), an ELOVL2 polypeptide (or mRNA encoding an ELOVL2 polypeptide), a SERPINE2 polypeptide (or mRNA encoding a SERPINE2 polypeptide), an ITGB3 polypeptide (or mRNA encoding an ITGB3 polypeptide), an IGF1R polypeptide (or mRNA encoding an IGF1R polypeptide), a CD200R1 polypeptide (or mRNA encoding a CD200R1 polypeptide), a SIRPG polypeptide (or mRNA encoding a SIRPG polypeptide), a BTC polypeptide (or mRNA encoding a BTC polypeptide), an OR52N4 polypeptide (or mRNA encoding an OR52N4 polypeptide), an ETV7 polypeptide (or mRNA encoding an ETV7 polypeptide), a TOX polypeptide (or mRNA encoding a TOX polypeptide), an ARSG polypeptide (or mRNA encoding an ARSG polypeptide), an ITGB7 polypeptide (or mRNA encoding an ITGB7 polypeptide), a decreased level of a CDKN2A polypeptide (or mRNA encoding a CDKN2A polypeptide), and a decreased level of a DCD polypeptide (or mRNA encoding a DCD polypeptide); and (b) one or more mutations in a BRAF polypeptide and/or a NRAS polypeptide.

If the presence of (a) an increased level of a TMEM163 polypeptide (or mRNA encoding a TMEM163 polypeptide), an increased level of a HEY1 polypeptide (or mRNA encoding a HEY1 polypeptide), an increased level of an EYA4 polypeptide (or mRNA encoding an EYA4 polypeptide), an increased level of a TMEM98 polypeptide (or mRNA encoding a TMEM98 polypeptide), an increased level of a RLBP1 polypeptide (or mRNA encoding a RLBP1 polypeptide), an increased level of an ELOVL2 polypeptide (or mRNA encoding an ELOVL2 polypeptide), an increased level of a SERPINE2 polypeptide (or mRNA encoding a SERPINE2 polypeptide), an increased level of an ITGB3 polypeptide (or mRNA encoding an ITGB3 polypeptide), an increased level of an IGF1R polypeptide (or mRNA encoding an IGF1R polypeptide), a decreased level of a CD200R1 polypeptide (or mRNA encoding a CD200R1 polypeptide), a decreased level of a SIRPG polypeptide (or mRNA encoding a SIRPG polypeptide), a decreased level of a BTC polypeptide (or mRNA encoding a BTC polypeptide), a decreased level of an OR52N4 polypeptide (or mRNA encoding an OR52N4 polypeptide), a decreased level of an ETV7 polypeptide (or mRNA encoding an ETV7 polypeptide), a decreased level of a TOX polypeptide (or mRNA encoding a TOX polypeptide), a decreased level of an ARSG polypeptide (or mRNA encoding an ARSG polypeptide), a decreased level of an ITGB7 polypeptide (or mRNA encoding an ITGB7 polypeptide), a decreased level of a CDKN2A polypeptide (or mRNA encoding a CDKN2A polypeptide), and/or a decreased level of a DCD polypeptide (or mRNA encoding a DCD polypeptide); and/or (b) a BRAFV600 substitution and/or a NRASQ61 substitution is detected in the sample, then the PCM is identified as being likely to metastasize (e.g., likely to metastasize to one or more lymph nodes), and the human is subjected to surgery to remove the PCM and is administered one or more adjuvant therapies.

The surgery in combination with the one or more adjuvant therapies can reduce number of cancer cells within the human while minimizing the risk of metastasis.

### Example 6: Treating PCM

A tissue sample containing one or more melanoma cells is obtained from a human having PCM. The obtained sample is examined for the presence or absence of (a) an altered level of a TMEM163 polypeptide (or mRNA encoding a TMEM163 polypeptide), a HEY1 polypeptide (or mRNA encoding a HEY1 polypeptide), an EYA4 polypeptide (or mRNA encoding an EYA4 polypeptide), a TMEM98 polypeptide (or mRNA encoding a TMEM98 polypeptide), a RLBP1 polypeptide (or mRNA encoding a RLBP1 polypeptide), an ELOVL2 polypeptide (or mRNA encoding an ELOVL2 polypeptide), a SERPINE2 polypeptide (or mRNA encoding a SERPINE2 polypeptide), an ITGB3 polypeptide (or mRNA encoding an ITGB3 polypeptide), an IGF1R polypeptide (or mRNA encoding an IGF1R polypeptide), a CD200R1 polypeptide (or mRNA encoding a CD200R1 polypeptide), a SIRPG polypeptide (or mRNA encoding a SIRPG polypeptide), a BTC polypeptide (or mRNA encoding a BTC polypeptide), an OR52N4 polypeptide (or mRNA encoding an OR52N4 polypeptide), an ETV7 polypeptide (or mRNA encoding an ETV7 polypeptide), a TOX polypeptide (or mRNA encoding a TOX polypeptide), an ARSG polypeptide (or mRNA encoding an ARSG polypeptide), an ITGB7 polypeptide (or mRNA encoding an ITGB7 polypeptide), a decreased level of a CDKN2A polypeptide (or mRNA encoding a CDKN2A polypeptide), and a decreased level of a DCD polypeptide (or mRNA encoding a DCD polypeptide); and (b) one or more mutations in a BRAF polypeptide and/or a NRAS polypeptide.

If the absence of (a) an increased level of a TMEM163 polypeptide (or mRNA encoding a TMEM163 polypeptide), an increased level of a HEY1 polypeptide (or mRNA encoding a HEY1 polypeptide), an increased level of an EYA4 polypeptide (or mRNA encoding an EYA4 polypeptide), an increased level of a TMEM98 polypeptide (or mRNA encoding a TMEM98 polypeptide), an increased level of a RLBP1 polypeptide (or mRNA encoding a RLBP1 polypeptide), an increased level of an ELOVL2 polypeptide (or mRNA encoding an ELOVL2 polypeptide), an increased level of a SERPINE2 polypeptide (or mRNA encoding a SERPINE2 polypeptide), an increased level of an ITGB3 polypeptide (or mRNA encoding an ITGB3 polypeptide), an increased level of an IGF1R polypeptide (or mRNA encoding an IGF1R polypeptide), a decreased level of a CD200R1 polypeptide (or mRNA encoding a CD200R1 polypeptide), a decreased level of a SIRPG polypeptide (or mRNA encoding a SIRPG polypeptide), a decreased level of a BTC polypeptide (or mRNA encoding a BTC polypeptide), a decreased level of an OR52N4 polypeptide (or mRNA encoding an OR52N4 polypeptide), a decreased level of an ETV7 polypeptide (or mRNA encoding an ETV7 polypeptide), a decreased level of a TOX polypeptide (or mRNA encoding a TOX polypeptide), a decreased level of an ARSG polypeptide (or mRNA encoding an ARSG polypeptide), a decreased level of an ITGB7 polypeptide (or mRNA encoding an ITGB7 polypeptide), a decreased level of a CDKN2A polypeptide (or mRNA encoding a CDKN2A polypeptide), and a decreased level of a DCD polypeptide (or mRNA encoding a DCD polypeptide); and (b) a BRAFV600 substitution and a NRASQ61 substitution is detected in the sample, then the PCM is identified as not being likely to metastasize (e.g., likely to metastasize to one or more lymph nodes), and the human is subjected to surgery to remove the PCM without any adjuvant therapy.

The surgery can reduce number of cancer cells within the human while sparing the human from unnecessary risks and/or complications associated with SNL biopsies.

### OTHER EMBODIMENTS

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

1. A method for assessing a mammal having a primary cutaneous melanoma (PCM), wherein the method comprises:
(a) determining if a sample from the mammal contains a presence or absence of (1) an increased level of a transmembrane Protein 163 polypeptide or mRNA encoding the TMEM163 polypeptide, (2) an increased level of a hairy/enhancer-of-split related with YRPW motif protein 1 (HEY1) polypeptide or mRNA encoding the HEY1 polypeptide, (3) an increased level of an EYA transcriptional coactivator and phosphatase 4 (EYA4) polypeptide or mRNA encoding the EYA4 polypeptide, (4) an increased level of a transmembrane protein 98 (TMEM98) polypeptide or mRNA encoding the TMEM98 polypeptide, (5) an increased level of a retinaldehyde binding protein 1 polypeptide or mRNA encoding the RLBP1 polypeptide, (6) an increased level of an elongation of very-long-chain fatty acids-like 2 (ELOVL2) polypeptide or mRNA encoding the ELOVL2 polypeptide, (7) an increased level of a serpin family E member 2 (SERPINE2) polypeptide or mRNA encoding the SERPINE2 polypeptide, (8) an increased level of an integrin subunit beta 3 polypeptide or mRNA encoding the ITGB3 polypeptide, (9) an increased level of an insulin like growth factor 1 receptor (IGF1R) polypeptide or mRNA encoding the IGF1R polypeptide, (10) a decreased level of a cell surface glycoprotein CD200 receptor 1 (CD200R1) polypeptide or mRNA encoding the CD200R1 polypeptide, (11) a decreased level of a signal regulatory protein gamma (SIRPG) polypeptide or mRNA encoding the SIRPG polypeptide, (12) a decreased level of a betacellulin (BTC) polypeptide or mRNA encoding the BTC polypeptide, (13) a decreased level of an olfactory receptor family 52 subfamily N member 4 (OR52N4) polypeptide or mRNA encoding the OR52N4 polypeptide, (14) a decreased level of an ETS variant transcription factor 7 (ETV7) polypeptide or mRNA encoding the ETV7 polypeptide, (15) a decreased level of a thymocyte selection-associated high mobility group box (TOX) polypeptide or mRNA encoding the TOX polypeptide, (16) a decreased level of an arylsulfatase G (ARSG) polypeptide or mRNA encoding the ARSG polypeptide, (17) a decreased level of an integrin subunit beta 7 (ITGB7) polypeptide or mRNA encoding the ITGB7 polypeptide, (18) a decreased level of a cyclin dependent kinase inhibitor 2A (CDKN2A) polypeptide or mRNA encoding the CDKN2A polypeptide, or (19) a decreased level of a dermcidin (DCD) polypeptide or mRNA encoding the DCD polypeptide;
(b) classifying the PCM as being likely to metastasize if a presence of at least 11 of (1)-(19) are determined; and
(c) classifying the PCM as not being likely to metastasize if the absence of each of (1)-(19) is determined.

2.-33. (canceled)

34. A method for selecting a treatment for a mammal having a primary cutaneous melanoma (PCM), wherein the method comprises:
(a) determining that a sample from the mammal contains a presence of at least 11 of: (1) an increased level of a TMEM163 polypeptide or mRNA encoding the TMEM163 polypeptide, (2) an increased level of a HEY1 polypeptide or mRNA encoding the HEY1 polypeptide, (3) an increased level of an EYA4 polypeptide or mRNA encoding the EYA4 polypeptide, (4) an increased level of a TMEM98 polypeptide or mRNA encoding the TMEM98 polypeptide, (5) an increased level of a RLBP1 polypeptide or mRNA encoding the RLBP1 polypeptide, (6) an increased level of an ELOVL2 polypeptide or mRNA encoding the ELOVL2 polypeptide, (7) an increased level of a SERPINE2 polypeptide or mRNA encoding the SERPINE2 polypeptide, (8) an increased level of an ITGB3 polypeptide or mRNA encoding the ITGB3 polypeptide, (9) an increased level of an IGF1R polypeptide or mRNA encoding the IGF1R polypeptide, (10) a decreased level of a CD200R1 polypeptide or mRNA encoding the CD200R1 polypeptide, (11) a decreased level of a SIRPG polypeptide or mRNA encoding the SIRPG polypeptide, (12) a decreased level of a BTC polypeptide or mRNA encoding the BTC polypeptide, (13) a decreased level of an OR52N4 polypeptide or mRNA encoding the OR52N4 polypeptide, (14) a decreased level of an ETV7 polypeptide or mRNA encoding the ETV7 polypeptide, (15) a decreased level of a TOX polypeptide or mRNA encoding the TOX polypeptide, (16) a decreased level of an ARSG polypeptide or mRNA encoding the ARSG polypeptide, (17) a decreased level of an ITGB7 polypeptide or mRNA encoding the ITGB7 polypeptide, (18) a decreased level of a CDKN2A polypeptide or mRNA encoding the CDKN2A polypeptide, and (19) a decreased level of a DCD polypeptide or mRNA encoding the DCD polypeptide, wherein the sample is a tissue sample comprising a melanoma cell; and
(b) selecting the mammal for surgery to remove the PCM and to receive an adjuvant therapy.

35. The method of claim 34, the method further comprising determining that the PCM has a Breslow depth of from about 0.8 mm to about 10 mm.

36. The method of claim 34, the method further comprising determining that the sample contains a presence of a mutation in a BRAF polypeptide or a mutation in a NRAS polypeptide.

37. A method for selecting a treatment for a mammal having a primary cutaneous melanoma (PCM), wherein the method comprises:
(a) determining that a sample from the mammal contains the absence of: (1) an increased level of a TMEM163 polypeptide or mRNA encoding the TMEM163 polypeptide, (2) an increased level of a HEY1 polypeptide or mRNA encoding the HEY1 polypeptide, (3) an increased level of an EYA4 polypeptide or mRNA encoding the EYA4 polypeptide, (4) an increased level of a TMEM98 polypeptide or mRNA encoding the TMEM98 polypeptide, (5) an increased level of a RLBP1 polypeptide or mRNA encoding the RLBP1 polypeptide, (6) an increased level of an ELOVL2 polypeptide or mRNA encoding the ELOVL2 polypeptide, (7) an increased level of a SERPINE2 polypeptide or mRNA encoding the SERPINE2 polypeptide, (8) an increased level of an ITGB3 polypeptide or mRNA encoding the ITGB3 polypeptide, (9) an increased level of an IGF1R polypeptide or mRNA encoding the IGF1R polypeptide, (10) a decreased level of a CD200R1 polypeptide or mRNA encoding the CD200R1 polypeptide, (11) a decreased level of a SIRPG polypeptide or mRNA encoding the SIRPG polypeptide, (12) a decreased level of a BTC polypeptide or mRNA encoding the BTC polypeptide, (13) a decreased level of an OR52N4 polypeptide or mRNA encoding the OR52N4 polypeptide, (14) a decreased level of an ETV7 polypeptide or mRNA encoding the ETV7 polypeptide, (15) a decreased level of a TOX polypeptide or mRNA encoding the TOX polypeptide, (16) a decreased level of an ARSG polypeptide or mRNA encoding the ARSG polypeptide, (17) a decreased level of an ITGB7 polypeptide or mRNA encoding the ITGB7 polypeptide, (18) a decreased level of a CDKN2A polypeptide or mRNA encoding the CDKN2A polypeptide, and (19) a decreased level of a DCD polypeptide or mRNA encoding the DCD polypeptide, wherein the sample is a tissue sample comprising a melanoma cell; and
(b) selecting the mammal for surgery to remove the PCM in the absence of an adjuvant therapy.

38. The method of claim 37, the method further comprising determining that the PCM has a Breslow depth of from about 0.1 mm to about 0.8 mm.

39. The method of claim 37, the method further comprising determining that the sample contains the absence of a mutation in a BRAF polypeptide and a mutation in a NRAS polypeptide.

40. The method of claim 36, wherein the mutation in the BRAF polypeptide is an amino acid substitution at residue 600 of the BRAF polypeptide.

41. The method of claim 40, wherein the mutation in the BRAF polypeptide is selected from the group consisting of a BRAFV600E substitution, a BRAFV600K substitution, a BRAFV600R substitution, and a BRAFV600D substitution.

42. The method of claim 36, wherein the mutation in the NRAS polypeptide is an amino acid substitution at residue 61 of the NRAS polypeptide.

43. The method of claim 42, wherein the mutation in the NRAS polypeptide is selected from the group consisting of a NRASQ61K substitution, a NRASQ61R substitution, a NRASQ61L substitution, a NRASQ61V substitution, and a NRASQ61H substitution.

44. The method of claim 34, wherein the mammal is a human.

45. The method of claim 34, wherein the adjuvant therapy comprises radiation therapy.

46. The method of claim 34, wherein the adjuvant therapy comprises administering an anti-cancer drug to the mammal.

47. The method of claim 46, wherein the anti-cancer drug is selected from the group consisting of nivolumab, ipilimumab, pembrolizumab, talimogene laherparepvec, vemurafenib, dabrafenib, encorafenib, trametinib, cobimetinib, binimetinib, atezolizumab, avelumab, and durvalumab.

48. (canceled)

49. A method for treating a mammal having a primary cutaneous melanoma (PCM), wherein the method comprises subjecting the mammal to a surgery to remove the PCM and administering an adjuvant therapy to the mammal, wherein the mammal is identified as having at least 11 of: (1) an increased level of a TMEM163 polypeptide or mRNA encoding the TMEM163 polypeptide, (2) an increased level of a HEY1 polypeptide or mRNA encoding the HEY1 polypeptide, (3) an increased level of an EYA4 polypeptide or mRNA encoding the EYA4 polypeptide, (4) an increased level of a TMEM98 polypeptide or mRNA encoding the TMEM98 polypeptide, (5) an increased level of a RLBP1 polypeptide or mRNA encoding the RLBP1 polypeptide, (6) an increased level of an ELOVL2 polypeptide or mRNA encoding the ELOVL2 polypeptide, (7) an increased level of a SERPINE2 polypeptide or mRNA encoding the SERPINE2 polypeptide, (8) an increased level of an ITGB3 polypeptide or mRNA encoding the ITGB3 polypeptide, (9) an increased level of an IGF1R polypeptide or mRNA encoding the IGF1R polypeptide, (10) a decreased level of a CD200R1 polypeptide or mRNA encoding the CD200R1 polypeptide, (11) a decreased level of a SIRPG polypeptide or mRNA encoding the CD200R1 polypeptide, (12) a decreased level of a BTC polypeptide or mRNA encoding the BTC polypeptide, (13) a decreased level of an OR52N4 polypeptide or mRNA encoding the OR52N4 polypeptide, (14) a decreased level of an ETV7 polypeptide or mRNA encoding the ETV7 polypeptide, (15) a decreased level of a TOX polypeptide or mRNA encoding the TOX polypeptide, (16) a decreased level of an ARSG polypeptide or mRNA encoding the ARSG polypeptide, (17) a decreased level of an ITGB7 polypeptide or mRNA encoding the ITGB7 polypeptide, (18) a decreased level of a CDKN2A polypeptide or mRNA encoding the CDKN2A polypeptide, and (19) a decreased level of a DCD polypeptide or mRNA encoding the DCD polypeptide, wherein a sample is a tissue sample comprising a melanoma cell.

50.-60. (canceled)

61. A method for treating a mammal having a PCM, wherein the method comprises subjecting the mammal to a surgery to remove the PCM in the absence of an adjuvant therapy, wherein the mammal is identified as lacking: (1) an increased level of a TMEM163 polypeptide or mRNA encoding the TMEM163 polypeptide, (2) an increased level of a HEY1 polypeptide or mRNA encoding the HEY1 polypeptide, (3) an increased level of an EYA4 polypeptide or mRNA encoding the EYA4 polypeptide, (4) an increased level of a TMEM98 polypeptide or mRNA encoding the TMEM98 polypeptide, (5) an increased level of a RLBP1 polypeptide or mRNA encoding the RLBP1 polypeptide, (6) an increased level of an ELOVL2 polypeptide or mRNA encoding the ELOVL2 polypeptide, (7) an increased level of a SERPINE2 polypeptide or mRNA encoding the SERPINE2 polypeptide, (8) an increased level of an ITGB3 polypeptide or mRNA encoding the ITGB3 polypeptide, (9) an increased level of an IGF1R polypeptide or mRNA encoding the IGF1R polypeptide, (10) a decreased level of a CD200R1 polypeptide or mRNA encoding the CD200R1 polypeptide, (11) a decreased level of a SIRPG polypeptide or mRNA encoding the SIRPG polypeptide, (12) a decreased level of a BTC polypeptide or mRNA encoding the BTC polypeptide, (13) a decreased level of an OR52N4 polypeptide or mRNA encoding the OR52N4 polypeptide, (14) a decreased level of an ETV7 polypeptide or mRNA encoding the ETV7 polypeptide, (15) a decreased level of a TOX polypeptide or mRNA encoding the TOX polypeptide, (16) a decreased level of an ARSG polypeptide or mRNA encoding the ARSG polypeptide, (17) a decreased level of an ITGB7 polypeptide or mRNA encoding the ITGB7 polypeptide, (18) a decreased level of a CDKN2A polypeptide or mRNA encoding the CDKN2A polypeptide, and (19) a decreased level of a DCD polypeptide or mRNA encoding the DCD polypeptide, wherein a sample is a tissue sample comprising a melanoma cell.

62.-71. (canceled)
